(19) United States
(12) Patent Application Publication
(10) Pub. No.: US 2007/0032643 A1
(43) Pub. Date: Feb. 8, 2007

(54) Cis-element regulating transcription, transcriptional regulatory factor binding specifically thereto and use of the same

(76) Inventors: Yoshihiko Nishio, Otsu-shi (JP); Hiroshi Maegawa, Kusatsu-shi (JP); Atsunori Kashiwagi, Otsu-shi (JP)

Correspondence Address: EDWARDS & ANGELL, LLP, P.O. BOX 55874, BOSTON, MA, 02205, US

(21) Appl. No.: 10/546,096

(22) PCT Filed: Feb. 19, 2004
(86) PCT No.: PCT/JP04/01924

§ 371 Date: Sep. 18, 2006

(30) Foreign Application Priority Data
Feb. 20, 2003 (JP) 2003-043398
Jun. 5, 2003 (JP) 2003-160856

(51) Int. Cl. G01N 33/567; C07H 21/04; C12P 21/06; C07K 14/705; C07K 16/28

(52) U.S. Cl. 536/23.5; 435/69.1; 435/320.1; 435/325; 530/350; 530/388.22; 435/7.2

(57) ABSTRACT

The present invention provides a novel fructose responsive transcription control cis-element and a transcriptional regulatory factor that interacts therewith, a non-human animal having them transferred or inactivated, a diagnostic method for genetic susceptibility to a metabolic disorder using them, and a screening method for a prophylactic or therapeutic drug for a metabolic disorder using them.

### FIELD OF THE INVENTION

The present invention relates to a novel transcription control cis-element in an SREBP-1c promoter, which is associated with genetic susceptibility to a metabolic disorder, a novel transcriptional regulatory factor capable of binding thereto, a screening method for a prophylactic or therapeutic substance for a metabolic disorder using them, a mutant promoter sequence of low sensitivity to a metabolic disorder, and a non-human transformant animal having the gene for the SREBP-1c promoter or the above-described transcriptional regulatory factor modified.

### BACKGROUND ART

The morbidity rate of type 2 diabetes mellitus has been rising over the past several decades. One important background factor of this trend is that a population in a state called metabolic syndrome, which comprises some metabolic abnormalities such as hyperlipemia, visceral obesity, abnormality of glucose tolerance, and hyperinsulinemia, has recently been increasing. Metabolic syndrome is inducible by environmental factors such as high-calorie diets and a lack of exercise in people with an unidentified genetic background.

Since high-fructose diets induce metabolic disruptions similar to metabolic syndrome in rats, rats loaded with a high-fructose diet are used as an established animal model of metabolic syndrome. Nagai et al. (non-patent document 1) discloses that in rats loaded with a high-fructose diet, the expression of sterol regulatory element binding protein-1 (SREBP-1), a key transcription factor for the expression of a class of lipid synthases in the liver, is induced, whereas the expression of the peroxisome proliferator activated receptor α (PPARα), a ligand-responding nuclear receptor that regulates the expression of a class of enzymes involved in fatty acid oxidation, is downregulated. These changes in the expression of transcription factors may play a central role in the onset of metabolic disruptions in rats loaded with a high-fructose diet.

Furthermore, Nagata et al. (non-patent document 2) discloses that strain-related differences exist in obesity and lipid metabolic abnormalities due to a high-fructose diet in mice, and that these differences are involved in differences in the expression of SREBP-1c in the liver. Specifically, CBA mice had increases in body weight and serum lipid due to a high-fructose diet, whereas DBA/2 mice had no changes; these metabolic abnormalities correlated positively with the expression amount of hepatic SREBP-1c mRNA.

However, the mechanism behind the induction of the expression of the SREBP-1 gene by fructose has not yet been elucidated, nor has been identified a genetic disposition for metabolic abnormalities due to a high-fructose diet.

Non-patent document 1: Nagai et al., “American Journal of Physiology, Endocrinology and Metabolism”, (USA), 2002, Vol. 282, No. 5, p. E1180-1190

Non-patent document 2: Nagata et al., “Tonyobyo (Diabetes Mellitus)”, Japan Diabetes Society, Apr. 15, 2002, Vol. 45, Supplementary No. 2, p. S247

### SUMMARY OF THE INVENTION

An object of the present invention is to elucidate the mechanism of induction of the expression of the SREBP-1c gene by fructose in mammals and identify a factor that determines genetic susceptibility to a diet-related metabolic disorder, so as to provide an effective prophylactic or therapeutic agent for a metabolic disorder.

The present inventors conducted diligent investigations aiming at accomplishing the above object, and found that there is a difference in one base in the promoter region of the SREBP-1c gene between CBA mice, which exhibit a lipid metabolic abnormality due to a high-fructose diet, and DBA mice, which exhibit almost no such metabolic abnormality. Furthermore, the inventors conducted a gel shift assay using nucleic acid probes having base sequences comprising the mutated sites in these two groups of mice, and demonstrated the presence of two transcriptional regulatory factors capable of specifically binding only to the base sequence derived from the former strain of mice. As a result of a determination of the amino acid sequences of these transcriptional regulatory factors by TOF-MS analysis, the factors were found to be proteins similar to publicly known Nonamer Binding Protein (NBP) and RNA binding motif protein, X chromosome retrogene (RBMX). The expression of these transcriptional regulatory factors increased remarkably after meals, especially after ingestion of a high-fructose diet, but there was almost no expression during fasting; this expression correlated well with the expression of the SREBP-1c gene.

The present inventors also analyzed publicly known promoters of the human SREBP-1c gene, and confirmed the presence of a sequence homologous to the above-described base sequence comprising the mutated site in CBA mice, and found that a polymorphism similar to that in mice may occur in humans.

The present inventors conducted further investigations based on these findings, and developed the present invention.

Accordingly, the present invention provides:
[1] a nucleic acid consisting of the same or substantially the same base sequence as a portion of the base sequence shown by SEQ ID NO:1, and comprising the guanine shown by base number 112 in the base sequence,
[2] a nucleic acid characterized by (1) and (2) below:
(1) comprises a base sequence having one or more bases substituted, deleted, inserted or added in the same or substantially the same base sequence as a portion of the base sequence shown by SEQ ID NO:1, and comprising the base sequence or the guanine shown by base number 112
(2) a transcriptional regulatory factor capable of binding to a base sequence consisting of the guanine shown by base number 112 and a base adjoining thereto in the base sequence shown by SEQ ID NO:1 cannot bind to the nucleic acid
[3]the nucleic acid described in [2] above, wherein the guanine shown by base number 112 in the base sequence shown by SEQ ID NO:1 is substituted by another base,
[4] the nucleic acid described in [3] above, wherein the another base is adenine,
[5] a diagnostic method for genetic susceptibility to a metabolic disorder in a test animal, which comprises detecting a portion of the base sequence shown by SEQ ID NO:1 comprising the guanine shown by base number 112 in the base sequence, or a corresponding base sequence, in the SREBP-1c promoter,
[6] the method described in [5] above, wherein the metabolic disorder is a sugar or lipid metabolic disorder,
[7] a screening method for a prophylactic or therapeutic substance for a metabolic disorder, which comprises using both (a) below and (b) and/or (c) below,
(a) a DNA having the same or substantially the same base sequence as a portion of the base sequence shown by SEQ ID NO:1, comprising the base sequence or the guanine shown by base number 112,
(b) a protein comprising the same or substantially the same amino acid sequence as the amino acid sequence shown by SEQ ID NO:3 or a partial peptide thereof or a salt thereof
(c) a protein comprising the same or substantially the same amino acid sequence as the amino acid sequence shown by SEQ ID NO:5 or a partial peptide thereof or a salt thereof
[8] the method described in [7] above, wherein the metabolic disorder is a sugar or lipid metabolic disorder,
[9] the method described in [7] above, which comprises detecting the inhibition of the binding of (a) above and (b) and/or (c) above in the presence of a test substance,
[10] the method described in [7] above, which comprises loading a sugar on an animal cell having a gene under the control of a promoter comprising (a) above, and comparing the expression of the gene between in the presence and in the absence of a test substance,
[11] the method described in [10] above, wherein the animal cell is capable of producing (b) and/or (c) above,
[12] the method described in [10] above, wherein the animal cell is a hepatocyte,
[13] the method described in [10] above, wherein the sugar is fructose,
[14] the method described in [10] above, which comprises loading a sugar on an animal having a gene under the control of a promoter comprising a DNA having the same or substantially the same base sequence as a portion of the base sequence shown by SEQ ID NO:1, and comprising the guanine shown by base number 112 in the base sequence, and comparing the expression of the gene in the liver between with and without administration of a test substance,
[15] the method described in [14] above, wherein the sugar is fructose,
[16] a prophylactic or therapeutic agent for a metabolic disorder, which contains a substance that suppresses the production or activity of (a) and/or (b) below,
(a) a protein comprising the same or substantially the same amino acid sequence as the amino acid sequence shown by SEQ ID NO:3 or a partial peptide thereof or a salt thereof
(b) a protein having the same or substantially the same amino acid sequence as the amino acid sequence shown by SEQ ID NO:5 or a partial peptide thereof or a salt thereof
[17] the agent described in [16] above, wherein the metabolic disorder is a sugar or lipid metabolic disorder,
[18] the agent described in [16] above, wherein the substance that suppresses the activity is an antibody against (a) above and/or an antibody against (b) above,
[19] the agent described in [16] above, wherein the substance that suppresses the activity is a DNA having the same or substantially the same base sequence as a portion of the base sequence shown by SEQ ID NO:1, and comprising the guanine shown by base number 112 in the base sequence,
[20] the agent described in [16] above, wherein the substance that suppresses the production is (c) and/or (d) below,
(c) a nucleic acid comprising a base sequence complementary to the base sequence that encodes (a) above, or a portion thereof
(d) a nucleic acid comprising a base sequence complementary to the base sequence that encodes (b) above, or a portion thereof
[21] a prophylactic or therapeutic method for a metabolic disorder, which comprises administering an effective amount of a substance that suppresses the production or activity of (a) and/or (b) below to a mammal,
(a) a protein having the same or substantially the same amino acid sequence as the amino acid sequence shown by SEQ ID NO:3 or a partial peptide thereof or a salt thereof
(b) a protein having the same or substantially the same amino acid sequence as the amino acid sequence shown by SEQ ID NO:5 or a partial peptide thereof or a salt thereof
[22] use of a substance that suppresses the production or activity of (a) and/or (b) below, for the production of a prophylactic or therapeutic agent for a metabolic disorder,
(a) a protein comprising the same or substantially the same amino acid sequence as the amino acid sequence shown by SEQ ID NO:3 or a partial peptide thereof or a salt thereof
(b) a protein comprising the same or substantially the same amino acid sequence as the amino acid sequence shown by SEQ ID NO:5 or a partial peptide thereof or a salt thereof
[23] a protein or peptide characterized by (1) and (2) below or a salt thereof:
(1) comprises an amino acid sequence having one or more amino acids substituted, deleted, inserted, or added, in a protein comprising the same or substantially the same amino acid sequence as the amino acid sequence shown by SEQ ID NO:3 or a partial peptide thereof or a salt thereof,
(2) binds to the same or substantially the same base sequence as a portion of the base sequence shown by SEQ ID NO:1, comprising the guanine shown by base number 112 in the base sequence, but does not activate promoters comprising the base sequence
[24] a protein or peptide characterized by (1) and (2) below or a salt thereof:
(1) comprises an amino acid sequence having one or more amino acids substituted, deleted, inserted, or added, in a protein comprising the same or substantially the same amino acid sequence as the amino acid sequence shown by SEQ ID NO:5 or a partial peptide thereof or a salt thereof,
(2) binds to the same or substantially the same base sequence as a portion of the base sequence shown by SEQ ID NO:1, comprising the guanine shown by base number 112 in the base sequence, but does not activate promoters comprising the base sequence
[25] a prophylactic or therapeutic agent for a metabolic disorder, which contains the protein or peptide described in [23] above or a salt thereof, and/or the protein or peptide described in [24] above or a salt thereof,
[26] a diagnostic reagent for a metabolic disorder, which contains (a) and/or (b) below:
(a) an antibody against a protein comprising the same or substantially the same amino acid sequence as the amino acid sequence shown by SEQ ID NO:3 or a partial peptide thereof or a salt thereof
(b) an antibody against a protein comprising the same or substantially the same amino acid sequence as the amino acid sequence shown by SEQ ID NO:5 or a partial peptide thereof or a salt thereof
[27] a diagnostic reagent for a metabolic disorder, which contains (a) and/or (b) below:
(a) a nucleic acid comprising the base sequence that encodes a protein having the same or substantially the same amino acid sequence as the amino acid sequence shown by SEQ ID NO:3 or a portion thereof
(b) a nucleic acid comprising the base sequence that encodes a protein having the same or substantially the same amino acid sequence as the amino acid sequence shown by SEQ ID NO:5 or a portion thereof
[28] a non-human transgenic animal incorporating a gene under the control of a promoter comprising a DNA having the same or substantially the same base sequence as a portion of the base sequence shown by SEQ ID NO:1, and comprising the guanine shown by base number 112 in the base sequence
[29] the non-human transgenic animal described in [28] above, wherein an endogenous SREBP-1c gene characterized by (1) below:
(1) comprises a promoter to which (a) and/or (b) below cannot bind:
(a) a protein comprising the same or substantially the same amino acid sequence as the amino acid sequence shown by SEQ ID NO:3 or a partial peptide thereof or a salt thereof
(b) a protein comprising the same or substantially the same amino acid sequence as the amino acid sequence shown by SEQ ID NO:5 or a partial peptide thereof or a salt thereof is substituted by an SREBP-1c gene characterized by (2) below:
(2) is under the control of a promoter comprising a DNA having the same or substantially the same base sequence as a portion of the base sequence shown by SEQ ID NO:1, and comprising the guanine shown by base number 112 in the base sequence,
[30] a non-human transgenic animal incorporating a gene under the control of a promoter characterized by (1) and (2) below:
(1) comprises a DNA having one or more bases substituted, deleted, inserted, or added, in the same or substantially the same base sequence as a portion of the base sequence shown by SEQ ID NO:1, and comprising the guanine shown by base number 112 in the base sequence,
(2) (a) and/or (b) below cannot bind:
(a) a protein comprising the same or substantially the same amino acid sequence as the amino acid sequence shown by SEQ ID NO:3 or a partial peptide thereof or a salt thereof
(b) a protein comprising the same or substantially the same amino acid sequence as the amino acid sequence shown by SEQ ID NO:5 or a partial peptide thereof or a salt thereof
[31] the non-human transgenic animal described in [30] above, wherein the endogenous SREBP-1c gene comprising a promoter having the same or substantially the same base sequence as the base sequence shown by SEQ ID NO:1 is substituted by an SREBP-1c gene under the control of a promoter characterized by (1) and (2) above,
[32] a non-human transgenic animal incorporating (a) and/or (b) below:
(a) a DNA that encodes a protein comprising the same or substantially the same amino acid sequence as the amino acid sequence shown by SEQ ID NO:3 or a partial peptide thereof
(b) a DNA that encodes a protein comprising the same or substantially the same amino acid sequence as the amino acid sequence shown by SEQ ID NO:5 or a partial peptide thereof and
[33] a non-human animal having (a) and/or (b) below inactivated:
(a) a DNA that encodes a protein comprising the same or substantially the same amino acid sequence as the amino acid sequence shown by SEQ ID NO:3
(b) a DNA that encodes a protein comprising the same or substantially the same amino acid sequence as the amino acid sequence shown by SEQ ID NO:5.

Because the novel transcription control cis-element in the SREBP-1c promoter, which was discovered in the present invention, promotes the expression of SREBP-1c by interacting with a transcriptional regulatory factor that specifically binds thereto, a nucleic acid having a base sequence comprising the cis-element can be used in combination with the transcriptional regulatory factor to screen for a compound that regulates the expression of the SREBP-1c gene, hence for a candidate compound for a prophylactic or therapeutic drug for a metabolic disorder. Furthermore, because the nucleic acid is capable of detecting the presence or absence of a mutation in the cis-element in the SREBP-1c promoter, it can be used to diagnose genetic susceptibility to a diet-related metabolic disorder in a mammal.

### BRIEF DESCRIPTION OF THE DRAWINGS

FIG. 1 shows serum TG concentrations (FIG. 1A), SREBP-1c mRNA levels (FIG. 1B), PPARα mRNA levels (FIG. 1C) and FAS mRNA levels (FIG. 1D) in various feeding groups of DBA/2 JN Crj mice (I) and CBA/JN Crj mice (II). In FIG. 1A, FIG. 1B, FIG. 1C and FIG. 1D, the ordinate indicates serum TG concentration (mg/dl), SREBP-1c mRNA level (relative value based on CF group as 1 unit), PPARα mRNA level (relative value based on CF group as 1 unit), and FAS mRNA level (relative value based on CF group as 1 unit), respectively, and the bar graphs show the results from the CF group, the CR group, the FF group, and the FR group, respectively, from left. In these figures, * indicates p<0.01, and ** indicates p<0.05.

FIG. 2 shows SREBP-1c mRNA levels (FIG. 2A) and FAS mRNA levels (FIG. 2B) in various stimulation groups of primary hepatocytes derived from DBA/2 JN Crj mice (I) and CBA/JN Crj mice (II). In FIG. 2A and FIG. 2B, the ordinate indicates SREBP-1c MRNA level (relative value based on glucose stimulation group as 1 unit) and FAS mRNA level (relative value based on glucose stimulation group as 1 unit), respectively, and the bar graphs show the results from the glucose stimulation group, the fructose stimulation group, and the glucose+insulin stimulation group, respectively, from left. In these figures, * indicates p<0.01, and ** indicates p<0.05.

FIG. 3 shows the results of an electrophoretic mobility shift assay (EMSA) for the presence of a transcriptional regulatory factor that binds to the fructose responsive element of the present invention and the regulation of the expression thereof. FIG. 3A shows the results of an EMSA of a nuclear extract derived from CBA/JN Crj mice in the FR group with the CBA probe or the DBA probe (lanes 1 and 2: CBA probe, lanes 3 and 4: DBA probe; lanes 1 and 3: in the presence of cold probe, lanes 2 and 4: in the absence of cold probe). FIG. 3B shows the results of an EMSA of a nuclear extract derived from CBA/JN Crj mice or DBA/2 JN Crj mice in the FR group with the CBA probe (lane 1: nuclear extract derived from CBA/JN Crj mouse; lane 2: nuclear extract derived from DBA/2 JN Crj mice). FIG. 3C shows the results of an EMSA of a nuclear extract derived from CBA/JN Crj mice in the CF group or the FR group with the CBA probe (lanes 1 and 2: CF group, lanes 3 and 4: FR group; lanes 1 and 4: in the absence of cold probe, lanes 2 and 3: in the presence of cold probe).

FIG. 4 shows a comparison of the base sequences of the mouse (FIG. 4A) and human (FIG. 4B) SREBP-1c promoters [candidate binding site: candidate binding site of the transcriptional regulatory factor of the present invention (fructose responsive element); SNP (G->A): indicates the occurrence of G→A polymorphism (SNP) in the bold-faced “G”; LXRE: Liver X Receptor (LXR) responsive element; NF-Y: NF-Y binding site; E-box: E-box; SRE: sterol responsive element; SP1: SP1 binding site; homology site: site homologous to the above-described candidate binding site].

### BEST MODE FOR EMBODIMENT OF THE INVENTION

The present invention provides a novel transcription control cis-element capable of promoting the transcription of a gene downstream thereof in response to food ingestion (sugar loading), especially to a high-fructose diet (fructose loading) (hereinafter also referred to as “fructose responsive element (FRE)” and a nucleic acid comprising the cis-element.

The fructose responsive element (FRE) of the present invention has the same or substantially the same base sequence as a partial base sequence comprising the guanine shown by base number 112 (hereinafter also abbreviated as “G₁₁₂”) in the base sequence of an SREBP-1c promoter derived from a mouse of the CBA, C3H or other strain, shown by SEQ ID NO:1, preferably a partial base sequence consisting of about 5 to about 30 bases, more specifically a partial base sequence of a total length of about 5 to about 30 bases consisting of G₁₁₂, 0 to 20 bases upstream of the 5′ end thereof, and 0 to 20 bases downstream of the 3′ end of G₁₁₂.

“Substantially the same base sequence” refers to the above-described partial base sequence which is 1) a base sequence having one or more bases (preferably 1 to several bases) other than G₁₁₂ substituted by another base, 2) a base sequence having one or more bases (preferably 1 to several bases) deleted, 3) a base sequence having one or more bases (preferably 1 to several bases) inserted, or a base sequence comprising a combination thereof, and which is capable of promoting the transcription of a gene downstream thereof in response to food ingestion (sugar loading), especially to a high-fructose diet (fructose loading). Transcription promoting activity can be determined by the binding assay with the transcriptional regulatory factor described below, or by detecting an increase in the expression of a reporter gene (e.g., luciferase, Green Fluorescent Protein (GFP) and the like) under the control of a promoter comprising the base sequence to be examined due to sugar (e.g., fructose) loading. As preferable examples of “substantially the same base sequence”, partial base sequences comprising a base corresponding to G₁₁₂ in an SREBP-1c gene promoter derived from a mammal other than mice (e.g., human, rat, rabbit, guinea pig, hamster, bovine, horse, sheep, monkey, dog, cat and the like) of a strain or individual showing a tendency for a metabolic abnormality such as increased serum lipid after meals (especially after ingestion of a high-fructose diet), and the like can be mentioned. Specifically, for example, a partial base sequence comprising the guanine shown by base number 39, preferably a partial base sequence consisting of about 5 to about 30 bases, more specifically a partial base sequence of a total length of about 5 to about 30 bases consisting of the guanine, 0 to 20 bases upstream of the 5′ end thereof, and 0 to 20 bases downstream of the 3′ end of the same guanine, in the base sequence of the human-derived SREBP-1c promoter, shown by SEQ ID NO:13, can be mentioned.

The nucleic acid comprising the FRE of the present invention may be any nucleic acid having the above-described base sequence of the FRE of the present invention or a base sequence having one or more bases added upstream of the 5′ end and/or downstream of the 3′ end of FRE (but excluding nucleic acids comprising the entire base sequence shown by SEQ ID NO:1). The length of base sequence added is not subject to limitation; for example, base sequences comprising an SREBP-1c promoter sequence further upstream of the sequence upstream of the 5′ end of G₁₁₂ of the base sequence shown by SEQ ID NO:1 (e.g., base sequence shown by base numbers 1 to 637 in the base sequence shown by SEQ ID NO:6 and the like) are also included.

The nucleic acid may be a DNA, an RNA, or a DNA/RNA chimera, and can be selected as appropriate according to the intended use (e.g., expression promoter, diagnostic probe, therapeutic decoy nucleotide and the like), with preference given to a DNA. The nucleic acid may be single-stranded or double-stranded; in the case of a double-stranded nucleic acid, it may be a hybrid of a DNA strand and an RNA strand. Additionally, the nucleic acid may be physiologically acceptable salts with acid or base. For example, physiologically acceptable acid addition salts are preferred. As such salts, salts with inorganic acids (for example, hydrochloric acid, phosphoric acid, hydrobromic acid, sulfuric acid) or salts with organic acids (for example, acetic acid, formic acid, propionic acid, fumaric acid, maleic acid, succinic acid, tartaric acid, citric acid, malic acid, oxalic acid, benzoic acid, methanesulfonic acid, benzenesulfonic acid) and the like particularly used.

A nucleic acid (preferably DNA) comprising the FRE of the present invention can be prepared from a genomic DNA extracted from cells [e.g., hepatocyte, splenocyte, nerve cell, glial cell, pancreatic β cell, myelocyte, mesangial cell, Langerhans' cell, epidermal cell, epithelial cell, endothelial cell, fibroblast, fibrocyte, myocyte, adipocyte, immune cell (e.g., macrophage, T cell, B cell, natural killer cell, mast cell, neutrophil, basophil, eosinophil, monocyte), megakaryocyte, synovial cell, chondrocyte, bone cell, osteoblast, osteoclast, mammary gland cell or interstitial cell, or a corresponding precursor cell, stem cell or cancer cell thereof, and the like] derived from a human or another mammal (e.g., mouse, rat, rabbit, guinea pig, hamster, bovine, horse, sheep, monkey, dog, cat and the like), preferably from a strain or individual showing a tendency for a metabolic abnormality such as increased serum lipid after meals (especially after ingestion of high-fructose diet), particularly preferably from a CBA or C3H mouse, or any tissue where such cells are present [e.g., brain or any portion of brain (e.g., olfactory bulb, amygdaloid nucleus, basal ganglia, hippocampus, thalamus, hypothalamus, cerebral cortex, medulla oblongata, cerebellum), spinal cord, hypophysis, stomach, pancreas, kidney, liver, gohad, thyroid, gallbladder, bone marrow, adrenal gland, skin, muscle, lung, gastrointestinal tract (e.g., large intestine and small intestine), blood vessel, heart, thymus, spleen, salivary gland, peripheral blood, prostate, testicle, ovary, placenta, uterus, bone, cartilage, joint, skeletal muscle, and the like], by cloning a genomic DNA comprising the promoter region with a publicly known SREBP-1c gene promoter sequence (for example, described in Amemiya-Kudo et al., Journal of Biological Chemistry, 2000, Vol. 275, No. 40, p. 31078-31085; GenBank accession number: AB046200) as a probe, cleaving the DNA into a DNA fragment comprising the desired partial promoter sequence using a DNA degradation enzyme, for example, an appropriate restriction enzyme, separating the fragment by gel electrophoresis, thereafter recovering the desired band, and purifying the DNA. Alternatively, an SREBP-1c promoter partial sequence comprising the FRE of the present invention can be amplified and isolated by a PCR using a primer synthesized on the basis of a publicly known SREBP-1c gene promoter sequence with a crude extract of the above-described cell or a genomic DNA isolated therefrom as a template.

A nucleic acid comprising the FRE of the present invention can also be obtained by chemically synthesizing a nucleic acid having a partial base sequence of the base sequence shown by SEQ ID NO:1, comprising G₁₁₂, or substantially the same base sequence as the base sequence on the basis of the base sequence shown by SEQ ID NO:1 using a commercially available DNA/RNA synthesizer.

In the case of chemical synthesis, the nucleic acid may be a type of polynucleotide other than deoxyribonucleotide and ribonucleotide, that is an N-glycoside of the purine or pyrimidine base, or another polymer having a non-nucleotide backbone (for example, commercially available protein nucleic acids and synthetic sequence specific nucleic acid polymers) or another polymer having a special bond (however, this polymer comprises a nucleotide having a configuration that allows base pairing or base attachment as found in DNA and RNA) or the like. These may be those having a known modification added thereto, for example, those with a label known in the relevant field, those with a cap, those methylated, those having 1 or more naturally occurring nucleotides substituted by analogues, those modified with an intramolecular nucleotide, for example, those having a non-charge bond (for example, methylphosphonate, phospho triester, phosphoramidate, carbamate and the like), those having a charged bond or a sulfur containing bond (for example, phosphorothioate, phosphorodithioate and the like), for example, those having a side chain group of a protein (nuclease, nuclease inhibitor, toxin, antibody, signal peptide, poly-L-lysine and the like) or a sugar (for example, monosaccharide and the like), those having an intercalating compound (for example, acridine, psoralen and the like), those containing a chelate compound (for example, metals, radioactive metals, boron, oxidizing metals and the like), or those containing an alkylating agent, or those having a modified bond (for example, α-anomer type nucleic acid and the like). Here, “nucleotide” and “nucleic acid” may include not only those containing the purine and pyrimidine bases, but also those containing another modified heterocycle type base. These modified products may contain a methylated purine and pyrimidine, an acylated purine and pyrimidine, or another heterocycle. The modified nucleotide may also have their sugar portion modified by, for example, substitution of one or more hydroxyl groups by a halogen, an aliphatic group (e.g., C₁₋₆ alkyl group) and the like, or conversion to a functional group such as an ether or an amine. As specific examples of the modified nucleic acid, sulfur derivatives and thiophosphate derivatives of nucleic acids, and those resistant to the decomposition like polynucleosideamide or oligonucleosideamide can be mentioned, which, however, are not to be construed as limiting.

Such a modified nucleic acid is useful in increasing in vivo stability and improving cell permeation when used as, for example, a therapeutic decoy nucleotide.

When using a nucleic acid comprising the FRE of the present invention as a gene expression promoter, a base sequence to confer a basal promoter activity, such as the TATA box, is added downstream of the FRE. Furthermore, another transcription control cis-sequence (e.g., CAAT box, GC box and the like) can also be placed at an appropriate position.

The present invention also provides a mutant SREBP-1c promoter having a mutation in the above-described fructose responsive element of the present invention, to which a transcriptional regulatory factor capable of binding to the element (that is, the NBP and/or RBMX analogous protein described below) cannot bind, or a partial polynucleotide thereof comprising the mutated site. That is, the mutated FRE of the present invention or the mutant SREBP-1c promoter comprising the mutant FRE is a nucleic acid comprising a base sequence having one or more bases (preferably 1 to several bases) substituted, deleted, inserted, or added, in the same or substantially the same base sequence as the base sequence shown by SEQ ID NO:1 or a portion of the base sequence comprising the guanine shown by base number 112 (G₁₁₂), to which a transcriptional regulatory factor capable of binding to a base sequence consisting of G₁₁₂ and a base adjoining thereto in the base sequence shown by SEQ ID NO:1 cannot bind. Here, “substantially the same base sequence” has the same definition as above. The “adjoining base” may be located upstream of the 5′ end of G₁₁₂ or downstream of the 3′ end, or both. The base sequence consisting of G₁₁₂ and a base adjoining thereto is a base sequence of a total length of about 5 to about 30 bases, preferably consisting of about 5 to about 30 bases, more specifically of G₁₁₂, 0 to 20 bases upstream of the 5′ end thereof, and 0 to 20 bases downstream of the 3′ of G₁₁₂.

The mutant FRE and a mutant SREBP-1c promoter comprising the same can be used as probes for detecting the mutation in an SREBP-1c promoter of an animal individual, and the mutant SREBP-1c promoter is useful as a transgene and the like for the preparation of a transgenic animal model that is resistant to high-fructose diets.

Preferably, the mutated FRE of the present invention or a mutant SREBP-1c promoter comprising the same is the above-described nucleic acid having G₁₁₂ in the base sequence shown by SEQ ID NO:1 substituted by another base, and is particularly preferably the above-described nucleic acid having G₁₁₂ substituted by adenine.

The mutant FRE of the present invention or a mutant SREBP-1c promoter comprising the same can be prepared from a genomic DNA extracted from cells [e.g., hepatocyte, splenocyte, nerve cell, glial cell, pancreatic β cell, myelocyte, mesangial cell, Langerhans' cell, epidermal cell, epithelial cell, endothelial cell, fibroblast, fibrocyte, myocyte, adipocyte, immune cell (e.g., macrophage, T cell, B cell, natural killer cell, mast cell, neutrophil, basophil, eosinophil, monocyte), megakaryocyte, synovial cell, chondrocyte, bone cell, osteoblast, osteoclast, mammary gland cell or interstitial cell, or a corresponding precursor cell, stem cell or cancer cell thereof, and the like] derived from a human or another mammal (e.g., mouse, rat, rabbit, guinea pig, hamster, bovine, horse, sheep, monkey, dog, cat and the like) having the mutant promoter, for example, from a strain or individual that does not exhibit a metabolic abnormality such as increased serum lipid after meals (especially after ingestion of high-fructose diet), particularly preferably from a DBA or C57BL mouse, or any tissue where such cells are present [e.g., brain or any portion of brain (e.g., olfactory bulb, amygdaloid nucleus, basal ganglia, hippocampus, thalamus, hypothalamus, cerebral cortex, medulla oblongata, cerebellum), spinal cord, hypophysis, stomach, pancreas, kidney, liver, gonad, thyroid, gallbladder, bone marrow, adrenal gland, skin, muscle, lung, gastrointestinal tract (e.g., large intestine and small intestine), blood vessel, heart, thymus, spleen, salivary gland, peripheral blood, prostate, testicle, ovary, placenta, uterus, bone, cartilage, joint, skeletal muscle, and the like], by cloning a genomic DNA comprising the promoter region with a publicly known SREBP-1c gene promoter sequence (for example, described in Amemiya-Kudo et al., Journal of Biological Chemistry, 2000, Vol. 275, No. 40, p. 31078-31085; GenBank accession number: AB046200) as a probe, cleaving the DNA into a DNA fragment comprising the desired (partial) promoter sequence using a DNA degradation enzyme, for example, an appropriate restriction enzyme, separating the fragment by gel electrophoresis, thereafter recovering the desired band, and purifying the DNA. Alternatively, the mutant FRE of the present invention or a mutant SREBP-1c promoter comprising the same can be isolated by a PCR using a primer synthesized on the basis of a publicly known SREBP-1c gene promoter sequence with a crude extract of the above-described cell or a genomic DNA isolated therefrom as a template.

The mutated FRE of the present invention or a mutant SREBP-1c promoter comprising the same can also be obtained by site-directed mutagenesis by a PCR using a publicly known SREBP-1c gene promoter as a template, with an oligonucleotide having a base sequence having one or more bases (preferably 1 to several bases) substituted, deleted, inserted, or added in the FRE base sequence of the present invention as one primer. Whether the thus-obtained mutant FRE or a mutant SREBP-1c promoter comprising the same does not promote the transcription of a gene downstream thereof in response to food ingestion (sugar loading), especially to a high-fructose diet (fructose loading), can be determined by the binding assay with a transcriptional regulatory factor described below, or by examining changes in the expression of a reporter gene (e.g., luciferase, Green Fluorescent Protein (GFP) and the like) under the control of the mutant promoter due to sugar (e.g., fructose) loading.

Alternatively, the mutated FRE of the present invention or a mutant SREBP-1c promoter comprising the same can also be obtained by chemically synthesizing a base sequence having one or more bases (preferably 1 to several bases) substituted, deleted, inserted, or added in the FRE base sequence in a publicly known SREBP-1c promoter, using a commercially available DNA/RNA autosynthesizer in the same manner as above.

The present invention also provides a diagnostic method for genetic susceptibility to a metabolic disorder in a test animal (for example, human or another mammal) by detecting a mutation in the fructose responsive element in an SREBP-1c promoter. That is, the method comprises detecting a portion of the base sequence comprising G₁₁₂ in the base sequence shown by SEQ ID NO:1 (that is, FRE derived from CBA mouse and the like) or a base sequence corresponding thereto (that is, another FRE of the present invention or mutated FRE of the present invention).

The above-described metabolic disorder include, for example, metabolic disorder due to diet (especially high fructose diet), for example, glucose or lipid metabolic disorder (e.g., hypertriglyceridemia, hyper-LDL-cholesteremia, hypo-HDL-cholesterolemia, obesity, abnormality of glucose tolerance, fasting blood glucose disorder, hyperinsulinemia, hypertension, albuminuria, and the like) and the like.

As a method of detecting a mutation in FRE, any publicly known method of SNP detection can be used. As examples of the detection method, a method wherein hybridization is conducted with accurate control of stringency in accordance with, for example, the method of Wallace et al. (Proc. Natl. Acad. Sci. USA, 80, 278-282 (1983)), using a genomic DNA extracted from cells of a test animal as a sample, with the above-described FRE of the present invention or a nucleic acid comprising the same, or the mutated FRE of the present invention or a nucleic acid comprising the same, as a probe, to detect only a sequence that is completely complementary to the probe, a method wherein hybridization is conducted with gradual reductions in reaction temperature from denaturation temperature using mixed probes prepared by labeling one of the FRE of the present invention or a nucleic acid comprising the same and the mutated FRE of the present invention or a nucleic acid comprising the same, and leaving the other non-labeled, to allow a sequence that is completely complementary to one probe to be hybridized in advance to thereby prevent its cross-reaction with a probe with a mismatch, and the like can be mentioned.

Detection of a mutation in FRE can also be performed by a publicly known PCR-based method of SNP detection, for example, PCR-SSCP method, allele-specific PCR, PCR-SSOP method, DGGE method, RNase protection method, PCR-RFLP method and the like. In the case of the PCR-SSCP method, for example, a PCR is conducted using an SREBP-1c promoter partial sequence upstream of the 5′ end of the FRE of the present invention as a sense primer and an SREBP-1c promoter complementary strand partial sequence downstream of the 3′ end of FRE as an antisense primer, with a test animal cell extract or a genomic DNA purified therefrom as a template (one of the primers and substrate nucleotide labeled previously), the resulting amplified fragments are rendered single-stranded and then subjected to non-denatured gel electrophoresis, and primary structural polymorphism can be detected on the basis of the difference in their mobility.

The present invention also provides two kinds of transcriptional regulatory factor capable of binding to the FRE of the present invention (hereinafter also referred to as “the transcriptional regulatory factor of the present invention”). The transcriptional regulatory factor possesses an activity to specifically bind to the FRE of the present invention to promote the transcription of a gene located downstream thereof, and possesses a DNA binding characteristic of the inability to bind to the mutated FRE of the present invention.

Specifically, the transcriptional regulatory factor of the present invention is (1) a protein comprising the same or substantially the same amino acid sequence as the amino acid sequence shown by SEQ ID NO:3, or (2) a protein comprising the same or substantially the same amino acid sequence as the amino acid sequence shown by SEQ ID NO:5. The protein comprising the same or substantially the same amino acid sequence as the amino acid sequence shown by SEQ ID NO:3 is a publicly known protein called Nonamer Binding Protein (NBP), identified as a protein that specifically binds to a conserved 9-mer sequence present in the vicinity of the recombination site in a rearrangement of the immunoglobulin or T cell receptor gene (Gene Dev., 3: 1801-1813, 1989). On the other hand, the protein comprising the same or substantially the same amino acid sequence as the amino acid sequence shown by SEQ ID NO:5 is one of the proteins belonging to the nuclear RNA binding protein family, which have an RNA binding motif, and is encoded by a gene similar to the RNA binding motif protein, X chromosome retrogene (RBMX) gene, which is known to be present on the X chromosome in humans and mice (Nature Genet., 22: 223-224, 1999). Hereinafter, the former is also referred to as “the NBP of the present invention”, and the latter as the “RBMX analogous protein of the present invention”.

The transcriptional regulatory factor of the present invention may be a protein derived from a cell (e.g., hepatocyte, splenocyte, nerve cell, glial cell, pancreatic β cell, myelocyte, mesangial cell, Langerhans' cell, epidermal cell, epithelial cell, goblet cell, endothelial cell, smooth muscle cell, fibroblast, fibrocyte, myocyte, adipocyte, immune cell (e.g., macrophage, T cell, B cell, natural killer cell, mast cell, neutrophil, basophil, eosinophil, monocyte), megakaryocyte, synovial cell, chondrocyte, bone cell, osteoblast, osteoclast, mammary gland cell, hepatocyte or interstitial cell, or a corresponding precursor cell, stem cell or cancer cell thereof, and the like) of a mammal (for example, human, mouse, rat, rabbit, sheep, swine, bovine, horse, cat, dog, monkey, chimpanzee and the like), or any tissue where such cells are present, for example, brain or any portion of brain (e.g., olfactory bulb, amygdaloid nucleus, basal ganglia, hippocampus, thalamus, hypothalamus, cerebral cortex, medulla oblongata, cerebellum), spinal cord, hypophysis, stomach, pancreas, kidney, liver, gonad, thyroid, gallbladder, bone marrow, adrenal gland, skin, muscle, lung, gastrointestinal tract (e.g., large intestine and small intestine), blood vessel, heart, thymus, spleen, submandibular gland, peripheral blood, prostate, testicle, ovary, placenta, uterus, bone, joint, skeletal muscle, and the like, and may also be a chemically synthesized protein or a protein synthesized using a cell-free translation system. Alternatively, the transcriptional regulatory factor of the present invention may be a recombinant protein produced by a transformant transferred with a polynucleotide having the base sequence that encodes the above-described amino acid sequence.

As substantially the same amino acid sequence as the amino acid sequence shown by SEQ ID NO:3, an amino acid sequence having a homology of about 70% or more, preferably about 80% or more, more preferably about 90% or more, particularly preferably about 95% or more, and most preferably about 98% or more, to the amino acid sequence shown by SEQ ID NO:3, and the like can be mentioned. Similarly, as substantially the same amino acid sequence as the amino acid sequence shown by SEQ ID NO:5, an amino acid sequence having a homology of about 70% or more, preferably about 80% or more, more preferably about 90% or more, particularly preferably about 95% or more, and most preferably about 98% or more, to the amino acid sequence shown by SEQ ID NO:5, and the like can be mentioned.

As examples of the protein that comprises substantially the same amino acid sequence as the amino acid sequence shown by SEQ ID NO:3 (or 5), a protein that comprises substantially the same amino acid sequence as the aforementioned amino acid sequence shown by SEQ ID NO:3 (or 5), and that has substantially the same quality of activity as a protein that comprises the amino acid sequence shown by SEQ ID NO:3 (or 5), and the like are preferred.

As examples of substantially the same quality of activity, an activity to bind to the FRE sequence of the present invention, transcription control activity on a gene under the control of a promoter comprising the sequence, and the like can be mentioned. Substantially the same quality means that the properties of the proteins are qualitatively (e.g., physiologically or pharmacologically) equivalent to each other. Accordingly, for example, it is preferable that the proteins be equivalent to each other in terms of transcription control activity (e.g., about 0.01 to 100 times, preferably about 0.1 to 10 times, more preferably 0.5 to 2 times), but quantitative factors such as the extent of activity and protein molecular weight may be different.

A measurement of transcription control activity can be conducted using a publicly known method, for example, Northern analysis of a target gene, gel shift assay and the like. Alternatively, the activity of the transcriptional regulatory factor of the present invention can also be evaluated using a method based on the intracellular localization thereof, for example, examining the degree of migration from cytoplasm to nucleus.

Examples of the NBP of the present invention (or the RBMX analogous protein: of the present invention) also include proteins comprising 1) an amino acid sequence having one or more amino acids (preferably about 1 to about 30, preferably about 1 to about 10, more preferably several (1 to 5) amino acids) deleted from the amino acid sequence shown by SEQ ID NO:3 (or 5), 2) an amino acid sequence having one or more amino acids (preferably about 1 to about 30, preferably about 1 to about 10, more preferably several (1 to 5) amino acids) added to the amino acid sequence shown by SEQ ID NO:3 (or 5), 3) an amino acid sequence having one or more amino acid (preferably about 1 to about 30, preferably about 1 to about 10, more preferably several (1 to 5) amino acids) inserted to the amino acid sequence shown by SEQ ID NO:3 (or 5), 4) an amino acid sequence having one or more amino acids (preferably about 1 to about 30, preferably about 1 to about 10, more preferably several (1 to 5) amino acids) substituted with other amino acids in the amino acid sequence shown by SEQ ID NO:3 (or 5), or 5) an amino acid sequence comprising a combination thereof.

When an amino acid sequence is inserted, deleted or substituted as described above, the position of the insertion, deletion or substitution is not subject to limitation, as long as the protein retains transcription control activity.

The NBP of the present invention is preferably a protein having the amino acid sequence shown by SEQ ID NO:3, that is, the mouse NBP or a homologue thereof in another mammal. In addition, the RBMX analogous protein of the present invention is preferably a protein having the amino acid sequence shown by SEQ ID NO:5, that is, the mouse RBMX analogous protein or a homologue thereof in another mammal.

For the proteins mentioned herein, the left end is the N terminal (amino terminal) and the right end is the C terminal (carboxyl terminal) in accordance with the conventional peptide marking. Regarding the transcriptional regulatory factor of: the present invention, the C terminal may be any of a carboxyl group (—COOH), a carboxylate (—COO⁻), an amide (—CONH₂), and an ester (—COOR). Here, as R in the ester, a C₁₋₆ alkyl group such as methyl, ethyl, n-propyl, isopropyl, and n butyl; a C₃₋₈ cycloalkyl group such as cyclopentyl and cyclohexyl; a C₆₋₁₂ aryl group such as phenyl and α-naphthyl; a phenyl-C₁₋₂ alkyl group such as benzyl and phenethyl; a C₇₋₁₄ aralkyl group such as an α-naphthyl-C₁₋₂ alkyl group such as α-naphthylmethyl; a pivaloyloxymethyl group; and the like are used.

When the transcriptional regulatory factor has a carboxyl group (or a carboxylate) at a position other than the C terminal, a protein wherein the carboxyl group is amidated or esterified is also included in the transcriptional regulatory factor of the present invention. In this case, as the ester, the above-described ester at the C terminal, and the like, for example, are used.

Furthermore, the transcriptional regulatory factor of the present invention also includes a protein wherein the amino group of the N terminal amino acid residue (e.g., methionine residue) is protected by a protecting group (for example, C₁₋₆ acyl groups such as C₁₋₆ alkanoyl groups such as formyl group and acetyl group, and the like), a protein wherein the N terminal glutamine residue, which is produced upon cleavage in vivo, has been converted to pyroglutamic acid, a protein wherein a substituent (for example, —OH, —SH, amino group, imidazole group, indole group, guanidino group, and the like) on a side chain of an amino acid in the molecule is protected by an appropriate protecting group (for example, C₁₋₆ acyl groups such as C₁₋₆ alkanoyl groups such as formyl group and acetyl group, and the like), a conjugated protein such as what is called a glycoprotein having a sugar chain bound thereto, and the like.

The present invention also provides a partial peptide of the above-described transcriptional regulatory factor of the present invention (hereinafter also simply abbreviated as a “partial peptide of the present invention”). The partial peptide may be any peptide as long as which have the above-described partial amino acid sequence of the transcriptional regulatory factor of the present invention, and have substantially the same quality of activity as the protein of the present invention; for example, the partial peptide is a peptide which comprises the DNA binding domain and the transcription regulatory (activation) domain of the transcriptional regulatory factor. Here, “substantially the same quality of activity” means an activity to bind to a DNA (FRE of the present invention) and transcription promoting activity on a gene under the control of the FRE.

The peptides comprising a partial amino acid sequence of the transcriptional regulatory factor of the present invention include those possessing an activity to bind to a DNA (FRE of the present invention), but not possessing transcription promoting activity on a gene under the control of the FRE (for example, those comprising the DNA-binding domain of the transcriptional regulatory factor, but not comprising the transcription regulatory (activation) domain), which, however, do not fall in the scope of “the partial peptide of the present invention”. However, because such a peptide is capable of binding to the FRE sequence of the present invention in an SREBP-1c promoter to block the transcriptional activation of the SREBP-1c gene by the transcriptional regulatory factor of the present invention, it is useful as a prophylactic or therapeutic drug for a metabolic disorder, especially for a glucose or lipid metabolic disorder as described below.

For the partial peptide of the present invention, the C terminal may be any of a carboxyl group (—COOH), a carboxylate (—COO⁻), an amide (—CONH₂), and an ester (—COOR). Here, as R in the ester, the same as those mentioned for the transcriptional regulatory factor of the present invention above can be mentioned. When the partial peptide of the present invention has a carboxyl group (or a carboxylate) at a position other than the C terminal, a partial peptide wherein the carboxyl group is amidated or esterified is also included in the partial peptide of the present invention. In this case, as the ester, the above-described ester at the C terminal, and the like, for example, are used.

Furthermore, the partial peptide of the present invention also includes a partial peptide wherein the amino group of the N terminal methionine residue is protected by a protecting group, a partial peptide wherein glutamine residue, which is produced upon cleavage at the N terminal in vivo, has been converted to pyroglutamic acid, a partial peptide wherein a substituent on a side chain of an amino acid in the molecule is protected by an appropriate protecting group, a conjugated peptide such as what is called a glycopeptide having a sugar chain bound thereto, and the like, as with the above-described transcriptional regulatory factor of the present invention.

The transcriptional regulatory factor of the present invention or a partial peptide thereof may be a free form or a form of salt. As the salt, physiologically acceptable salts with acid or base can be mentioned, and physiologically acceptable acid addition salts are particularly preferred. Useful salts include, for example, salts with inorganic acids (for example, hydrochloric acid, phosphoric acid, hydrobromic acid, sulfuric acid) or salts with organic acids (for example, acetic acid, formic acid, propionic acid, fumaric acid, maleic acid, succinic acid, tartaric acid, citric acid, malic acid, oxalic acid, benzoic acid, methanesulfonic acid, benzenesulfonic acid) and the like.

The transcriptional regulatory factor of the present invention can be produced from a cell or tissue of the aforementioned mammal by a method known per se of protein purification. Specifically, the transcriptional regulatory factor of the present invention can be produced by homogenizing mammalian tissue or cells, and separating and purifying the nuclear fraction by a chromatography such as reversed-phase chromatography, ion exchange chromatography or affinity chromatography, and the like.

Specifically, the transcriptional regulatory factor of the present invention can be obtained by bringing a DNA comprising the FRE base sequence of the present invention into contact with a cell nuclear extract derived from a human or another mammal (e.g., mouse, rat, rabbit, guinea pig, hamster, bovine, horse, swine, sheep, monkey, dog, cat and the like), preferably from a strain or individual showing a tendency for increased expression of SREBP-1c or a metabolic disorder due to meals (sugar loading), especially due to a high-fructose diet (fructose loading), and recovering (separating and purifying) the protein bound to the DNA. The animal cell for obtaining a nuclear extract is not subject to limitation, as long as it is a cell expressing the desired transcriptional regulatory factor; as such animal cells, the various cells described above in relation to the preparation of a nucleic acid comprising the FRE of the present invention, or tissues comprising such cells and the like, can be mentioned, with preference given to hepatocytes, more preferably sugar-loaded hepatocytes, particularly preferably fructose-loaded hepatocytes. These hepatocytes may be those obtained by incubating a culture of a cell line established from a liver-derived cell or a primary cell culture with the addition of a sugar (e.g., fructose) for a given time, or may be a liver tissue resected from an animal body given a diet (e.g., high-fructose diet).

Preparation of nuclear extract from a cell or a tissue is carried out by conventional methods. For example, such methods include a method wherein the nuclear extract is obtained by suspending the cell or the tissue in an appropriate buffer (e.g., phosphate buffer, PBS, Tris-HCl buffer, HEPES buffer and the like; the buffer may contain a protein denaturant such as urea or guanidine hydrochloride and a surfactant such as Triton X-100™), disrupting the cell by means of sonication, lysozyme and/or freeze-thawing and the like, subsequently treating a precipitate obtained by centrifugation or filtration with, for example, hypertonic solution and the like, and centrifuging the solution to recover a supernatant.

The means of contacting the nuclear extract and the DNA comprising the FRE base sequence of the present invention is not subject to limitation; for example, an affinity column with the DNA immobilized to an appropriate insoluble carrier (e.g., agarose, cellulose, Sepharose and the like) is prepared, the nuclear extract is passed through the column to bind the desired transcriptional regulatory factor and FRE, this is followed by elution using a density gradient of NaCl, KCl and the like, and a protein containing fraction eluted at a high salt concentration is recovered as a fraction containing a transcriptional regulatory factor capable of specifically binding to FRE.

Isolation and purification of the transcriptional regulatory factor of the present invention contained in the thus-obtained fraction can be conducted according to a method know per se. Useful methods include methods based on solubility, such as salting-out and solvent precipitation; methods based mainly on molecular weight differences, such as dialysis, ultrafiltration, gel filtration, and SDS-polyacrylamide gel electrophoresis; methods based on charge differences, such as ion exchange chromatography; methods based on specific affinity, such as affinity chromatography; methods based on hydrophobicity differences, such as reversed-phase high performance liquid chromatography; and methods based on isoelectric point differences, such as isoelectric focusing. These methods can be combined as appropriate.

The transcriptional regulatory factor of the present invention or a partial peptide thereof can also be produced by completely decomposing (decomposing with acid or alkali) the transcriptional regulatory factor as purified, examining its amino acid composition, the amino acid sequence of a partial peptide obtained by limited decomposition using a sequence-specific chemical substance such as peptidase or bromocyan is determined using a publicly known technique such as Edman degradation, the entire amino acid sequence is determined based on all information thus obtained, and thereafter the desired product is produced on the basis of the amino acid sequence according to a publicly known method of peptide synthesis.

The method of peptide synthesis may be any of, for example, a solid phase synthesis process and a liquid phase synthesis process. A desired transcriptional regulatory factor can be produced by condensing a partial peptide or amino acid capable of constituting the transcriptional regulatory factor of the present invention with the remaining portion, and removing any protecting group the resultant product may have.

Here, the condensation and the protecting group removal are conducted in accordance with methods known per se, for example, the methods indicated in 1) to 5) below:
1) M. Bodanszky and M. A. Ondetti: Peptide Synthesis, Interscience Publishers, New York (1966)
2) Schroeder and Luebke: The Peptide, Academic Press, New York (1965)
3) Nobuo Izumiya, et al.: Peptide Gosei-no-Kiso to Jikken (Basics and experiments of peptide synthesis), published by Maruzen Co. (1975)
4) Haruaki Yajima and Shunpei Sakakibara: Seikagaku Jikken Koza (Biochemical Experiment) 1, Tanpakushitsu no Kagaku (Chemistry of Proteins) IV, 205 (1977)
5) Haruaki Yajima, ed.: Zoku Iyakuhin no Kaihatsu (A sequel to Development of Pharmaceuticals), Vol. 14, Peptide Synthesis, published by Hirokawa Shoten.

The thus-obtained transcriptional regulatory factor can be purified and isolated by a publicly known method of purification. Here, as examples of the method of purification, solvent extraction, distillation, column chromatography, liquid chromatography, recrystallization, a combination thereof, and the like can be mentioned.

When the transcriptional regulatory factor obtained by the above-described method is a free form, it can be converted to an appropriate salt by a publicly known method or a method based thereon; conversely, when the protein is obtained in the form of a salt, the salt can be converted to a free form or another salt by a publicly known method or a method based thereon.

For the synthesis of the transcriptional regulatory factor of the present invention or a partial peptide thereof, an ordinary commercially available resin for protein synthesis can be used. As examples of such resins, chloromethyl resin, hydroxymethyl resin, benzhydrylamine resin, aminomethyl resin, 4-benzyloxybenzyl alcohol resin, 4-methylbenzhydrylamine resin, PAM resin, 4-hydroxymethylmethylphenylacetamidomethyl resin, polyacrylamide resin, 4-(2′,4′-dimethoxyphenyl-hydroxymethyl)phenoxy resin, 4-(2′,4′-dimethoxyphenyl-Fmoc-aminoethyl)phenoxy resin and the like can be mentioned. Using such a resin, an amino acid having an appropriately protected α-amino group and side chain functional group is condensed on the resin in accordance with the sequence of the desired transcriptional regulatory factor or a peptide thereof according to one of various methods of condensation known per se. At the end of the reaction, the protein (peptide) is cleaved from the resin and at the same time various protecting groups are removed, and a reaction to form an intramolecular disulfide bond is carried out in a highly diluted solution to obtain the desired protein (peptide) or an amide thereof.

For the above-described condensation of protected amino acids, various activation reagents which can be used for protein synthesis can be used, and a carbodiimide is preferably used. As the carbodiimide, DCC, N, N′-diisopropylcarbodiimide, N-ethyl-N′-(3-dimethylaminopropyl)carbodiimide and the like are used. For the activation using these carbodiimides, the protected amino acid, along with a racemation-suppressing additive (for example, HOBt, HOOBt), may be added directly to the resin, or the protected amino acid may be activated in advance as a symmetric acid anhydride or HOBt ester or HOOBt ester and then added to the resin.

Solvents used for the activation of protected amino acids and condensation thereof with a resin can be appropriately selected from among solvents that are known to be usable for protein condensation reactions. As examples of useful solvents, acid amides such as N,N-dimethylformamide, N,N-dimethylacetamide and N-methylpyrrolidone; halogenated hydrocarbons such as methylene chloride and chloroform; alcohols such as trifluoroethanol; sulfoxides such as dimethyl sulfoxide; amines such as pyridine; ethers such as dioxane and tetrahydrofuran; nitriles such as acetonitrile and propionitrile; esters such as methyl acetate and ethyl acetate; suitable mixtures thereof; and the like can be mentioned. Reaction temperature is appropriately selected from the range that is known to be usable for protein binding reactions, and is normally selected from the range of about −20° C. to about 50° C. An activated amino acid derivative is normally used from 1.5 to 4 times in excess. When a test using the ninhydrin reaction reveals that the condensation is insufficient, sufficient condensation can be conducted by repeating the condensation reaction without elimination of protecting groups. If the condensation is insufficient even though the reaction is repeated, unreacted amino acids may be acetylated using acetic anhydride or acetylimidazole.

A protecting method and a protecting group for a functional group that should not be involved in the reaction of raw materials, a method of removing the protecting group, a method of activating a functional group involved in the reaction, and the like can be appropriately selected from among publicly known groups or publicly known means.

As examples of the protecting group for an amino group of the raw material, Z, Boc, tertiary pentyloxycarbonyl, isobornyloxycarbonyl, 4-methoxybenzyloxycarbonyl, C1-Z, Br-Z, adamantyloxycarbonyl, trifluoroacetyl, phthaloyl, formyl, 2-nitrophenylsulfenyl, diphenylphosphinothioyl, Fmoc, and the like can be used.

A carboxyl group can be protected, for example, by alkyl esterification (for example, linear, branched or cyclic alkyl esterification with methyl, ethyl, propyl, butyl, tertiary butyl, cyclopentyl, cyclohexyl, cycloheptyl, cyclooctyl, 2-adamantyl, and the like), aralkyl esterification (for example, benzyl esterification, 4-nitrobenzyl esterification, 4-methoxybenzyl esterification, 4-chlorobenzyl esterification, benzhydryl esterification), phenacyl esterification, benzyloxycarbonyl hydrazidation, tertiary butoxycarbonyl hydrazidation, trityl hydrazidation, and the like.

The hydroxyl group of serine can be protected by, for example, esterification or etherification. As examples of a group suitable for this esterification, lower alkanoyl groups such as an acetyl group, aroyl groups such as a benzoyl group, and groups derived from carbonic acid such as a benzyloxycarbonyl group and an ethoxycarbonyl group, and the like are used. In addition, as examples of a group suitable for etherification, a benzyl group, a tetrahydropyranyl group, a t-butyl group, and the like can be mentioned.

As examples of the protecting group for the phenolic hydroxyl group of tyrosine, Bzl, Cl₂-Bzl, 2-nitrobenzyl, Br-Z, tertiary butyl, and the like can be used.

As examples of the protecting group for the imidazole of histidine, Tos, 4-methoxy-2,3,6-trimethylbenzenesulfonyl, DNP, benzyloxymethyl, Bum, Boc, Trt, Fmoc, and the like are used.

As examples of the method of removing (eliminating) a protecting group, catalytic reduction in a hydrogen stream in the presence of a catalyst such as Pd-black or Pd-carbon; acid treatment by means of anhydrous hydrogen fluoride, methanesulfonic acid, trifluoromethane-sulfonic acid, trifluoroacetic acid, or a mixture solution thereof; base treatment by means of diisopropylethylamine, triethylamine, piperidine, piperazine or the like; and reduction with sodium in liquid ammonia, and the like are used. The elimination reaction by the above-described acid treatment is generally carried out at a temperature of about −20° C. to about 40° C.; the acid treatment is efficiently conducted by adding a cation scavenger, for example, anisole, phenol, thioanisole, metacresol, paracresol, dimethylsulfide, 1,4-butanedithiol and 1,2-ethanedithiol. Also, a 2,4-dinitrophenyl group used as a protecting group of the imidazole of histidine is removed by thiophenol treatment; a formyl group used as a protecting group of the indole of tryptophan is removed by deprotection by acid treatment in the presence of 1,2-ethanedithiol, 1,4-butanedithiol, or the like, as well as by alkali treatment with a dilute sodium hydroxide solution, dilute ammonia, or the like.

As examples of those obtained by activation of the carboxyl group in the raw material, a corresponding acid anhydride, an azide, an activated ester [an ester with an alcohol (for example, pentachlorophenol, 2,4,5-trichlorophenol, 2,4-dinitrophenol, cyanomethyl alcohol, paranitrophenol, HONB, N-hydroxysuccimide, N-hydroxyphthalimide, or HOBt)] and the like are used. As examples of those obtained by activation of the amino group in the raw material, a corresponding phosphoric amide is used.

In another method of preparing an amide of the protein (peptide), for example, the α-carboxyl group of the carboxy terminal amino acid is first amidated and hence protected, and a peptide chain is elongated to a desired chain length toward the amino group side, thereafter the both proteins (peptides) having the protecting group for the N terminal α-amino group of the peptide chain only removed and the protein (peptide) having the protecting group for the C terminal carboxyl group only removed are prepared, and the protein (peptide) are condensed in a mixed solvent described above. For details about the condensation reaction, the same as above applies. After the protected protein (peptide) obtained by the condensation is purified, all protecting groups can be removed by the above-described method to yield a desired crude protein (peptide). By purifying this crude protein (peptide) using various publicly known means of purification, and freeze-drying the main fraction, a desired amide of the protein (peptide) can be prepared.

In order to obtain esters of the protein (peptide), a desired ester of the protein (peptide) can be prepared by, for example, condensing the α-carboxyl group of the carboxy terminal amino acid with a desired alcohol to yield an amino acid ester, and then treating the ester in the same manner as with an amide of the protein (peptide).

The partial peptide of the present invention can also be produced by cleaving the transcriptional regulatory factor of the present invention with an appropriate peptidase.

Furthermore, the transcriptional regulatory factor of the present invention or a partial peptide thereof can also be produced by cultivating a transformant comprising DNA that encodes the transcriptional regulatory factor of the present invention or a partial peptide thereof, and separating and purifying the transcriptional regulatory factor of the present invention or a partial peptide thereof from the culture obtained. Alternatively, the transcriptional regulatory factor of the present invention or a partial peptide thereof can also be synthesized by in vitro translation using a cell-free protein translation system that comprises a rabbit reticulocyte lysate, wheat germ lysate, Escherichia coli lysate and the like, with RNA corresponding to the DNA as the template. Alternatively, it can be synthesized using a cell-free transcription/translation system containing RNA polymerase, with the DNA as the template.

As the DNA that encodes the transcriptional regulatory factor of the present invention or a partial peptide thereof, genomic DNA, a genomic DNA library, cDNA derived from any cell (for example, splenocyte, nerve cell, glial cell, pancreatic β cell, myelocyte, mesangial cell, Langerhans' cell, epidermal cell, epithelial cell, endothelial cell, fibroblast, fibrocyte, myocyte, adipocyte, immune cell (for example, macrophage, T cell, B cell, natural killer cell, mast cell, neutrophil, basophil, eosinophil, monocyte), megakaryocyte, synovial cell, chondrocyte, bone cell, osteoblast, osteoclast, mammary gland cell, hepatocyte or interstitial cell, or corresponding precursor cell, stem cell or cancer cell thereof, and the like) of a human or another mammal (for example, bovine, monkey, horse, swine, sheep, goat, dog, cat, guinea pig, rat, mouse, rabbit, hamster, and the like), a blood cell system cell, or any tissue where such cells are present, for example, brain or any portion of the brain (e.g., olfactory bulb, amygdaloid nucleus, basal ganglia, hippocampus, thalamus, hypothalamus, subthalamic nucleus, cerebral cortex, medulla oblongata, cerebellum, occipital lobe, frontal lobe, temporal lobe, putamen, caudate nucleus, corpus callosum, substantia nigra), spinal cord, hypophysis, stomach, pancreas, kidney, liver, gonad, thyroid, gall-bladder, bone marrow, adrenal gland, skin, muscle, lung, gastrointestinal tract (e.g., large intestine, small intestine), blood vessel, heart, thymus, spleen, submandibular gland, peripheralsblood, peripheral blood cells, prostate, testicle, testis, ovary, placenta, uterus, bone, joint, skeletal muscle, and the like (particularly the brain or any portion of the brain), a cDNA library derived from the aforementioned cell or tissue, synthetic DNA and the like can be mentioned. The vector used for the library may be any of a bacteriophage, a plasmid, a cosmid, a phagemid and the like. The DNA can also be amplified directly by a reverse transcriptase polymerase chain reaction (hereinafter abbreviated as “RT-PCR method”) using a total RNA or mRNA fraction prepared from the above-described cell or tissue.

As examples of the DNA that encodes the NBP of the present invention, DNA comprising the base sequence shown by SEQ ID-NO:2, DNA that comprises-a base sequence hybridizing to the base sequence shown by SEQ ID NO:2 under highly stringent conditions, and that encodes the aforementioned protein having substantially the same quality of activity (e.g., transcription control activity and the like) as a protein comprising the amino acid sequence shown by SEQ ID NO:3, and the like can be mentioned.

As examples of the DNA that encodes the RBMX analogous protein of the present invention, DNA comprising the base sequence shown by SEQ ID NO:4, DNA that comprises a base sequence hybridizing to the base sequence shown by SEQ ID NO:4 under highly stringent conditions, and that encodes the aforementioned protein having substantially the same quality of activity (e.g., transcription control activity and the like) as a protein comprising the amino acid sequence shown by SEQ ID NO:5, and the like can be mentioned.

As examples of the DNA capable of hybridizing to the base sequence shown by SEQ ID NO:2 (or 4) under highly stringent conditions, DNA that comprises a base sequence showing a homology of about 50% or more, preferably about 60% or more, more preferably about 70% or more, particularly preferably about 80% or more, and most preferably about 90% or more, to the base sequence shown by SEQ ID NO:2 (or 4), and the like are used.

Hybridization can be conducted according to a method known per se or a method based thereon, for example, a method described in Molecular Cloning, 2nd edition (J. Sambrook et al., Cold Spring Harbor Lab. Press, 1989) and the like. When a commercially available library is used, hybridization can be conducted according to the method described in the instruction manual attached thereto. Hybridization can preferably be conducted under highly stringent conditions.

High-stringent conditions refer to, for example, conditions involving a sodium concentration of about 19 to 40 mM, preferably about 19 to 20 mM, and a temperature of about 50 to 70° C., preferably about 60 to 65° C. In particular, a case wherein the sodium concentration is about 19 mM and the temperature is about 65° C. is preferred.

The DNA that encodes the NBP of the present invention is preferably DNA comprising the base sequence shown by SEQ ID NO:2 and the like. Additionally, the DNA that encodes the RBMX analogous protein of the present invention is preferably DNA comprising the base sequence shown by SEQ ID NO:4 and the like.

The DNA that encodes the partial peptide of the NBP of the present invention (or the RBMX analogous protein of the present invention) may be any DNA comprising the base sequence that encodes the same or substantially the same amino acid sequence as a portion of the amino acid sequence shown by SEQ ID NO:3 (or 5), and encoding a peptide having substantially the same quality of activity (e.g., transcription control activity and the like) as a protein comprising the aforementioned amino acid sequence shown by SEQ ID NO:3 (or 5). The DNA may be any of genomic DNA, a genomic DNA library, cDNA derived from the above-described cell or tissue, a cDNA library derived from the above-described cell or tissue, and synthetic DNA. The vector used for the library may be any of a bacteriophage, a plasmid, a cosmid, a phagemid and the like. The DNA can also be amplified directly by the RT-PCR method using an mRNA fraction prepared from the above-described cell or tissue.

Specifically, as examples of the DNA that encodes the partial peptide of the NBP of the present invention (or the RBMX analogous protein of the present invention), (1) DNA that comprises a partial base sequence of DNA comprising the base sequence shown by SEQ ID NO:2 (or 4), (2) DNA that comprises a base sequence hybridizing to DNA comprising the base sequence shown by SEQ ID NO:2 (or 4) under highly stringent conditions, and that encodes a peptide having substantially the same quality of activity (e.g., transcription control activity and the like) as that of a protein comprising the amino acid sequence encoded by the DNA and the like are used.

As examples of the DNA capable of hybridizing to the base sequence shown by SEQ ID NO:2 (or 4) under highly stringent conditions, a polynucleotide comprising a base sequence showing a homology of about 60% or more, preferably about 70% or more, more preferably about 80% or more, and most preferably about 90% or more, to the base sequence, and the like are used.

The DNA that encodes the transcriptional regulatory factor of the present invention or a partial peptide thereof may be a free form or a form of salt. As the salt, physiologically acceptable salts with acid or base can be mentioned, and physiologically acceptable acid addition salts are particularly preferred. Useful salts include, for example, salts with inorganic acids (for example, hydrochloric acid, phosphoric acid, hydrobromic acid, sulfuric acid) or salts with organic acids (for example, acetic acid, formic acid, propionic acid, fumaric acid, maleic acid, succinic acid, tartaric acid, citric acid, malic acid, oxalic acid, benzoic acid, methanesulfonic acid, benzenesulfonic acid) and the like.

The DNA that encodes the transcriptional regulatory factor of the present invention or a partial peptide thereof can be cloned by amplifying it by the PCR method using a synthetic DNA primer comprising a portion of the base sequence that encodes the factor or the partial peptide, or by hybridizing DNA incorporated in an appropriate expression vector to a labeled DNA fragment or synthetic DNA that encodes a portion or the entire region of the protein of the present invention. Hybridization can be conducted according to, for example, a method described in Molecular Cloning, 2nd edition (ibidem) and the like. When a commercially available library is used, hybridization can be conducted according to the method described in the instruction manual attached to the library.

Preferably, a DNA that encodes the transcriptional regulatory factor of the present invention or a partial peptide thereof can be obtained by introducing into a host cell a reporter gene under the control of a promoter for the host cell comprising a DNA comprising the sequence of the fructose responsive element sequence of the present invention and an animal-derived cDNA library under the control of the promoter for the host cell, and isolating cDNA introduced to a cell that expresses the reporter gene at a high level. Although any promoter capable of exhibiting promoter activity in the host cell used can be used as the promoter for the host cell, a promoter capable of functioning in yeast cells is preferred; for example, PHO5 promoter, PGK promoter, GAP promoter, ADH promoter and the like are used. Introduction of the FRE sequence of the present invention to such a promoter (construction of chimeric promoter) can be conducted by combining gene engineering techniques known per se. As the reporter gene, any one known per se can be used; for example, luciferase gene, GFP gene, alkaline phosphatase gene, peroxidase gene, β-galactosidase gene and the like can be mentioned, but these are not to be construed as limiting.

Although the animal-derived CDNA library may be derived from a cell or tissue of any mammal (e.g., human, mouse, rat, guinea pig, hamster, rabbit, sheep, bovine, horse, swine, dog, cat, monkey and the like): that expresses the desired transcriptional regulatory factor, it is preferably a cDNA library derived from a strain or individual showing a tendency for increased expression of SREBP-1c or a metabolic disorder due to meals (sugar loading), especially to a high-fructose diet (fructose load), more preferably from a hepatocyte of the strain or individual, still more preferably from a sugar-loaded hepatocyte, and most preferably from a fructose-loaded hepatocyte. Each cDNA that constitutes the library can be cloned downstream of a promoter for the host cell suitable for the host cell used, using a gene engineering technique known per se. As the promoter, a promoter capable of functioning in yeast cells as described above is preferably used.

As transfer vectors carrying the above-described expression cassette, plasmids derived from Escherichia coli (e.g., pBR322, pBR325, pUC12, pUC13); plasmids derived from Bacillus subtilis (e.g., pUB110, pTP5, pC194); plasmids derived from yeast (e.g., pSH19, pSH15); bacteriophages such as λ phage; and the like, are used. Optionally, expression vectors that comprise another enhancer, a polyA addition signal, a selection marker, an SV40 replication origin (hereinafter also abbreviated as SV40ori) and the like can be used. The selection marker includes, for example, the dihydrofolate reductase gene [methotrexate (MTX) resistance], the ampicillin resistance gene, the neomycin resistance gene (G418 resistance) and the like, as well as genes complementing auxotrophic (leucine-auxotrophic, tryptophan-auxotrophic, etc.) mutation and the like.

As useful examples of the host, yeast, an insect cell, a mammal cell and the like can be mentioned. Preferably, the host include a yeast cell for the purpose of avoiding background due to transcriptional regulatory factor existed within the host. Specifically, as useful examples of the yeast, Saccharomyces cerevisiae AH22, AH22R⁻, NA87-11A, DKD-5D and 20B-12, Schizosaccharomyces pombe NCYC1913 and NCYC2036, Pichia pastoris KM71 and the like can be mentioned.

Transformation can be carried out according to a method known per se, depending on a kind of host. For example, when the host is yeast cell, for example, yeast cell can be transformed in accordance with a method described in Methods in Enzymology, Vol. 194, 182-187 (1991), Proc. Natl. Acad. Sci. USA, Vol. 75, 1929 (1978), and the like.

Cultivation of transformant can be carried out according to a method known per se, depending on a kind of host. As examples of the medium for cultivating a transformant whose host is a yeast, Burkholder's minimum medium [Bostian, K. L. et al., Proc. Natl. Acad. Sci. USA, vol. 77, 4505 (1980)] and SD medium supplemented with 0.5% casamino acid [Bitter, G. A. et al., Proc. Natl. Acad. Sci. USA, vol. 81, 5330 (1984)] can be mentioned. The medium's pH is preferably about 5 to 8. Cultivation is normally carried out at about 20° C. to about 35° C. for about 24 to about 72 hours. As necessary, the culture may be aerated or agitated.

After the transformant is cultured for a given time, the expression of the reporter gene is examined, and cDNA transferred to a transformant showing a significantly increased expression amount compared to control cells (host cells incorporating only an expression vector comprising the reporter gene) is cloned by a conventional method, whereby a DNA that encodes the transcriptional regulatory factor of the present invention or a partial peptide thereof that retains the DNA binding characteristic and transcription promoting activity can be obtained.

The base sequence of DNA can be converted according to a method known per se, such as the ODA-LA PCR method, the Gapped duplex method, the Kunkel method and the like, or a method based thereon, using a publicly known kit, for example, Mutan™-super Express Km (Takara Shuzo Co., Ltd.), Mutan™-K (Takara Shuzo Co., Ltd.) and the like.

The cloned DNA can be used as is, or after digestion with a restriction enzyme or addition of a linker as desired, depending on the purpose of its use. The DNA may have the translation initiation codon ATG at the 5′ end thereof, and the translation stop codon TAA, TGA or TAG at the 3′ end thereof. These translation initiation codons and translation stop codons can be added using an appropriate synthetic DNA adapter.

A DNA expression vector that encodes the transcriptional regulatory factor of the present invention can be produced by, for example, cutting out a desired DNA fragment from the DNA that encodes the transcriptional regulatory factor of the present invention, and joining the DNA fragment downstream of a promoter in an appropriate expression vector.

Useful expression vectors include plasmids derived from Escherichia coli (e.g., pBR322, pBR325, pUC12, pUC13); plasmids derived from Bacillus subtilis (e.g., pUB110, pTP5, pC194); plasmids derived from yeast (e.g., pSH19, pSH15); bacteriophages such as λ phage; animal viruses such as retrovirus, vaccinia virus and baculovirus; pA1-11, pXT1, pRc/CMV, pRc/RSV, pcDNAI/Neo, and the like.

The promoter may be any promoter, as long as it is appropriate for the host used to express the gene.

For example, when the host is an animal cell, the SRα romoter, the SV40 promoter, the LTR promoter, the CMV (cytomegalovirus) promoter, the HSV-TK promoter and the like are used. Of these, the CMV promoter, the SRα promoter and the like are preferred.

When the host is a bacterium of the genus Escherichia, the trp promoter, the lac promoter, the recA promoter, the γP_{L} promoter, the lpp promoter, the T7 promoter and the like are preferred.

When the host is a bacterium of the genus Bacillus, the SPO1 promoter, the SPO2 promoter, the penP promoter and the like are preferred.

When the host is yeast, the PHO5 promoter, the PGK promoter, the GAP promoter, the ADH promoter and the like are preferred.

When the host is an insect cell, the polyhedrin promoter, the P10 promoter and the like are preferred.

Useful expression vectors include, in addition to the above, those optionally harboring an enhancer, a splicing signal, a polyA addition signal, a selection marker, an SV40 replication origin (hereinafter also abbreviated as SV40ori) and the like. As examples of the selection marker, the dihydrofolate reductase (hereinafter also abbreviated as dhfr) gene [methotrexate (MTX) resistance], the ampicillin resistance gene (hereinafter also abbreviated as Amp^{r}), the neomycin resistance gene (hereinafter also abbreviated as Neo^{r}, G418 resistance) and the like can be mentioned. In particular, when a Chinese hamster cell lacking the dhfr gene is used in combination with the dhfr gene as the selection marker, a target gene can also be selected using a thymidine-free medium.

In addition, as required, a signal sequence that matches the host may be added to the N terminal side of the protein of the present invention. Useful signal sequences include a PhoA signal sequence, an OmpA signal sequence and the like when the host is a bacterium of the genus Escherichia; an α-amylase signal sequence, a subtilisin signal sequence and the like when the host is a bacterium of the genus Bacillus; an MFα signal sequence, an SUC2 signal sequence and the like when the host is yeast; and an insulin signal sequence, an α-interferon signal sequence, an antibody molecule signal sequence and the like when the host is an animal cell.

A transformant comprising the thus-obtained “DNA that encodes the transcriptional regulatory factor of the present invention” can be produced by transforming the host with an expression vector comprising the DNA according to a publicly known method.

Here, as the expression vector, those mentioned above can be mentioned.

Useful hosts include, for example, a bacterium of the genus Escherichia, a bacterium of the genus Bacillus, yeast, an insect cell, an insect, an animal cell and the like.

Useful bacteria of the genus Escherichia include, for example, Escherichia coli K12 DH1 (Proc. Natl. Acad. Sci. U.S.A., Vol. 60, 160 (1968)), JM103 (Nucleic Acids Research, Vol. 9, 309 (1981)), JA221 (Journal of Molecular Biology, Vol. 120, 517 (1978)), HB101 (Journal of Molecular Biology, Vol. 41, 459 (1969)), C600 (Genetics, Vol. 39, 440 (1954)) and the like.

Useful bacteria of the genus Bacillus include, for example, Bacillus subtilis MI114 (Gene, Vol. 24, 255 (1983)), 207-21 (Journal of Biochemistry, Vol. 95, 87 (1984)) and the like.

Useful yeasts include, for example, Saccharomyces cerevisiae AH22, AH22R, NA87-11A, DKD-5D and 20B-12, Schizosaccharomyces pombe NCYC1913 and NCYC2036, Pichia pastoris KM71, and the like.

Useful insect cells include, for example, Spodoptera frugiperda cell (Sf cell), MG1 cell derived from the mid-intestine of Trichoplusia ni, High Five™ cell derived from an egg of Trichoplusia ni, cell derived from Mamestra brassicae, cell derived from Estigmena acrea, and the like can be mentioned when the virus is AcNPV. When the virus is BmNPV, useful insect cells include Bombyx mori N cell (BmN cell) and the like. Useful Sf cells include, for example, Sf9 cell (ATCC CRL1711), Sf21 cell (both in Vaughn, J. L. et al., In Vivo, 13, 213-217 (1977) and the like.

Useful insects include, for example, a larva of Bombyx mori (Maeda et al., Nature, Vol. 315, 592 (1985)) and the like.

Useful animal cells include, for example, monkey cell COS-7, Vero, Chinese hamster cell CHO (hereafter abbreviated as CHO cell), Chinese hamster cell lacking the dhfr gene CHO (hereafter abbreviated as CHO(dhfr⁻) cell), mouse L cell, mouse AtT-20, mouse myeloma cell, rat GH3 human FL cell and the like.

Transformation can be carried out according to a method known per se, depending on a kind of host.

A bacterium of the genus Escherichia can be transformed, for example, according to a method described in Proc. Natl. Acad. Sci. U.S.A., Vol. 69, 2110 (1972), Gene, Vol. 17, 107 (1982) and the like.

A bacterium of the genus Bacillus can be transformed, for example, according to a method described in Molecular and General Genetics, Vol. 168, 111 (1979) and the like.

Yeast can be transformed, for example, according to a method described in Methods in Enzymology, Vol. 194, 182-187 (1991), Proc. Natl. Acad. Sci. USA, Vol. 75, 1929 (1978) and the like.

An insect cell and an insect can be transformed, for example, according to a method described in Bio/Technology, 6, 47-55 (1988) and the like.

An animal cell can be transformed, for example, according to a method described in Saibo Kogaku (Cell Engineering), extra issue 8, Shin Saibo Kogaku Jikken Protocol (New Cell Engineering Experimental Protocol), 263-267 (1995), published by Shujunsha, or Virology, Vol. 52, 456 (1973).

Cultivation of a transformant can be carried out according to a method known per se, depending on a kind of host.

For example, when a transformant whose host is a bacterium of the genus Escherichia or the genus Bacillus is cultivated, the culture medium is preferably a liquid medium. Also, the medium preferably contains a carbon source, a nitrogen source, an inorganic substance and the like necessary for the growth of the transformant. Here, as examples of the carbon source, glucose, dextrin, soluble starch, sucrose and the like can be mentioned; as examples of the nitrogen source, inorganic or organic substances such as an ammonium salt, a nitrate salt, corn steep liquor, peptone, casein, meat extract, soybean cake, potato extract and the like can be mentioned; as examples of the inorganic substance, calcium chloride, sodium dihydrogen phosphate, magnesium chloride and the like can be mentioned. In addition, the medium may be supplemented with yeast extract, vitamins, growth promoting factor and the like. Preferably, the pH of the medium is about 5 to about 8.

As an example of the medium used to cultivate a transformant whose host is a bacterium of the genus Escherichia, a M9 medium supplemented with glucose and a casamino acid (Miller, Journal of Experiments in Molecular Genetics, 431-433, Cold Spring Harbor Laboratory, New York, 1972) can be preferably mentioned. As required, in order to increase promoter efficiency, a chemical agent such as 3β-indolylacrylic acid may be added to the medium.

Cultivation of a transformant whose host is a bacterium of the genus Escherichia is normally carried out at about 15° C. to about 43° C. for about 3 to about 24 hours. As necessary, the culture may be aerated or agitated.

Cultivation of a transformant whose host is a bacterium of the genus Bacillus is normally carried out at about 30° C. to about 40° C. for about 6 to about 24 hours. As necessary, the culture may be aerated or agitated.

As examples of the medium for cultivating a transformant whose host is a yeast, Burkholder's minimum medium [Bostian, K. L. et al., Proc. Natl. Acad. Sci. USA, vol. 77, 4505 (1980)], SD medium supplemented with 0.5% casamino acid [Bitter, G. A. et al., Proc. Natl. Acad. Sci. USA, vol. 81, 5330 (1984)] and the like can be mentioned. The pH of the medium is preferably about 5 to 8. Cultivation is normally carried out at about 20° C. to about 35° C. for about 24 to about 72 hours. As necessary, the culture may be aerated or agitated.

Useful medium for cultivating a transformant whose host is an insect cell or an insect include, for example, Grace's insect medium [Grace, T. C. C., Nature, 195, 788 (1962)] supplemented with additives such as inactivated 10% bovine serum as appropriate and the like. The pH of the medium is preferably about 6.2 to 6.4. Cultivation is normally carried out at about 27° C. for about 3 to 5 days. As necessary, the culture may be aerated or agitated.

Useful medium for cultivating a transformant whose host is an animal cell include, for example, MEM medium supplemented with about 5 to 20% fetal bovine serum [Science, Vol. 122, 501(1952)], DMEM medium [Virology, Vol. 8, 396(1959)], RPMI 1640 medium [The Journal of the American Medical Association, Vol. 199, 519(1967)], 199 medium [Proceeding of the Society for the Biological Medicine, Vol. 73, 1(1950)] and the like. The pH of the medium is preferably about 6 to 8. Cultivation is normally carried out at about 30° C. to 40° C. for about 15 to 60 hours. As necessary, the culture may be aerated or agitated.

As described above, the transcriptional regulatory factor of the present invention can be produced in a cell (in the nucleus or in cytoplasm) of the transformant or outside the cell.

The transcriptional regulatory factor of the present invention or a partial peptide thereof can be separated and purified from the culture obtained by cultivating the aforementioned transformant according to a method known per se.

For example, when the transcriptional regulatory factor of the present invention or the partial peptide of the present invention is extracted from a cultured bacterium or a cell cytoplasm, a method is used as appropriate wherein bacteria or cells are collected from the culture by a known means, suspended in an appropriate buffer, and disrupted by means of sonication, lysozyme and/or freeze-thawing and the like, after which a crude extract of soluble protein is obtained by centrifugation or filtration. The buffer may contain a protein denaturant such as urea or guanidine hydrochloride and a surfactant such as Triton X-100™. On the other hand, when the transcriptional regulatory factor of the present invention or the partial peptide of the present invention is extracted from a nuclear fraction, a method of preparing a crude extract of the nuclear protein by treating the precipitate from the above-described centrifugation or filtration with, for example, a hypertonic solution and the like, and recovering the supernatant via centrifugation, and the like are used.

Isolation and purification of the transcriptional regulatory factor of the present invention or the partial peptide of the present invention contained in the thus-obtained soluble fraction or nuclear extract can be conducted according to a method know per se. Useful methods include methods based on solubility, such as salting-out and solvent precipitation; methods based mainly on molecular weight differences, such as dialysis, ultrafiltration, gel filtration, and SDS-polyacrylamide gel electrophoresis; methods based on charge differences, such as ion exchange chromatography; methods based on specific affinity, such as affinity chromatography; methods based on hydrophobicity differences, such as reversed-phase high performance liquid chromatography; and methods based on isoelectric point differences, such as isoelectric focusing. These methods can be combined as appropriate.

When the thus-obtained protein or peptide is a free form, it can be converted to a salt by a method known per se or a method based thereon; when the protein or peptide is obtained as a salt, it can be converted to a free form or another salt by a method known per se or a method based thereon.

Note that the protein or the peptide produced by the transformant can also be optionally modified by the action of an appropriate protein-modifying enzyme, before or after purification, or can have a polypeptide thereof removed partially. As such, useful protein-modifying enzymes include, for example, trypsin, chymotrypsin, arginyl endopeptidase, protein kinase, glycosidase and the like.

The presence of the thus-obtained protein of the present invention or partial peptide of the present invention can be confirmed by enzyme immunoassay, Western blotting and the like using a specific antibody.

Furthermore, the transcriptional regulatory factor of the present invention or the partial peptide of the present invention can also be synthesized by in vitro translation using a cell-free protein (transcription/) translation system including a rabbit reticulocyte lysate, wheat germ lysate, Escherichia coli lysate and the like, with RNA corresponding to the above-described DNA that encodes the transcriptional regulatory factor of the present invention or a partial peptide thereof as the template, as described above. The cell-free protein (transcription/) translation system may be a commercial product, or may be prepared by a method known per se; specifically, an Escherichia coli extract may be prepared in accordance with the method described in Pratt J. M et al., Transcription and Translation, 179-209, Hames B. D. & Higgins S. J. eds., IRL Press, Oxford (1984). As commercially available cell lysates, Escherichia coli-derived cell lysates such as the E. coli S30 extract system (manufactured by Promega) and the RTS 500 Rapid Translation System (manufactured by Roche) can be mentioned, rabbit reticulocyte-derived cell lysates such as the Rabbit Reticulocyte Lysate System (manufactured by Promega) can be mentioned, and wheat germ-derived cell lysates such as PROTEIOS™ (manufactured by TOYOBO) can be mentioned. Of these, use of a wheat germ lysate is preferred. As examples of methods of preparing a wheat germ lysate, the methods described in Johnston F. B. et al., Nature, 179, 160-161 (1957), Erickson A. H. et al., Meth. Enzymol., 96, 38-50 (1996), and the like can be used.

As a system or apparatus for protein synthesis, a batch method (Pratt, J. M. et al. (1984), ibidem), a continuous cell-free protein synthesis system wherein amino acids, energy sources and the like are continuously supplied to the reaction system (Spirin A. S. et al., Science, 242, 1162-1164 (1988)), the dialysis method (Kikawa et al., 21st general assembly of the Molecular Biology Society of Japan, WID6), or the overlay method (instruction manual of the PROTEIOS™ Wheat germ cell-free protein synthesis core kit: manufactured by TOYOBO) and the like can be mentioned. Furthermore, a method wherein template RNA, amino acids, energy sources and the like are supplied as required to the synthetic reaction system, and synthetic products and decomposition products are discharged whenever necessary (Japanese Patent Publication No. 2000-333673) and the like can be used.

The present invention also provides a screening method for a prophylactic or therapeutic substance for a metabolic disorder, especially glucose or lipid metabolic disorder (e.g., hypertriglyceridemia, hyper-LDL-cholesteremia, hypo-HDL-cholesterolemia, obesity, abnormality of glucose tolerance, fasting blood glucose disorder, hyperinsulinemia, hypertension, albuminuria, and the like), which comprises using the fructose responsive element of the present invention or a DNA comprising the same (may comprise the entire base sequence shown by SEQ ID NO:1; hereinafter also referred to as “the DNA of the present invention”) and the transcriptional regulatory factor of the present invention capable of binding to the element or a partial peptide thereof (hereinafter also simply referred to as “the transcriptional regulatory factor of the present invention”); As examples of specific embodiment of the screening method,
1) a method of detecting the inhibition of the binding of the DNA of the present invention and the transcriptional regulatory factor of the present invention in the presence of a test substance;
2) a method of comparing the expression of the gene between in the presence and in the absence of a test substance in an animal cell having a gene under the control of a promoter comprising the DNA of the present invention; and the like can be mentioned. In method 2) above, it is sometimes possible to increase the measurement sensitivity by loading a sugar on the animal cell.

When using the binding of the DNA of the present invention and the transcriptional regulatory factor of the present invention as an index, the screening method can be conducted by, for example, incubating the DNA of the present invention, previously labeled (e.g., ³²P, digoxigenin and the like), and the transcriptional regulatory factor of the present invention (may be either of the NBP of the present invention (including a partial peptide thereof) and the RBMX analogous protein of the present invention (including a partial peptide thereof), or both) in the presence of a test substance, thereafter the reactant is subjected to non-denatured gel electrophoresis to detect the disappearance or signal intensity reduction of a band corresponding to the DNA-transcriptional regulatory factor complex. Here, the transcriptional regulatory factor of the present invention may be used in the isolated and purified form, or in the form of a nuclear extract of cells that express the transcriptional regulatory factor. As examples of such cells, cells derived from a human or another mammalian individual having the expression of SREBP-1c increased due to sugar (e.g., fructose) loading, preferably hepatocytes, more preferably sugar-loaded hepatocytes, still more preferably fructose-loaded hepatocytes, and particularly preferably hepatocytes derived from a fructose-loaded CBA or C3H mouse, can be mentioned. Isolation of the nuclear extract from the cells can be conducted according to the above-described method.

As examples of the test compound, a peptide, a protein, a non-peptide compound, a synthetic compound, a fermentation product, a cell extract, a plant extract, an animal tissue extract and the like can be mentioned.

For example, if the signal intensity of the band corresponding to the DNA-transcriptional regulatory factor complex has decreased by about 20% or more, preferably 30% or more, more preferably about 50% or more, in the presence of a test substance, the test substance can be selected as an inhibitor of the DNA-binding activity of the transcriptional regulatory factor of the present invention.

When using the expression of a gene under the control of a promoter comprising the DNA of the present invention as an index, any promoter capable of functioning in animal cells can be used; for example, SRα promoter, SV40 promoter, LTR promoter, CMV (cytomegalovirus) promoter, HSV-TK promoter and the like are used. The DNA of the present invention can be inserted to an appropriate position in the promoter using a gene engineering technique known per se. Alternatively, an SREBP-1c promoter comprising the FRE of the present invention may be used as a “promoter comprising the DNA of the present invention”.

Although the gene under the control of a promoter comprising the DNA of the present invention is not subject to limitation, as long as it permits easy measurement of the expression amount thereof, reporter genes of luciferase, GFP, alkaline phosphatase, peroxidase, β-galactosidase and the like are preferably used. An SREBP-1c gene comprising an SREBP-1c promoter comprising the FRE of the present invention can also be used as a “gene under the control of a promoter comprising the DNA of the present invention”. In this case, a cell or tissue derived from a mammal that inherently has the SREBP-1c gene (e.g., CBA and C3H mice and the like) or the animal individual (excluding humans) can be used as an “animal cell comprising a gene under the control of a promoter comprising the DNA of the present invention”. When using a reporter gene as a gene under the control of a promoter comprising the DNA of the present invention, a reporter gene joined downstream of a promoter comprising the above-described DNA of the present invention using a gene engineering technique known per se can be inserted to an appropriate transfer vector, for example, a vector of an Escherichia coli-derived plasmid (e.g., pBR322, pBR325, pUC12, pUC13); a Bacillus subtilis-derived plasmid (e.g., pUB110, pTP5, pC194); a yeast-derived plasmid (e.g., pSH19, pSH15); a bacteriophages such as phage, and the like, and transferred to a host animal cell. The transfer vector may comprise another enhancer, polyA addition signal, selection marker, SV40 replication origin (hereinafter also abbreviated as SV40ori) and the like as required. As examples of the selection marker, dihydrofolate reductase gene [methotrexate (MTX) resistance], ampicillin resistance gene, neomycin resistance gene (G418 resistance) and the like can be mentioned.

The animal cell is not subject to limitation, as long as it is a cell capable of expressing the transcriptional regulatory factor of the present invention (preferably, a cell capable of expressing the factor in response to sugar loading); for example, various cell lines such as monkey cell COS-7, Vero, Chinese hamster ovarian cell (hereinafter abbreviated as CHO cell), Chinese hamster ovarian cell lacking the dhfr gene (hereinafter abbreviated as CHO (dhfr⁻) cell), mouse L cell, mouse AtT-20, mouse myeloma cell, rat GH3, human FL cell and the like can be used, and preferably hepatocytes, particularly preferably hepatocytes derived from CBA or C3H mice can be mentioned. These animal cells can be transformed, for example, according to a method described in Saibo Kogaku (Cell Engineering), extra issue 8, Shin Saibo Kogaku Jikken Protocol (New Cell Engineering Experimental Protocol), 263-267 (1995), published by Shujunsha, or Virology, Vol. 52, 456 (1973).

When a sugar is loaded on animal cell, the sugar loaded is not subject to limitation, as long as it is a carbohydrate that serves as an energy source, and monosaccharides such as glucose and fructose, disaccharides such as maltose, sucrose, and lactose, polysaccharides such as starch and glycogen, or mixtures thereof, and the like can be mentioned, preferably fructose or a mixture of fructose and other sugars. Sugar loading is conducted by the addition of a sugar to the culture; when using a non-human animal individual that inherently has an SREBP-1c gene comprising an SREBP-1c promoter comprising the above-described FRE of the present invention, sugar loading can be conducted by feeding the animal with a diet in common use for rearing the animal, a high-fructose diet known per se, and the like.

As examples of the test compound, a peptide, a protein, a non-peptide compound, a synthetic compound, a fermentation product, a cell extract, a plant extract, an animal tissue extract and the like can be mentioned.

A sugar is loaded in the presence and absence of a test substance, and cells are cultured for a given time in an appropriate medium (e.g., minimal essential medium, Dulbecco's odified Eagle medium, Ham medium, F12 medium, RPMI1680 medium, William's E medium and the like); thereafter, the expression of a gene under the control of a promoter comprising the DNA of the present invention is compared under the two conditions. When using the above-described non-human animal individual, feeding is conducted after a test substance is orally or non-orally (e.g., intravenously, intraperitoneally, intramuscularly, subcutaneously, intracutaneously and the like) administered; after the elapse of a given time, an appropriate biological sample (e.g., hepatocyte, blood and the like) is collected from the animal, the expression of the SREBP-1c gene is detected, and a comparison is made with individuals not receiving the test substance. The expression of the SREBP-1c gene can be detected and quantified by an immunoassay method such as ELISA using an anti-SREBP-1c antibody prepared by a conventional method, or the RT-PCR method.

As a result, for example, a test substance that inhibited the expression of the gene by about 20% or more, preferably 30% or more, more preferably about 50% or more, as an inhibitor of the transcription promoting activity on the transcriptional regulatory factor of the present invention.

Alternatively, the prophylactic or therapeutic substance for metabolic disorder (especially glucose or lipid metabolic disorder) can be screened by comparing the intracellular localization of the transcriptional regulatory factor of the present invention, for example, the degree of migration of the factor from cytoplasm to nucleus in the presence and absence of the test compound using animal cell which can be used in the above-described methods. More specifically, by immunostaining the cell with a fluorescently labeled antibody against the transcriptional regulatory factor of the present invention, for example, the migration of the factor from cytoplasm to nucleus can be monitored. Alternatively, it is also possible to directly monitor the migration of the transcriptional regulatory factor of the present invention from cytoplasm to nucleus using a transformant capable of expressing the factor in the form of a fusion protein with a fluorescent protein such as GFP (see, for example, Biochem. Biophys. Res. Commun., 278: 659-664 (2000)).

The “inhibitor of the transcriptional regulatory factor of the present invention” obtained by the above-described screening method is useful as a prophylactic or therapeutic substance for a metabolic disorder involved in an abnormality of the expression of the gene, especially glucose or lipid metabolic disorder (e.g., hypertriglyceridemia, hyper-LDL-cholesteremia, hypo-HDL-cholesterolemia, obesity, abnormality of glucose tolerance, fasting blood glucose disorder, hyperinsulinemia, hypertension, albuminuria, and the like), because it is capable of suppressing the increase in the expression of the gene due to a food (especially high-fructose diet) in a mammal having an SREBP-1c gene comprising the FRE of the present invention in the promoter region thereof.

Accordingly, the inhibitor of the transcriptional regulatory factor of the present invention (these substances may be any of a peptide, a protein, a non-peptide compound, a synthetic compound, a fermentation product, a cell extract, a plant extract, an animal tissue extract, plasma and the like, and also have formed a salt. Specific examples of the salt include the same salts as the aforementioned salts of transcriptional regulatory factor of the present invention) can be used as a prophylactic or therapeutic agent for a metabolic disorder after being mixed with a pharmacologically acceptable carrier into a pharmaceutical composition as necessary. Here, as the pharmacologically acceptable carrier, various organic or inorganic carrier substances conventionally used as pharmaceutical preparation materials can be used, and these are formulated as excipients, lubricants, binders and disintegrants, in solid preparations; as solvents, solubilizing agents, suspending agents, isotonizing agents, buffering agents and soothing agents, in liquid preparations, and the like. Also, as necessary, pharmaceutical preparation additives such as antiseptics, antioxidants, coloring agents, sweeteners and the like can be used.

As examples of suitable excipients, lactose, saccharose, D-mannitol, D-sorbitol, starch, gelatinized starch, dextrin, crystalline cellulose, low substituted hydroxypropyl cellulose, sodium carboxymethyl cellulose, gum arabic, pullulan, light silicic anhydride, synthetic aluminum silicate, magnesium metasilicate aluminate and the like can be mentioned.

As examples of suitable lubricants, magnesium stearate, calcium stearate, talc, colloidal silica and the like can be mentioned.

As examples of suitable binders, gelatinized starch, sucrose, gelatin, gum arabic, methyl cellulose, carboxymethyl cellulose, sodium carboxymethyl cellulose, crystalline cellulose, saccharose, D-mannitol, trehalose, dextrin, pullulan, hydroxypropyl cellulose, hydroxypropylmethyl cellulose, polyvinyl pyrrolidone and the like can be mentioned.

As examples of suitable disintegrants, lactose, saccharose, starch, carboxymethyl cellulose, calcium carboxymethyl cellulose, sodium crosscarmellose, sodium carboxymethyl starch, light silicic anhydride, low substituted hydroxypropyl cellulose and the like can be mentioned.

As examples of suitable solvents, water for injection, physiological saline, Ringer's solutions, alcohols, propylene glycol, polyethylene glycol, sesame oil, corn oil, olive oil, cottonseed oil and the like can be mentioned.

As examples of suitable solubilizing agents, polyethylene glycol, propylene glycol, D-mannitol, trehalose, benzyl benzoate, ethanol, trisaminomethane, cholesterol, triethanolamine, sodium carbonate, sodium citrate, sodium salicylate, sodium acetate and the like can be mentioned.

As examples of suitable suspending agents, surfactants such as stearyl triethanolamine, sodium lauryl sulfate, lauryl aminopropionic acid, lecithin, benzalkonium chloride, benzethonium chloride and glyceryl monostearate; hydrophilic polymers such as polyvinyl alcohol, polyvinyl pyrrolidone, sodium carboxymethyl cellulose, methyl cellulose, hydroxymethyl cellulose, hydroxyethyl cellulose and hydroxypropyl cellulose; polysorbates, polyoxyethylene hydrogenated castor oil and the like can be mentioned.

As examples of suitable isotonizing agents, sodium chloride, glycerin, D-mannitol, D-sorbitol, glucose and the like can be mentioned.

As examples of suitable buffers, buffers of a phosphate, an acetate, a carbonate, a citrate and the like, and the like can be mentioned.

As examples of suitable soothing agents, benzyl alcohol and the like can be mentioned.

As examples of suitable antiseptics, paraoxybenzoates, chlorobutanol, benzyl alcohol, phenethyl alcohol, dehydroacetic acid, sorbic acid and the like can be mentioned.

As examples of suitable antioxidants, sulfides, ascorbates and the like can be mentioned.

As examples of suitable coloring agents, water-soluble food tar colors (e.g., food colors such as Food Red Nos. 2 and 3, Food Yellow Nos. 4 and 5, and Food Blue Nos. 1 and 2), water-insoluble lake pigments (e.g., aluminum salts of the aforementioned water-soluble food tar colors and the like), natural pigments (e.g., β-carotene, chlorophyll, red iron oxide and the like) and the like can be mentioned.

As examples of suitable sweeteners, sodium saccharate, dipotassium glycyrrhizinate, aspartame, stevia and the like can be mentioned.

As examples of dosage forms of the aforementioned pharmaceutical composition, oral formulations such as tablets, capsules (including soft capsules and microcapsules), granules, powders, syrups, emulsions and suspensions; non-oral formulations such as injections (e.g., subcutaneous injections, intravenous injections, intramuscular injections, intraperitoneal injections and the like), external formulations (e.g., nasal preparations, transdermal preparations, ointments and the like), suppositories (e.g., rectal suppositories, vaginal suppositories and the like), pellets, drops, sustained-release preparations (e.g., sustained-release microcapsules and the like) and the like can be mentioned; these can be safely administered orally or non-orally.

The pharmaceutical composition can be produced by a method conventionally used in the field of pharmaceutical preparation making, for example, a method described in the Japanese Pharmacopoeia and the like. A specific method of producing a preparation is hereinafter described in detail. The content of the inhibitor of the transcriptional regulatory factor of the present invention in the pharmaceutical composition varies depending on the dosage form, the dose of the compound and the like; and is, for example, from about 0.1 to 100% by weight.

For example, an oral formulation is produced by adding to an active ingredient an excipient (e.g., lactose, saccharose, starch, D-mannitol and the like), a disintegrant (e.g., calcium carboxymethyl cellulose and the like), a binder (e.g., gelatinized starch, gum arabic, carboxymethyl cellulose, hydroxypropyl cellulose, polyvinyl pyrrolidone and the like), a lubricant (e.g., talc, magnesium stearate, polyethylene glycol 6000 and the like) and the like, compression-molding the resultant mixture, and subsequently, as required, coating the resulting material with a coating base by a method known per se for the purpose of taste masking, enteric solubility or sustained release.

As examples of the coating base, a sugar coating base, a water-soluble film coating base, an enteric film coating base, a sustained-release film coating base and the like can be mentioned.

As the sugar coating base, saccharose is used, and further which may be used in combination with one species or two or more species selected from among talc, precipitated calcium carbonate, gelatin, gum arabic, pullulan, carnauba wax and the like.

As examples of the water-soluble film coating base, cellulose polymers such as hydroxypropyl cellulose, hydroxypropylmethyl cellulose, hydroxyethyl cellulose and methylhydroxyethyl cellulose; synthetic polymers such as polyvinylacetal diethylanimoacetate, aminoalkylmethacrylate copolymer E [Eudragit-E (trade name), Rohm Pharma Corp.] and polyvinyl pyrrolidone; polysaccharides such as pullulan; and the like can be mentioned.

As examples of the enteric film coating base, cellulose polymers such as hydroxypropylmethyl cellulose phthalate, hydroxypropylmethyl cellulose acetate succinate, carboxymethylethyl cellulose, and cellulose acetate phthalate; acrylic polymers such as Methacrylic Acid Copolymer L [Eudragit-L (trade name), Rohm Pharma Corp.], Methacrylic Acid Copolymer LD [Eudragit-L-30D55 (trade name), Rohm Pharma Corp.], and Methacrylic Acid Copolymer S [Eudragit-S (trade name), Rohm Pharma Corp.]; natural substances such as shellac, and the like can be mentioned.

As examples of the sustained-release film coating base, cellulose polymers such as ethyl cellulose; acrylic polymers such as aminoalkyl methacrylate copolymer RS [Eudragit-RS (trade name), Rohm Pharma Corp.], and an ethyl acrylate-methylmethacrylate copolymer suspension [Eudragit-NE (trade name), Rohm Pharma Corp.]; and the like can be mentioned.

The above-mentioned coating bases may also be used in a mixture of two or more kinds thereof in a suitable ratio. Also, during coating, a shading agent, for example, titanium oxide, iron sesquioxide or the like, may be used.

An injection is produced by dissolving, suspending or emulsifying an active ingredient in an aqueous solvent (e.g., distilled water, physiological saline, Ringer's solution and the like), an oily solvent (e.g., vegetable oils such as olive oil, sesame oil, cottonseed oil and corn oil, propylene glycol, and the like), or the like, along with a dispersing agent (e.g., polysorbate 80, polyoxyethylene hydrogenated castor oil 60, polyethylene glycol, carboxymethyl cellulose, sodium alginate and the like), a preservative (e.g., methylparaben, propylparaben, benzyl alcohol, chlorobutanol, phenol and the like), an isotonizing agent (e.g., sodium chloride, glycerin, D-mannitol, D-sorbitol, glucose and the like), and the like. At this time, if desired, additives such as a solubilizing agent (e.g., sodium salicylate, sodium acetate and the like), a stabilizer (e.g., human serum albumin and the like), a soothing agent (e.g., benzyl alcohol and the like) and the like may also be used. An injection solution is normally packed in an appropriate ampoule.

Because the preparation thus obtained is safe and of low toxicity, it can be administered orally or non-orally to, for example, a mammal (for example, human, mouse, rat, rabbit, sheep, swine, bovine, horse, cat, dog, monkey, chimpanzee and the like).

The dosage of the prophylactic or therapeutic agent for metabolic disorder in accordance with the present invention varies depending on target disease, subject of administration, route of administration and the like; in an adult patient suffered from hypertriglyceridemia (body weight 60 kg), for example, the dosage is about 0.1 to 100 mg, preferably about 1.0 to 50 mg, more preferably about 1.0 to 20 mg, per day, based on the inhibitor of the transcriptional regulatory factor of the present invention, which is an active ingredient.

The present invention provides a prophylactic or therapeutic agent for metabolic disorder, especially glucose or lipid metabolic disorder (e.g., hypertriglyceridemia, hyper-LDL-cholesteremia, hypo-HDL-cholesterolemia, obesity, abnormality of glucose tolerance, fasting blood glucose disorder, hyperinsulinemia, hypertension, albuminuria, and the like), which comprises a substance that suppresses production or activity of the transcriptional regulatory factor of the present invention (it may be any one of, or both of the NBP of the present invention (including the partial peptide) and the RBMX analogous protein of the present invention (including the partial peptide)) (hereinafter, also referred to as a “substance that suppresses the production” or an “substance that suppresses the activity”).

Although the aforementioned prophylactic or therapeutic agent for a metabolic disorder containing a “substance that suppresses the production” or “substance that suppresses the activity” may be the “substance that suppresses the production” or “substance that suppresses the activity” as is, it is preferably a pharmaceutical composition prepared by mixing them with a pharmacologically acceptable carrier. Here, as the pharmacologically acceptable carrier, the same as those mentioned for the “inhibitor of the transcriptional regulatory factor of the present invention” obtained by the aforementioned screening method can be mentioned.

A pharmaceutical composition can be produced in the same manner as the aforementioned “inhibitor of the transcriptional regulatory factor of the present invention”.

Because the preparation thus obtained is safe and of low toxicity, it can be administered orally or non-orally to, for example, a mammal (for example, human, mouse, rat, rabbit, sheep, swine, bovine, horse, cat, dog, monkey, chimpanzee and the like).

The dosage of the prophylactic or therapeutic agent for metabolic disorder which comprises a “substance that suppresses the production” or an “substance that suppresses the activity”, varies depending on target disease, subject of administration, route of administration and the like; in an adult patient suffered from hypertriglyceridemia (body weight 60 kg), for example, the dosage is about 0.1 to 100 mg, preferably about 1.0 to 50 mg, more preferably about 1.0 to 20 mg, per day, based on the “substance that suppresses the production” or the “substance that suppresses the activity”, which is an active ingredient.

The substance that suppresses the activity may be any one, as long as it is capable of suppressing the activity of the transcriptional regulatory factor of the present invention, that is, the transcription promoting activity on a gene under the control of a promoter comprising the fructose responsive element of the present invention; for example, a substance that binds to the transcriptional regulatory factor of the present invention to inhibit the binding of the factor to the FRE sequence of the present invention (e.g., an antibody against the transcriptional regulatory factor of the present invention, a nucleic acid having a base sequence to which the transcriptional regulatory factor of the present invention can bind, and the like), a substance capable of promoting the decomposition/metabolism or inactivation of the transcriptional regulatory factor of the present invention (e.g., protease, protein modifying enzymes and the like) and the like can be mentioned.

Preferably, the substance that suppresses the activity includes an antibody against the transcriptional regulatory factor of the present invention or a partial peptide thereof or a salt thereof. An antibody against the transcriptional regulatory factor of the present invention or a partial peptide thereof or a salt thereof (hereinafter also abbreviated as an “antibody of the present invention”) can be produced according to a method known per se of producing an antibody or antiserum using the transcriptional regulatory factor (including the partial peptide and the salt) as an antigen. A monoclonal antibody or polyclonal antibody against the transcriptional regulatory factor of the present invention can be produced, for example, as described below.

[Preparation of Monoclonal Antibody]

(a) Preparation of monoclonal antibody-producing cells

The transcriptional regulatory factor of the present invention (the NBP of the present invention or the RBMX analogous protein of the present invention), as is or along with a carrier or a diluent, is administered to a mammal at a site permitting antibody production by administration. To increase antibody productivity in this administration, complete Freund's adjuvant and incomplete Freund's adjuvant may be administered. The administration is normally conducted every 2 to 6 weeks, in a total of about 2 to 10 times. As examples of the mammal used, monkey, rabbit, dog, guinea pig, mouse, rat, sheep and goat can be mentioned, and a mouse and a rat are preferably used.

For example, a monoclonal antibody-producing hybridoma can be prepared by selecting an individual with an antibody titer from among antigen-immunized-mammals, for example, mice, collecting the spleen or a lymph node 2-5 days after final immunization, and fusing an antibody-producing cell contained therein with an allogeneic or heterogeneous myeloma cell. A measurement of antibody titer in the antiserum can be conducted by, for example, reacting the labeled protein described below and an antiserum, and thereafter measuring the activity of the labeling agent bound to the antibody. The fusion procedure can be performed according to a known method, for example, the method of Köhler and Milstein [Nature, 256, 495 (1975)]. As examples of a fusogen, polyethylene glycol (PEG), Sendai virus and the like can be mentioned, and PEG is preferably used.

As examples of the myeloma cell, mammalian myeloma cells such as NS-1, P3U1, Sp2/O and AP-1 can be mentioned, and P3U1 is preferably used. A preferable ratio of the number of antibody-producing cells (splenocytes) and number of myeloma cells used is about 1:1 to 20:1; cell fusion can be efficiently erformed by adding a PEG (preferably PEG1000 to PEG6000) at concentrations of about 10 to 80%, and conducting incubation at 20 to 40° C., preferably at 30 to 37° C., for 1 to 10 minutes.

A monoclonal antibody-producing hybridoma can be screened for by, for example, a method wherein the hybridoma culture supernatant is added to a solid phase (e.g., microplate) having a protein antigen adsorbed thereto directly or along with a carrier, an anti-immunoglobulin antibody (when the cell used for cell fusion is a mouse cell, an anti-mouse immunoglobulin antibody is used) or protein A labeled with a radioactive substance, an enzyme or the like is then added, and the monoclonal antibody bound to the solid phase is detected, a method wherein the hybridoma culture supernatant is added to a solid phase having an anti-immunoglobulin antibody or protein A adsorbed thereto, a protein labeled with a radioactive substance, an enzyme or the like is added, and the monoclonal antibody bound to the solid phase is detected, and the like.

Selection of a monoclonal antibody can be conducted according to a method known per se or a method based thereon. Selection of a monoclonal antibody can normally be conducted using an animal cell culture medium supplemented with HAT (hypoxanthine, aminopterin, thymidine). As the medium for selection and breeding of a monoclonal antibody, any medium can be used, as long as the hybridoma can grow therein. As the medium, for example, an RPMI 1640 medium containing 1 to 20%, preferably 10 to 20%, fetal bovine serum, a GIT medium (Wako Pure Chemical Industries, Ltd.) containing 1 to 10% fetal bovine serum or a serum-free medium for hybridoma culture (SFM-101, Nissui Pharmaceutical Co., Ltd.) and the like can be used. Cultivation temperature is normally 20 to 40° C., preferably about 37° C. Cultivation time is normally 5 days to 3 weeks, preferably 1 week to 2 weeks. Cultivation can normally be conducted under 5% carbonic acid gas. The antibody titer of the hybridoma culture supernatant can be measured in the same manner as the above-desctibed measurement of the antibody titer in the antiserum.

The thus-obtained monoclonal antibody can be separated and purified according to a method known per se, for example, a method of immunoglobulin separation and purification [e.g., salting-out method, alcohol precipitation method, isoelectric point precipitation method, electrophoresis method, adsorption and desorption method using an ion exchanger (e.g., DEAE), ultracentrifugation method, gel filtration method, specific purification method wherein only the antibody is collected using an antigen-binding solid phase or an active adsorbent such as protein A or protein G, and its bond is dissociated to yield the antibody].

[Preparation of Polyclonal Antibody]

A polyclonal antibody against the transcriptional regulatory factor of the present invention can be produced according to a method known per se. For example, the polyclonal antibody can be produced by immunizing a mammal with an immune antigen (protein antigen) as is or a complex thereof with a carrier protein in the same manner as the above-described method of monoclonal antibody production, collecting the antibody-containing product of the present invention from the immunized animal, and separating and purifying the antibody.

Regarding the complex of an immune antigen and carrier protein used to immunize a mammal, any kind of carrier protein can be crosslinked at any mixing ratio of carrier and hapten, as long as an antibody against the carrier-crosslinked immunized hapten is efficiently produced; for example, a method wherein bovine serum albumin, bovine thyroglobulin, hemocyanin or the like is coupled at a ratio of about 0.1 to 20, preferably about 1 to 5, parts by weight per 1 part by weight of hapten, can be used.

For coupling of a hapten and a carrier, various condensing agents, for example, active ester reagents containing glutaraldehyde, carbodiimide, a maleimide active ester, a thiol group or a dithiopyridyl group, and the like can, be used.

The condensation product, as is or along with a carrier or a diluent, is administered to a mammal at a site permitting antibody production. To increase antibody productivity in this administration, complete Freund's adjuvant and incomplete Freund's adjuvant may be administered. The administration is normally conducted every 2 to 6 weeks, in a total of about 3 to 10 times.

A polyclonal antibody can be collected from blood, ascites fluid and the like, preferably blood, of a mammal immunized by the above-described method.

A measurement of the polyclonal antibody titer in the antiserum can be conducted in the same manner as the above-described measurement of antibody titer in the antiserum. Separation and purification of the polyclonal antibody can be conducted according to the same immunoglobulin separation and purification method as the above-described monoclonal antibody separation and purification.

Another preferred embodiment of the substance that suppresses the activity is the fructose responsive element of the present invention or a nucleic acid (preferably DNA) comprising the same. Here, the FRE of the present invention or a nucleic acid comprising the same is a decoy nucleotide for a DNA to which the transcriptional regulatory factor of the present invention binds. The nucleic acid can be produced according to the above-described method.

Because the nucleic acid is of low toxicity and is capable of suppressing the function of the transcriptional regulatory factor of the present invention (that is, transcription promoting activity on the SREBP-1c gene and the like) in the living body, it can be used as a prophylactic or therapeutic agent for a metabolic disorder involved in an abnormality of the expression of the SREBP-1c gene. The nucleic acid can be formulated and administered orally or non-orally to a mammal (for example, human, mouse, rat, rabbit, sheep, swine, bovine, horse, cat, dog, monkey, chimpanzee and the like) in the same manner as the inhibitor of the transcriptional regulatory factor of the present invention, which is obtained by the above-described screening method.

The nucleic acid can also be administered to the above-described mammal after insertion to an appropriate vector, for example, retrovirus vector, adenovirus vector, adenovirus-associated virus vector and the like.

The nucleic acid can be administered to the above-described mammal using a gene gun or a catheter like a hydrogel catheter, and can also be administered locally into the trachea as an inhalant after conversion to an aerosol.

The dosage of the prophylactic or therapeutic agent for a metabolic disorder which comprises the FRE of the present invention or a nucleic acid comprising the same, varies depending on target disease, subject of administration, route of administration and the like; in an adult patient suffered from hypertriglyceridemia (body weight 60 kg), for example, the dosage is about 0.1 to 100 mg, preferably about 1.0 to 50 mg, more preferably about 1.0 to 20 mg, per day, based on the nucleic acid, which is an active ingredient.

The substance that suppresses the production of the transcriptional regulatory factor of the present invention preferably includes a nucleic acid comprising a base sequence complementary to the base sequence encoding the transcriptional regulatory factor of the present invention, or a portion thereof. As the nucleic acid comprising a base sequence complementary to the base sequence that encodes the transcriptional regulatory factor of the present invention or a portion thereof (hereinafter also abbreviated as an “antisense nucleic acid of the present invention”), any nucleic acid can be mentioned, as long as it has a base sequence completely complementary, or substantially complementary, to the base sequence that encodes the transcriptional regulatory factor of the present invention (the NBP of the present invention or the RBMX analogous protein of the present invention) or a portion thereof, and acts to suppress the translation of the protein from the RNA that encodes the transcriptional regulatory factor of the present invention. As the “substantially complementary base sequence”, a base sequence capable of hybridizing to the base sequence that encodes the transcriptional regulatory factor of the present invention under the physiological conditions for the cell that expresses the protein, more specifically, a base sequence having a homology of about 70% or more, preferably about 80% or more, more preferably about 90% or more, and most preferably about 95% or more, to the complementary strand of the base sequence that encodes the transcriptional regulatory factor of the present invention or a partial base sequence thereof, and the like can be mentioned.

The antisense nucleic acid of the present invention can be designed and synthesized on the basis of information on the cloned or determined base sequence of the nucleic acid encoding the transcriptional regulatory factor of the present invention. Such a nucleic acid is capable of inhibiting the replication or expression of the gene that encodes the transcriptional regulatory factor of the present invention. Hence, the antisense nucleic acid of the present invention is capable of hybridizing to the RNA transcribed from the gene that encodes the transcriptional regulatory factor of the present invention, and capable of inhibiting the synthesis (processing) or function (translation into protein) of mRNA.

The target region of the antisense nucleic acid of the present invention is not subject to limitation as to the length thereof, as long as hybridization of the antisense nucleic acid results in the inhibition of the translation of the transcriptional regulatory factor of the present invention, and can be the entire sequence or a partial sequence of the RNA that encodes the transcriptional regulatory factor of the present invention; a partial sequence of about 15 bases for the shortest, and the entire sequence of the mRNA or initial transcription product for the longest, can be mentioned. Considering the ease of synthesis and the issue of antigenicity, an oligonucleotide comprising about 15 to about 30 bases is preferred, which, however, is not to be construed as limiting. Specifically, for example, the 5′-end hairpin loop, the 5′-end 6-base-pair repeat, the 5′-end untranslated region, the polypeptide translation initiation codon, the protein-coding region, the ORF translation initiation codon, the 3′-end untranslated region, the 3′-end palindrome region, and the 3′-end hairpin loop of the gene that encodes the transcriptional regulatory factor of the present invention can be selected as the target region, but any region within the gene can be selected as the target. For example, it is also preferable that the intron portion of the gene be the target region.

Furthermore, the antisense nucleic acid of the present invention may be one capable of not only hybridizing to the mRNA that encodes the transcriptional regulatory factor of the present invention or the initial transcription product thereof to inhibit the translation to the protein, but also binding to the gene that encodes the transcriptional regulatory factor of the present invention, which is double-stranded DNA, to form a triple strand (triplex) and inhibit the transcription of RNA.

As the antisense nucleic acid, a deoxyribonucleotide containing 2-deoxy-D-ribose, a ribonucleotide containing D-ribose, another type of nucleotide that is an N-glycoside of the purine or pyrimidine base, or another polymer having a non-nucleotide backbone (for example, commercially available protein nucleic acids and synthetic sequence specific nucleic acid polymers) or another polymer containing a special bond (however, this polymer comprises a nucleotide having a configuration that allows base pairing or base attachment as found in DNA and RNA) and the like can be mentioned. These may be double-stranded DNAs, single-stranded DNAS, double-stranded RNAs or single-stranded RNAs, or DNA:RNA hybrids, and may also non-modified polynucleotides (or non-modified oligonucleotides), those having a known modification added thereto, for example, those with a marker known in the relevant field, those with a cap, those methylated, those having 1 or more naturally occurring nucleotides substituted by analogues, those modified with an intramolecular nucleotide, for example, those having a non-charge bond (for example, methylphosphonate, phospho triester, phosphoramidate, carbamate and the like), those having a charged bond or a sulfur containing bond (for example, phosphorothioate, phosphorodithioate and the like), for example, those having a side chain group of a protein (nuclease, nuclease inhibitor, toxin, antibody, signal peptide, poly-L-lysine and the like), or a sugar (for example, monosaccharide and the like) and the like, those having an intercalating compound (for example, acridine, psoralen and the like), those containing a chelate compound (for example, metals, radioactive metals, boron, oxidizing metals and the like), or those containing an alkylating agent, those having a modified bond (for example, α anomer type nucleic acid and the like). Here, “nucleoside”, “nucleotide” and “nucleic acid” may include not only those containing the purine and pyrimidine bases, but also those containing another modified heterocyclic base. These modified products may contain a methylated purine and pyrimidine, an acylated purine and pyrimidine, or another heterocycle. The modified nucleotide and the modified nucleotide may also have their sugar portion modified by, for example, substitution of 1 or more hydroxyl groups by a halogen, an aliphatic group and the like, or conversion to a functional group such as an ether or an amine.

The antisense nucleic acid is RNA, DNA or a modified nucleic acid (RNA, DNA). As specific examples of the modified nucleic acid, sulfur derivatives and thiophosphate derivatives of nucleic acids, and those resistant to the decomposition like polynucleosideamide or oligonucleosideamide can be mentioned, which, however, are not to be construed as limiting. The antisense nucleic acid of the present invention can preferably be designed to accomplish one of the following purposes: to make the antisense nucleic acid more stable in the cell, to increase the cell permeability of the antisense nucleic acid, to increase the affinity for the desired sense strand, and to reduce the toxicity, if any, of the antisense nucleic acid. Many such modifications are known in the relevant field, and are disclosed in, for example, J. Kawakami et al., Pharm Tech Japan, Vol. 8, pp. 247, 1992, Vol. 8, pp. 395, 1992; S. T. Crooke et al. ed., Antisense Research and Applications, CRC Press, 1993 and the like.

The antisense nucleic acid may be altered, and may contain an modified sugar, base or bond, and can be supplied in a special form like liposome or microspheres, can be applied for gene therapy, and can be given in an adduct form. As such an adduct form used, a polycation like polylysine, which acts to neutralize the charge of the phosphate backbone, and a hydrophobic compound like a lipid that enhances the interaction with cell membrane or increases nucleic acid uptake (for example, phospholipid, cholesterol and the like) can be mentioned. As lipids preferred for addition, cholesterol and derivatives thereof (for example, cholesterylchloroformate, cholic acid and the like) can be mentioned. These can be attached to the 3′ end or the 5′ end of nucleic acid, and can be attached via a base, a sugar or an intramolecular nucleoside bond. As other groups, a capping group specifically arranged at the 3′ end or 5′ end of nucleic acid to prevent degradation by a nuclease such as exonuclease or RNase can be mentioned. As such a capping group, hydroxyl group protecting groups known in the relevant field, including glycols such as polyethylene glycol and tetraethylene glycol can be mentioned, which, however, are not to be construed as limiting.

A ribozyme capable of specifically cleaving the mRNA or the initial transcription product that encodes the transcriptional regulatory factor of the present invention within the coding region (including the intron portion in the case of the initial transcription product) can also be encompassed in the antisense nucleic acid of the present invention. “Ribozyme” refers to RNA possessing an enzyme activity to cleave a nucleic acid, and is herein understood to be used as a concept encompassing DNA, as long as sequence-specific nucleic acid cleavage activity is possessed, since it has recently been found that oligo DNA having the base sequence of the enzyme activity portion also possesses nucleic acid cleavage activity. One of the most versatile ribozymes is self-splicing RNA found in infectious RNAs such as viroid and virusoid, and the hammerhead type, the hairpin type and the like are known. The hammerhead type exhibits enzyme activity with about 40 bases in length, and it is possible to specifically cleave the target mRNA by making several bases at both ends adjoining to the hammerhead structure portion (about 10 bases in total) to be a sequence complementary to the desired cleavage site of the mRNA. Because this type of ribozymes has RNA only as the substrate, it offers an additional advantage of non-attack of genomic DNA. Provided that the mRNA encoding the transcriptional regulatory factor of the present invention takes a double-stranded structure by itself, the target sequence can be made single-stranded, using a hybrid ribozyme prepared by joining an RNA motif derived from a viral nucleic acid that can specifically bind to RNA helicase [Proc. Natl. Acad. Sci. USA, 98(10): 5572-5577 (2001)]. Furthermore, when the ribozyme is used in the form of an expression vector comprising the DNA that encodes it, the ribozyme may be a hybrid ribozyme prepared by further joining a sequence modified from the tRNA to promote the migration of the transcription product to cytoplasm [Nucleic Acids Res., 29(13): 2780-2788 (2001)].

A double-stranded oligo RNA complementary to a partial sequence (including the intron portion in the case of the initial transcription product) within the coding region of the mRNA or the initial transcription product that encodes the transcriptional regulatory factor of the present invention (small interfering RNA; siRNA) can also be encompassed in the antisense nucleic acid of the present invention. RNA interference (RNAi), a phenomenon in which introducing short double-stranded RNA in a cell leads to the decomposition of mRNA complementary to the RNA, has been known to occur in nematodes, insects, plants and the like, and since this phenomenon has recently been found to occur in mammalian cells as well [Nature, 411(6836): 494-498 (2001)], it is attracting attention for technology to replace ribozymes.

The antisense oligonucleotide and ribozyme of the present invention can be prepared by determining the target region of the mRNA or initial transcription product on the basis of information on the cDNA sequence or genomic DNA sequence that encodes the transcriptional regulatory factor of the present invention, and synthesizing a sequence complementary thereto using a commercially available DNA/RNA synthesizer (Applied Biosystems, Beckman Instruments, and the like). siRNA can be prepared by synthesizing each of a sense strand and an antisense strand using a DNA/RNA synthesizer, denaturing the strands in an appropriate annealing buffer at, for example, about 90 to about 95° C. for about 1 minute, and then annealing the strands at about 30 to about 70° C. for about 1 to about 8 hours. It is also possible to prepare a longer double-stranded polynucleotide by synthesizing complementary oligonucleotide strands in alternative overlaps, annealing the strands, and then ligating the strands using ligase.

When the aforementioned “substance that suppresses the production” is the antisense nucleic acid of the present invention, the antisense nucleic acid can be administered to a mammal as a prophylactic or therapeutic agent for a metabolic disorder after insertion into an appropriate vector, for example, retrovirus vector, adenovirus vector, and adenovirus associated virus vector.

The antisense nucleic acid can be administered using a gene gun or a catheter like a hydrogel catheter, and can also be administered locally into the trachea as an inhalant after conversion to an aerosol.

Furthermore, the antisense nucleic acid of the present invention can also be used as a diagnostic oligonucleotide probe to examine the presence and the expression manner of a nucleic acid that encodes the transcriptional regulatory factor of the present invention in a tissue or cell.

The present invention also provides a protein or peptide comprising an amino acid sequence having one or more amino acids substituted, deleted, inserted, or added in the transcriptional regulatory factor of the present invention or a partial peptide thereof, which is capable of binding to the fructose responsive element of the present invention but does not activate a promoter comprising the FRE sequence. “Does not activate a promoter” means the inability to promote the transcriptional activation of a gene under the control of the promoter. The protein or peptide may be any one, as long as it possesses the above-described characteristics; for example, a mutant protein or peptide lacking the transcription promoting activity due to a substitution, deletion, insertion or addition of an amino acid in the transcription regulatory (activation) domain of the NBP or RBMX analogous protein of the present invention can be mentioned.

Because the above-described mutant protein or peptide is capable of inhibiting the binding of the normal transcriptional regulatory factor of the present invention to the FRE sequence and the transcription activation on an SREBP-1c gene by binding to an SREBP-1c promoter comprising the FRE sequence of the present invention, it is useful in the prophylaxis and treatment of a metabolic disorder associated with an abnormally increased expression of the SREBP-1c gene, especially glucose or lipid metabolic disorder (e.g., hypertriglyceridemia, hyper-LDL-cholesteremia, hypo-HDL-cholesterolemia, obesity, abnormality of glucose tolerance, fasting blood glucose disorder, hyperinsulinemia, hypertension, albuminuria, and the like). Accordingly, the present invention provides a prophylactic or therapeutic agent for a metabolic disorder, especially for a glucose or lipid metabolic disorder, which contains the mutant protein or peptide.

Although the above-described prophylactic or therapeutic agent for a metabolic disorder, which contains the mutant protein or peptide, may be the mutant protein or peptide as is, it is preferably a pharmaceutical composition prepared by mixing it with a pharmacologically acceptable carrier. Here, as a pharmacologically acceptable carrier, the same as “the inhibitor of the transcriptional regulatory factor of the present invention” as obtained by the aforementioned screening method can be mentioned.

The pharmaceutical composition can be produced in the same manner as the aforementioned “inhibitor of the transcriptional regulatory factor of the present invention”.

Because the preparation thus obtained is safe and of low toxicity, it can be administered orally or non-orally to, for example, a mammal (for example, human, mouse, rat, rabbit, sheep, swine, bovine, horse, cat, dog, monkey, chimpanzee and the like).

The dosage of the prophylactic or therapeutic agent for metabolic disorder in accordance with the present invention varies depending on target disease, subject of administration, route of administration and the like; in an adult patient suffered from hypertriglyceridemia (body weight 60 kg), for example, the dosage is about 0.1 to 100 mg, preferably about 1.0to 50 mg, more preferably about 1.0 to 20 mg, per day, based on the mutant protein or peptide, which is an active ingredient.

The present invention also relates to a diagnostic reagent for metabolic disorder, especially glucose or lipid metabolic disorder (e.g., hypertriglyceridemia, hyper-LDL-cholesteremia, hypo-HDL-cholesterolemia, obesity, abnormality of glucose tolerance, fasting blood glucose disorder, hyperinsulinemia, hypertension, albuminuria, and the like), which comprises the above-described nucleic acid having a base sequence that encodes the transcriptional regulatory factor of the present invention, or a portion thereof. For example, because it is possible to detect an abnormality (genetic abnormality) in the DNA or mRNA that encodes the transcriptional regulatory factor of the present invention in a mammal (for example, human, rat, mouse, guinea pig, rabbit, sheep, swine, bovine, horse, cat, dog, monkey, chimpanzee and the like) using a nucleic acid having a base sequence encoding the transcriptional regulatory factor of the present invention as a probe, the nucleic acid is useful as, for example, a genetic diagnostic reagent for damage, mutation or decreased expression of the DNA or mRNA, increased expression or overexpression of the DNA or mRNA, and the like.

The above-described genetic diagnosis can be performed by, for example, a method known per se, such as Northern hybridization and the PCR-SSCP method (Genomics, Vol. 5, pp. 874 to 879 (1989), Proceedings of the National Academy of Sciences of the United States of America, Vol. 86, pp. 2766 to 2770 (1989)) and the like.

For example, if bverexpression is detected by Northern hybridization, or if a DNA mutation is detected by the PCR-SSCP method, the test animal could be diagnosed as being likely to have glucose or lipid metabolic disorder such as hypertriglyceridemia.

The present invention also relates to a diagnostic reagent for metabolic disorder, especially glucose or lipid metabolic disorder (e.g., hypertriglyceridemia, hyper-LDL-cholesteremia, hypo-HDL-cholesterolemia, obesity, abnormality of glucose tolerance, fasting blood glucose disorder, hyperinsulinemia, hypertension, albuminuria, and the like), which contains the above-described antibody of the present invention.

Accordingly, the present invention provides:
(i) a method of diagnosing metabolic disorder, especially glucose or lipid metabolic disorder, which comprises competitively reacting the antibody of the present invention, a test solution, and the transcriptional regulatory factor of the present invention (including the partial peptide) being labeled, and determining the ratio of the labeled transcriptional regulatory factor of the present invention bound to the antibody, to quantity the transcriptional regulatory factor of the present invention or a salt thereof in the test solution, and
(ii) a method of diagnosing metabolic disorder, especially glucose or lipid metabolic disorder, which comprises reacting a test solution, an antibody of the present invention insolubilized on a carrier, and another antibody of the present invention being labeled, simultaneously or serially, and then measuring the activity of the labeling agent on the insolubilizing carrier to quantity the transcriptional regulatory factor of the present invention or a salt thereof in the test solution.

In the quantitation of (ii) above, if one of the two antibodies is an antibody that recognizes an N-terminal portion of the transcriptional regulatory factor of the present invention, the other antibody is desirably an antibody that recognizes another portion, for example, a C-terminal portion, of the transcriptional regulatory factor of the present invention.

In addition to the quantitation of the transcriptional regulatory factor of the present invention using a monoclonal antibody against the protein, detection by tissue staining and the like can also be conducted. For these purposes, the antibody molecule itself may be used, and the F(ab′)₂, Fab′ or Fab fraction of the antibody molecule may also be used. Furthermore, single chain antibody linking variable regions of heavy chain and light chain (scFV) can be used.

The quantitation of the transcriptional regulatory factor of the present invention or a salt thereof using the antibody of the present invention is not subject to limitation, and any method of measurement can be used, as long as it is a measurement method wherein the amount of antibody, antigen or antibody-antigen complex; corresponding to the amount of antigen in the test solution is detected by a chemical or physical means and is calculated on the basis of a standard curve generated using standard solutions containing known amounts of antigen. For example, nephelometry, the competitive method, the immunometric method and the sandwich method are preferably used; it is particularly preferable, in terms of sensitivity and specificity, to use the sandwich method described below.

As examples of the labeling agent used for the measurement method using a labeled substance, a radioisotope, an enzyme, a fluorescent substance, a luminescent substance and the like can be used. As examples of the radioisotope, [¹²⁵I], [¹³¹I], [³H], [¹⁴C] and the like can be used. As the above-described enzyme, those that are stable and high in specific activity are preferred; for example, β-galactosidase, β-glucosidase, alkaline phosphatase, peroxidase, malate dehydrogenase and the like can be used. As examples of the fluorescent substance, fluorescamine, fluorescein isothiocyanate and the like can be used. As examples of the luminescent substance, luminol, luminol derivative, luciferin, lucigenin and the like can be used. Furthermore, a biotin-(strepto)avidin system can also be used for binding of an antibody or an antigen and a labeling agent.

In insolubilizing the antigen or antibody, physical adsorption may be used, and a method based on a chemical bond conventionally used to insolubilize or immobilize a protein or the like, may also be used. As the carrier, insoluble polysaccharides such as agarose, dextran and cellulose, synthetic resins such as polystyrene, polyacrylamide and silicone, glass and the like can be mentioned.

In the sandwich method, the amount of the transcriptional regulatory factor of the present invention or a salt thereof in a test solution can be quantified by reacting the test solution to a monoclonal antibody of the present invention insolubilized (primary reaction) and further reacting to another monoclonal antibody of the present invention labeled (secondary reaction), and thereafter measuring the activity of the labeling agent on the insolubilizing carrier. The primary reaction and the secondary reaction may be conducted in the reverse order, and may be conducted simultaneously or after a time lag. The labeling agent and the method of insolubilization can be based on those described above. Also, in the immunoassay by the sandwich method, the antibody used as the antibody for a solid phase or the antibody for labeling needs not always be one kind; a mixture of two kinds or more of antibodies may be used for the purposes of measurement sensitivity improvement and the like.

In the above-described measurement of the transcriptional regulatory factor of the present invention or a salt thereof by the sandwich method, as the monoclonal antibodies of the present invention used in the primary reaction and the secondary reaction, antibodies having mutually different sites for binding of the transcriptional regulatory factor of the present invention are preferably used. Accordingly, as the antibodies used for the primary reaction and the secondary reaction, provided that the antibody used for the secondary reaction recognizes a C-terminal portion of the transcriptional regulatory factor of the present invention, for example, the antibody used for the primary reaction is preferably an antibody that recognizes a site other than the C-terminal portion, for example, an N-terminal portion are used.

The monoclonal antibody of the present invention can be used for a measurement system other than the sandwich method, for example, the competitive method, the immunometric method or nephelometry and the like.

In the competitive method, the antigen and the labeled antigen in the test solution are competitively reacted with the antibody, after which the unreacted labeled antigen (F) and the antibody-bound labeled antigen (B) are separated (B/F separation), the amount labeled of either B or F is measured, and the amount of antigen in the test solution is quantified. For this reaction method, the liquid phase method, wherein a soluble antibody is used as the antibody and B/F separation is conducted using polyethylene glycol, a second antibody against the above-described antibody, and the like, and the solid phase immobilization method, wherein a solid-phase-immobilized antibody is used as the first antibody or the first antibody used is a soluble one and a solid-phase-immobilized antibody is used as the second antibody, can be used.

In the immunometric method, the antigen and the solid phase-immobilized antigen in the test solution are competitively reacted to a given amount of labeled antibody, after which the solid phase and the liquid phase are separated, or the antigen in the test solution and an excess amount of labeled antibody are reacted, a solid-phase-immobilized antigen is then added to bind the unreacted labeled antibody to the solid phase, after which the solid phase and the liquid phase are separated. Next, the amount labeled in either phase is measured to quantify the antigen in the test solution.

Also, in nephelometry, the amount of insoluble precipitate resulting from an antigen-antibody reaction in the gel or in the solution is measured. Even when the amount of antigen in the test solution is small and only a small amount of precipitate is obtained, laser nephelometry, which utilizes laser scattering, and the like are preferably used.

In applying these individual immunoassays to the quantitation method of the present invention, it is unnecessary to set special conditions, procedures and the like. Making ordinary technical considerations for those skilled in the art to the ordinary conditions and procedures in each method, a measurement system for the transcriptional regulatory factor of the present invention can be constructed. For details of these general technical means, compendia, books and the like can be referred to.

For example, edited by Hiroshi Irie, “Rajioimunoassei” (Kodansha, published in 1974), edited by Hiroshi Irie, “Zoku Rajioimunoassei” (Kodansha, published in 1979), edited by Eiji Ishikawa et al., “Kouso Meneki Sokuteihou” (Igaku-Shoin, published in 1978), edited by Eiji Ishikawa et al., “Kouso Meneki Sokuteihou” (2nd edition) (Igaku-Shoin, published in 1982), edited by Eiji Ishikawa, “Kouso Meneki Sokuteihou” (3rd edition) (Igaku-Shoin, published in 1987), “Methods in ENZYMOLOGY”, Vol. 70 (Immunochemical Techniques (Part A)), ibidem, Vol. 73 (Immunochemical Techniques (Part B)), ibidem, Vol. 74 (Immunochemical Techniques (Part C)), ibidem, Vol. 84 (Immunochemical Techniques (Part D: Selected Immunoassays)), ibidem, Vol. 92 (Immunochemical Techniques (Part E: Monoclonal Antibodies and General Immunoassay Methods)), ibidem, Vol. 121 (Immunochemical Techniques (Part I: Hybridoma Technology and Monoclonal Antibodies)) (all published by Academic Press) and the like can be referred to.

Using the antibody of the present invention as described above, the transcriptional regulatory factor of the present invention or a salt thereof can be quantified at high sensitivity.

In the above-described quantitation method using the antibody of the present invention, the concentration of the transcriptional regulatory factor of the present invention or a salt thereof in a biological sample (e.g., kidney cell, pancreatic cell and the like) from a test animal as analyte is quantified; if overexpression expression of the factor is detected, the test animal could be diagnosed as being likely to suffer from glucose or lipid metabolic disorder such as hypertriglyceridemia.

The present invention also provides a non-human transgenic animal incorporating a gene under the control of a promoter comprising the fructose responsive element (FRE) of the present invention. As examples of the “promoter comprising the FRE of the present invention”, a promoter prepared by joining the FRE of the present invention to the appropriate position of the same animal cell promoter as described with respect to the above-described screening method of the present invention, using a gene engineering technique, and the like can be mentioned. Alternatively, an SREBP-1c promoter comprising the FRE of the present invention as is can also be used as a “a promoter comprising the FRE of the present invention”.

As examples of the gene under the control of the promoter, the same reporter gene as described with respect to the above-described screening method of the present invention can preferably be mentioned, and an SREBP-1c gene comprising the FRE of the present invention in the promoter region thereof as is can also be used as a “gene under the control of a promoter comprising the FRE of the present invention”.

Here, the “transgenic animal” means that a gene under the control of a promoter comprising the FRE of the present invention is permanently present in an expressible state in host animal cells; although the gene may be incorporated into the host chromosome or stably present as an extrachromosomal gene, the gene is preferably retained as incorporated on the host chromosome.

A non-human transgenic animal transferred with a gene under control of a promoter comprising the fructose responsive element of the present invention (hereinafter abbreviated as a “FRE-Tg animal of the present invention”) is produced by introducing the desired gene to a fertilized ovum, an unfertilized ovum, a sperm, or a precursor cell thereof (primordial germ cell, oogonium, oocyte, ovum, gonocyte, spermatocyte, spermatid and the like) or the like of non-human animal, preferably in the early stage of embryogenesis in fertilized ovum (more preferably, at or prior to the 8-cell stage), by the gene transfer method such as calcium phosphate method, the electric pulse (electroporation) method, the lipofection method, the aggregation method, the microinjection method, the particle gun method, the DEAE-dextran method and the like. Also, it is possible to introduce the desired gene to a somatic cell, a tissue, an organ or the like of non-human mammal by the gene transfer method, and utilize it for cell culture, tissue culture and the like; furthermore, it is also possible to produce the transgenic animal by fusing these cells with the above-described embryonic (or germ) cell by a method of cell fusion known per se. Alternatively, a transgenic animal can also be obtained, in the same manner as producing a knockout animal, by introducing the gene of interest into embryonic stem cells (ES cells) of the non-human mammal using the above-described gene transfer method, selecting clones stably incorporating the gene, injecting the ES cells into a blastocyst or aggregating an ES cell mass with 8-cell embryos to produce chimeric mice, and selecting one in which the transferred DNA has been introduced to germ line.

A part of the living body of the transgenic animal produced in this manner (for example, 1) a cell, tissue, organ or the like stably retaining a transferred gene; 2) ones obtained by culturing and, if necessary, subculturing a cell or tissue derived therefrom, and the like) can be used for the same purpose as “the FRE-Tg animal of the present invention” as “a part of the living body of the FRE-Tg animal of the present invention.” As examples of the part of the living body of the FRE-Tg animal of the present invention, organs such as the liver, heart, kidney, adrenal gland, blood vessels, gastrointestinal tract and brain, and tissues and cells such as tissue sections and cells derived from these organs, and the like can be preferably mentioned.

The “non-human mammal” that can be used as the subject of the present invention is not subject to limitation, as long as it is a non-human mammal for which a transgenic system has been established, and includes, for example, bovine, swine, sheep, goat, rabbit, dog, cat, guinea pig, hamster, rat, mouse and the like. Preferably, the non-human mammal is mouse, rat, rabbit, dog, cat, guinea pig, hamster or the like. Particularly preferred from the viewpoint of preparation of a pathologic animal model are rodents, which have relatively short ontogenesis and biological cycles, and which permit easy propagation, particularly the mouse (for example, C57BL/6 strain, DBA/2 strain and the like as pure strains, B6C3F₁ strain, BDF₁ strain, B6D2F₁ strain, BALB/c strain, ICR strain and the like as cross strains) or the rat (for example, Wistar, SD and the like).

Also, in addition to mammals, birds such as chicken can be used for the same purpose as that of a “non-human mammal” that is the subject of the present invention.

Although the structural gene in the transferred gene is preferably in an intron-free form (that is, a cDNA), an intron-comprising form (that is, a genomic DNA) can also be used preferably in another embodiment because the 5′ and 3′ terminal sequences are common to almost all eukaryotic genes.

As the expression vector carrying a transgene, an Escherichia coli-derived plasmid, a Bacillus subtilis-derived plasmid, a yeast-derived plasmid, a bacteriophage such as λ phage, a retrovirus such as Moloney leukemia virus, an animal virus such as vaccinia virus or baculovirus, and the like can be used. Particularly preferably used are an Escherichia coli-derived plasmid; a Bacillus subtilis-derived plasmid or a yeast-derived plasmid and the like. Especially, an Escherichia coil-derived plasmid is preferred.

The transqene preferably has a sequence that terminates the transcription of the desired mRNA in the transgenic animal (also referred to as polyadenylation (poly A) signal or terminator) at downstream thereof; for example, the terminator sequence derived from virus genes and derived from genes of various mammals or birds can be used to achieve efficient expression of the transgene, and the SV40 terminator of simian virus and the like can be used preferably. Besides, for the purpose of expressing the desired gene at a higher level, the splicing signal and an enhancer region of each gene, and a portion of the intron of a eukaryotic gene can also be joined upstream of the 5′ of the promoter region, between the promoter region and the translation region (5′ UTR) or downstream of the 3′ of the translation region (3′ UTR) depending on the purpose.

Also, when a transgenic animal is prepared using an ES cell, the above-described vector preferably further comprises a selection marker gene (e.g., drug resistance genes such as the neomycin resistance gene and the hygromycin resistance gene) to select a clone having the transgene incorporated stably therein. Furthermore, when it is intended to incorporate the transgene in a particular site of a host chromosome by homologous recombination (that is, preparation of a knock-in animal), the above-described vector preferably further comprises the herpes simplex virus-derived thymidine kinase gene or the diphtheria toxin gene, as the negative selection marker gene, outside a DNA sequence homologous to the target site, in order to eliminate random insertions. These modes of embodiment are described in detail below.

The above-described promoter, structural gene DNA, terminator and the like can be inserted to the above-described vector in the correct arrangement, that is, in an arrangement that enables the expression of the transgene in the transgenic animal, by an ordinary gene engineering technique using an appropriate restriction enzyme, DNA ligase and the like.

In a preferred embodiment, the expression vector comprising a gene under the control of a promoter comprising the FRE of the present invention, obtained as described above, is transferred to an early embryo of the subject non-human mammal by the microinjection method.

An early embryo of the subject non-human mammal can be obtained by collecting an internally fertilized egg obtained by mating a female and a male of the same species of non-human mammal, or by externally fertilizing an ovum and sperm collected from a female and a male, respectively, of the same species of non-human mammal.

The age, rearing conditions and the like for the non-human mammal used vary depending on the animal species; when using the mouse (preferably an inbred mouse such as C57BL/6J (B6), F₁ of B6 and another inbred strain, and the like), for example, it is preferable that the female be at about 4 to about 6 weeks of age, and the male be at about 2 to about 8 months or so of age, and is also preferable that they be reared under about 12-hour bright phase conditions (for example, 7:00-19:00) for about 1 week.

Although internal fertilization may be by spontaneous mating, a method wherein for the purpose of regulating the sexual cycle and obtaining a large number of early embryos from one animal, gonadotropin is administered to a female non-human mammal to induce superovulation, and thereafter the female is mated with a male non-human mammal, is preferred. As examples of the method of inducing ovulation in a female non-human mammal, a method wherein follicle-stimulating hormone (pregnant mare's serum gonadotropin, generally abbreviated as PMSG) is first administered, then luteinizing hormone (human chorionic gonadotropin, generally abbreviated as hCG) is administered, by, for example, intraperitoneal injection and the like, is preferred; the preferable hormone dosage and administration interval respectively vary depending on the species of non-human mammal. For example, when the non-human mammal is the mouse (preferably an inbred mouse such as C57BL/6J (B6), F₁ of B6 and another inbred strain, and the like), a method wherein a fertilized egg is obtained by administering luteinizing hormone at about 48 hours after administration of follicle-stimulating hormone, and thereafter immediately mating the female with a male mouse, is usually preferred; the dosage of follicle-stimulating hormone is about 20 to about 50 IU/animal, preferably about 30 IU/animal, and the dosage of luteinizing hormone is about 0 to about 10 IU/animal, preferably about 5 IU/animal.

After a given time has elapsed, the peritoneum of each female non-human mammal confirmed by vaginal plug testing and the like to have copulated was incised, and fertilized eggs are taken out from the oviduct, washed in a medium for embryo culture (e.g., M16 medium, modified Whitten medium, BWW medium, M2 medium, WM-HEPES medium, BWW-HEPES medium and the like) to remove cumulus cells, and cultured by the droplet culture method and the like in the presence of 5% carbonic acid gas/95% atmosphere until the time of DNA microinjection. When microinjection is not immediately conducted, it is also possible to preserve the collected fertilized eggs under freezing by the slow method or the ultrarapid method and the like.

On the other hand, in the case of external fertilization, follicle-stimulating hormone and luteinizing hormone are administered to a female non-human mammal for egg collection (the same as in the case of internal fertilization is preferably used) in the same manner as above to induce ovulation, after which eggs are collected and cultured in a medium for fertilization (e.g., TYH medium) until the time of external fertilization by the droplet culture method and the like in the presence of 5% carbonic acid gas/95% atmosphere. On the other hand, the tail of the epididymis is taken out from the same species of male non-human mammal (the same as in the case of internal fertilization is preferably used), and a sperm mass is collected and pre-cultured in a medium for fertilization. After completion of the pre-culture, the sperm is added to an egg-containing medium for fertilization; after cultivation by the droplet culture method and the like in the presence of 5% carbonic acid gas/95% atmosphere, fertilized eggs having two pronuclei are selected under a microscope. When DNA microinjection is not immediately conducted, it is also possible to preserve the collected fertilized eggs under freezing by the slow method or the ultrarapid method and the like.

DNA microinjection to a fertilized egg can be performed using a publicly known apparatus such as a micromanipulator according to a conventional method. Briefly speaking, the fertilized egg placed in a droplet of a medium for embryo culture is aspirated and immobilized using a holding pipette, and a DNA solution is injected directly to the male or female pronucleus, preferably into the male pronucleus, using an injection pipette. The transferred DNA used is preferably one that has been highly purified by CsCl density gradient ultracentrifugation and the like. Also, the transferred DNA is preferably linearized by cutting the vector portion thereof using a restriction enzyme.

After the DNA transfer, the fertilized egg is cultured in a medium for embryo culture by the droplet culture method and the like in the presence of 5% carbonic acid gas/95% atmosphere until the 1-cell stage-blastocyst stage, after which it is transplanted into the oviduct or uterus of a female non-human mammal for embryo reception rendered to be pseudopregnant. The female non-human mammal for embryo reception may be any female, as long as it is of the same species as the animal from which the early embryo to be transplanted is derived; for example, when a mouse early embryo is transplanted, a female ICR strain mouse (preferably about 8 to about 10 weeks of age) and the like are preferably used. As an example of the method of rendering the female non-human mammal for embryo reception to be in a pseudopregnant state, a method wherein the female is mated with the same species of vasectomized (vasoligated) male non-human mammal (for example, in the case of a mouse, a male ICR strain mouse (preferably about 2 months or more of age)), and selecting one confirmed as having a vaginal plug, is known.

The female for embryo reception used may be a spontaneously ovulating female, or a female having fertility induced by administering luteinizing hormone-releasing hormone (generally abbreviated as LHRH) or an analog thereof prior to mating with a vasectomized (vasoligated) male. As examples of the LHRH analog, [3,5-DiI-Tyr⁵]-LH-RH, [Gln⁸]-LH-RH, [D-Ala⁶]-LH-RH, [des-Gly¹⁰]-LH-RH, [D-His (Bzl)⁶] -LH-RH, Ethylamides thereof and the like can be mentioned. The dosage of LHRH or an analog thereof, and the timing of mating with a male non-human mammal after administration thereof vary depending on the species of non-human mammal. For example, when the non-human mammal is the mouse (preferably an ICR strain mouse and the like), it is usually preferable that the female mouse be mated with a male mouse at about 4 days after LHRH or an analog thereof is administered; the dosage of LHRH or an analog thereof is usually about 10 to 60 μg/animal, preferably about 40 μg/animal.

Usually, when the early embryo to be transplanted is in the morula stage or after, it is transplanted to the uterus of a female for embryo reception; when the early embryo is in an earlier stage (for example, 1-cell stage to 8-cell stage embryo), it is transplanted to the oviduct. As the female for embryo reception, one which is older than a given number of days from pseudopregnancy, depending on the developmental stage of the transplanted embryo, is appropriately used. For example, in the case of the mouse, a female mouse at about 0.5 days after pseudopregnancy is preferred for transplantation of a 2-cell stage embryo, and a female mouse at about 2.5 days after pseudopregnancy is preferred for transplantation of a blastocystic embryo. After the female for embryo reception is anesthetized (preferably Avertin and the like are used), an incision is made, the ovary is drawn out, early embryo (about 5 to about 10 cells) in suspension in a medium for embryo culture are injected to the peritoneal opening of the oviduct or the vicinity of the oviduct junction of the uterine horn using a pipette for embryo transplantation.

If the transplanted embryo successfully implants and the embryo recipient female becomes pregnant, non-human mammal pups are obtained by spontaneous delivery or caesarian section. Embryo recipient females that delivered spontaneously are allowed to continue suckling; if the pups are delivered by caesarian section, the pups can be suckled by a separately provided female for suckling (for example, in the case of the mouse, a female mouse with usual mating and delivery (preferably female ICR strain mouse and the like)).

Referring to the transfer of a gene in the fertilized egg cell stage, it is assured that the transferred DNA is present in all germ line cells and somatic cells of the subject non-human mammal. Whether or not the transferred DNA is incorporated in the chromosome DNA can be determined by, for example, screening chromosome DNAs separated and extracted from the tails of offspring pups, by Southern hybridization or PCR method. The presence of the transferred DNA in the germ line cells of non-human mammal pups (F₀) obtained as described above means that a gene under the control of a promoter comprising the FRE of the present invention is present in all of the germ line cells and somatic cells of all progeny (F₁) animals.

Usually, the Fo animals are obtained as heterozygotes having the transferred DNA in only one of the homologous chromosomes. Also, transferred DNA is randomly inserted onto different chromosomes in individual F₀ animals unless produced by homologous recombination. To obtain a homozygote having the transferred DNA on both homologous chromosomes, an F₀ animal and a non-transgenic animal are crossed to prepare F₁ animals, and siblings of a heterozygote having the transferred DNA in only one of the homologous chromosomes are crossed. Provided that the transferred DNA has been incorporated in only one gene locus, one-fourth of the obtained F₂ animals would be homozygotes.

In a preferred embodiment, the expression vector comprising a gene under the control of a promoter comprising the FRE of the present invention is transferred to an embryonic stem cell (ES cell) of the subject non-human mammal by a publicly known method of gene transfer such as the electroporation method.

An ES cell refers to a cell which is derived from the inner cell mass (ICM) of a fertilized egg in the blastocyst stage, and which can be cultured and maintained while retaining an undifferentiated state in vitro. ICM cells are cells that will form the embryo itself and are also stem cells on which all tissues, including germ cells, are based. The ES cell may be of an already established cell line, and may also be newly established in accordance with the method of Evans and Kaufman (Nature, Vol. 292, p. 154, 1981). In the case of a mouse ES cell, for example, an ES cell derived from a 129 strain mouse, is currently generally used; however, since its immunological background is unclear, an ES cell established from C57BL/6 mice or BDF₁ mice (F₁ of C57BL/6 and DBA/2), which has been developed by improving the low number of eggs collectable from C57BL/6 by crossing with DBA/2, and the like, for example, can also be used favorably for the purpose of obtaining an ES cell of a pure strain having an immunologically clear genetic background, in place of the ES cell derived from a 129 strain mouse, and for other purposes. In addition to being advantageous in that the number of collectable eggs is large and the eggs are tough, BDF₁ mice have a background association with C57BL/6 mice; therefore, ES cells derived therefrom are advantageously usable in that the genetic background thereof can be replaced with that of C57BL/6 mice by being back-crossed with C57BL/6 mice when a disease model mouse is prepared.

Preparation of an ES cell can, for example, be conducted as described below. A blastocystic embryo is collected from the uterus of a mated female non-human mammal [when using a mouse (preferably an inbred mouse such as C57BL/6J (B6), F₁ of B6 and another inbred strain, and the like), for example, a female mouse at about 8 to about 10 weeks of age (about 3.5 days of gestation) mated with a male mouse at about 2 months or more of age is preferably used] (or it is also possible to collect an early embryo in the morula stage or before from the oviduct, and thereafter culture it in a medium for embryo culture in the same manner as above until the blastocyst stage), and cultured on a layer of appropriate feeder cells (for example, in the case of the mouse, a primary fibroblast prepared from a mouse fetus, a known STO fibroblast line and the like), whereby some cells of the blastocyst aggregate to form an ICM which will differentiate into an embryo. This inner cell mass is trypsinized to dissociate the single cells, and dissociation and passage are repeated while maintaining an appropriate cell density and conducting medium exchanges, whereby an ES cell is obtained.

Although the ES cell may be of either sex, a male ES cell is usually more convenient that sex identification be conducted as soon as possible for preparation of a germ line chimera. Also, it is desirable, also for saving labor for painstaking cultivation. As an example of the ES cell sex identification method, a method wherein the gene in the sex determination region on the Y chromosome is amplified and detected by the PCR method can be mentioned. Using this method, the number of ES cells can be reduced to about 1 colony (about 50 cells), in contrast to the conventional practice that requires a cell number of about 10⁶ cells for karyotype analysis, so that primary selection of ES cells in the initial stage of cultivation can be conducted by sex identification, which in turn makes it possible to significantly save labor in the initial stage of cultivation because early selection of male cells has been made possible.

Also, secondary selection can be conducted by, for example, confirmation of the number of the chromosome by the G-banding method, and the like. Although the number of the chromosome in the ES cells obtained is desirably 100% of the normal number, it is desirable that if the 100% level is difficult to achieve for the reasons of physical operation and the like at the time of cell line establishment, transfer into the ES cells be followed by re-cloning into normal cells (for example, cells having the number of the chromosome of 2n=40 in the case of the mouse).

The ES cell line thus obtained need to be carefully subcultured to maintain its property of undifferentiated stem cell property. For example, a method wherein the embryonic stem cell line is cultured orn appropriate feeder cells like the STO fibroblast, in the presence of LIF (1 to 10,000 U/ml) which is known as an inhibitor of differentiation, in a carbonic acid gas incubator (preferably 5% carbonic acid gas/95% air, or 5% oxygen/5%-carbonic acid gas/90% air) at about 37° C., and the like, and for passage, for example, the embryonic stem cell line is rendered to be single cells by a treatment with a trypsin/EDTA solution (usually 0.001 to 0.5% trypsin/0.1 to 5 mM EDTA, preferably about 0.1% trypsin/1 mM EDTA), and seeded onto freshly provided feeder cells, and the like, can be used. This passage is usually conducted every 1 to 3 days, during which period the cells are examined; if a morphologically abnormal cell is found, the cultured cells are desirably discarded.

ES cells can be differentiated into various types of cells,including-parietal muscle, visceral muscle, cardiac muscle and the like, by monolayer culture until a high density is obtained, or by suspension culture until a cell aggregation is formed, under appropriate conditions [M. J. Evans and M. H. Kaufman, Nature, Vol. 292, p. 154, 1981; G. R. Martin, Proc. Natl. Acad. Sci. U.S.A., Vol. 78, p. 7634, 1981; T. C. Doetschman et al., Journal of Embryology and Experimental Morphology, Vol. 87, p. 27, 1985], and non-human mammal cell expressing the gene, obtained by differentiating the ES cell transferred with a gene under control of a promoter comprising the FRE of the present invention, is useful for determining responsiveness of the FRE of the present invention on diet (e.g., high-fructose diet) in vitro.

For gene transfer to an ES cell, any of the calcium hosphate co-precipitation method, electric pulse (electroporation) method, lipofection method, retrovirus infection method, aggregation method, microinjection method, gene gun (particle gun) method, DEAE-dextran method and the like can be used; however, the electroporation method is generally chosen for the reasons of the capability of treating a large number of cells conveniently and the like. For electroporation, ordinary conditions used for gene transfer to animal cells can be used as is; for example, electroporation can be conducted by trypsinizing ES cells in the logarithmic growth phase to disperse them to obtain a dispersion of single cells, suspending the dispersion in a medium to obtain a cell density of 10⁶ to 10⁸ cells/ml, transferring the suspension to a cuvette, adding 10 to 100 μg of a vector comprising a transferred DNA, and applying electric pulses of 200 to 600 V/cm.

Although the ES cell incorporating the transferred DNA can also be tested by screening chromosome DNAs separated and extracted from a colony obtained by culturing a single cell on feeder cells, by Southern hybridization or PCR method, the greatest advantage of a transgenic system using an ES cell resides in that a transformant can be selected at the cell stage with the expression of a drug resistance gene or a reporter gene as the index. Therefore, the transfer vector used here desirably further comprises, in addition to an expression cassette for a gene under the control of a promoter comprising FRE of the present invention, a selection marker gene such as a drug resistance gene (e.g., neomycin phosphotransferase II (nptII) gene, hygromycin phosphotransferase (hpt) gene and the like) or a reporter gene (e.g., β-galactosidase (lacZ) gene, chloramphenicol acetyltransferase (cat) gene and the like). For example, when using a vector comprising the nptII gene as the selection marker gene, the ES cell after gene transfer treatment is cultured in a medium containing a neomycin series antibiotic, such as G418, each of the emerging resistant colonies is transferred to a culture plate; after trypsinization and medium exchanges are repeated, a portion thereof is reserved for cultivation, whereas the remainder is subjected to PCR or Southern hybridization to confirm the presence of the transferred DNA.

When an ES cell confirmed to have the transferred DNA. incorporated therein is returned into an embryo derived from the same species of non-human mammal, it is incorporated in the ICM of the host embryo and a chimeric embryo is formed. By transplanting this to a foster parent (a female for embryo reception), and allowing development to continue, a chimeric transgenic animal is obtained. If the ES cell has contributed to the formation of primordial germ cells, which will differentiate into eggs and sperm in the chimeric animal, a germ line chimera would be obtained; by mating this, a transgenic non-human mammal having the transferred DNA fixed genetically therein can be prepared.

As the method of preparing a chimeric embryo, there are a method wherein early embryos up to the morula stage are adhered together and aggregated (aggregation chimera method) and a method wherein a cell is microinjected into a cleavage cavity of the blastocyst (injection chimera method). Although the latter has traditionally been widely conducted in the preparation of a chimeric embryo using an ES cell, a method wherein an aggregate chimera is created by injecting an ES cell into the zona pellucida of an 8-cell stage embryo, and a method wherein an aggregate chimera is created by co-culturing and aggregating an ES cell mass and an 8-cell stage embryo having the zona pellucida removed therefrom as a method which does not require a micromanipulator and which can be easily operated, have recently also been conducted.

In all cases, a host embryo can be collected in the same manner from a non-human mammal that can be used as the female for egg collection in gene transfer to a fertilized egg; for example, in the case of the mouse, to enable a determination of the percent contribution of the ES cell to chimeric mouse formation by fur color (coat color), it is preferable to collect a host embryo from a mouse of a strain whose fur color is different from that of the strain from which the ES cell is derived. For example, provided that the ES cell is derived from a 129 strain mouse (fur color: agouti), a C57BL/6 mouse (fur color: black) and an ICR mouse (fur color: albino) can be used as the female for egg collection; provided that the ES cell is derived from a C57BL/6 or DBF₁ mouse (fur color: black) or the TT2 cell (F₁ of C57BL/6 and CBA (fur color: agouti)), an ICR mouse or a BALB/c mouse (fur color: albino) can be used as the female for egg collection.

Also, because the germ line chimera formation potential depends largely on the combination of ES cell and host embryo, it is more preferable to select a combination showing a high germ line chimera formation potential. For example, in the case of the mouse, it is preferable to use a host embryo and the like derived from the C57BL/6 strain for ES cells derived from the 129 strain, and host embryo and the like derived from the BALB/c strain are preferred for ES cells derived from the C57BL/6 strain.

The female mice for egg collection are preferably about 4 to about 6 weeks or so of age; as the male mouse for mating, one of the same strain at about 2 to about 8 months or so of age is preferred. Although mating may be by spontaneous mating, it is preferably conducted after gonadotropic hormones (follicle-stimulating hormone, then luteinizing hormone) are administered to induce superovulation.

In the case of the blastodisk injection method, a blastocystic embryo (for example, in the case of a mouse, at about 3.5 days after mating) is collected from the uterus of a female for egg collection (or an early embryo in the morula stage or before, after being collected from the oviduct, may be cultured in the above-described medium for embryo culture until the blastocyst stage), and an ES cell having a gene under control of a promoter comprising the FRE of the present invention transferred thereto (about 10 to about 15 cells) is injected into a cleavage cavity of the blastocyst using a micromanipulator, after which it is transplanted into the uterus of a female non-human mammal for embryo reception rendered to be pseudopregnant. As the female non-human mammal for embryo reception, a non-human mammal which can be used as a female for embryo reception in gene transfer to a fertilized egg, can be used in the same manner.

In the case of the co-culture method, an 8-cell stage embryo and morula (for example, in the case of the mouse, about 2.5 days after mating) are collected from the oviduct and uterus of the female for egg collection (or it is also possible to collect an early embryo in the 8-cell stage or before from the oviduct, and thereafter culture it in the above-described medium for embryo culture until the 8-cell stage or the morula stage); after the zona pellucida is dissolved in acidic Tyrode's solution, an ES cell mass having a gene under control of a promoter comprising the FRE of the present invention transferred thereto (number of cells: about 10 to about 15 cells) is placed in a droplet of a medium for embryo culture overlain with mineral oil, the above-described 8-cell stage embryo or morula (preferably 2 cells) is further placed, and they are co-cultured overnight. The obtained morula or blastocyst is transplanted into the uterus of a female non-human mammal for embryo reception in the same manner as above.

If the transplanted embryo successfully implants and the embryo recipient female becomes pregnant, chimeric non-human mammals are obtained by spontaneous delivery or caesarian section. Embryo recipient females that delivered spontaneously are allowed to continue suckling; if the female has delivered by caesarian section, the pups may be suckled by a separately provided female for suckling (a female non-human mammal with usual mating and delivery).

For selection of a germ line chimera, first, a chimeric mouse, of the same sex as the ES cell, provided that the sex of the ES cell is determined in advance, is selected (usually, a male chimeric animal is selected because a male ES cell is used), next, a chimeric animal showing a high percent contribution of the ES cell (for example, 50% or higher), based on a phenotype such as fur color, is selected. For example, in the case of a chimeric mouse obtained from a chimeric embryo of the D3 cell, which is a male ES cell derived from the 129 strain mouse, and a host embryo derived from a C57BL/6 mouse, it is preferable to select a male mouse showing a high percentage of the agouti fur color. Whether or not the, selected chimeric non-human mammal is a germ line chimera can be determined on the basis of the phenotype of the F₁ animal obtained by crossing with the same species of animal of the appropriate strain. For example, in the case of the above-described chimeric mouse, agouti is dominant over black; therefore, when the selected male mouse is crossed with a female C57BL/6 mouse, the fur color of the obtained F₁ would be agouti, provided that the male is a germ line chimera.

The thus-obtained germ line chimeric non-human mammal (founder) incorporating a gene under the control of a promoter comprising the FRE of the present invention is usually obtained as a heterozygote having the transferred DNA only in one of the homologous chromosomes. Also, the individual founders are randomly inserted onto different chromosomes unless based on homologous recombination. To obtain a homozygote having a gene under the control of a promoter comprising the FRE of the present invention on both of the homologous chromosomes, among F₁ animal obtained as described above, heterozygous siblings having the transferred DNA only in one of the homologous chromosomes are crossed. Selection of heterozygotes can, for example, be tested by screening chromosome DNA separated and extracted from the tail of the F₁ animal by Southern hybridization or PCR method. Provided that the transferred DNA has been incorporated in only one gene locus, one-fourth of the obtained F₂ animals would be homozygotes.

The FRE-Tg animal of the present invention is useful in examining the expressional response of a gene under the control of a promoter comprising the FRE of the present invention (for example, SREBP-1c gene) to sugars (especially fructose) loading. Although no problem arises if the structural gene used as the transgene is a reporter gene not inherently present in the host animal; for example, when using the SREBP-1c gene as a structural gene (especially when the SREBP-1c gene comprising the FRE of the present invention in the promoter region thereof is used as a “gene under the control of a promoter comprising the FRE of the present invention”), it is desirable that the endogenous SREBP-1c gene be inactivated. The FRE-Tg animal of the present invention having the endogenous SREBP-1c gene inactivated (the SREBP-1c gene knock-out animal) can be obtained by introducing a gene according to the method described above to an ES cell having the SREBP-1c gene knocked out, selected by a publicly known method (see, for example, Lee S. S. et al., Molecular and Cellular Biology, Vol. 15, page 3012, 1995), or an early embryo or ES cell derived from a SREBP-1c knock-out animal prepared from the ES cell according to the method described above. As a specific means of knocking out a SREBP-1c gene, a method wherein the SREBP-1c gene derived from the subject non-human mammal is isolated according to a conventional method, and a DNA chain having a DNA sequence constructed so that the gene is eventually inactivated by, for example, inserting another DNA fragment (for example, drug resistance genes such as, the neomycin resistance gene and the hygromycin resistance gene, reporter genes such as lacZ (β-galactosidase gene) and cat (chloramphenicol acetyltransferase gene) and the like) to the exon portion thereof to destroy the function of the exon (in this case, incorporation of the transferred DNA can be selected with drug resistance or reporter gene expression as the index as described above), or by cleaving out all or a portion of the SREBP-1c gene using the Cre-loxP sy stem or the Flp-frt system to delete the gene, or by inserting the stop codon into the protein-coding region to disable the complete translation of the protein, or by inserting a DNA sequence that terminates the transcription of the gene (for example, polyA addition signal and the like) into the transcription region to disable the complete synthesis of the messenger RNA (hereinafter abbreviated as targeting vector), is incorporated by homologous recombination into the SREBP-1c gene locus of the subject non-human mammal, can be preferably mentioned.

Usually, gene recombinations in a mammal are mostly non-homologous; the transferred DNA is randomly inserted at an optionally chosen position on the chromosome. Therefore, it is not possible to efficiently select only those clones targeted to the target endogenous SREBP-1c gene by homologous recombination by selection based on detection of drug resistance or reporter gene expression and the like; it is necessary to confirm the incorporation site by the Southern hybridization method or the PCR method for all selected clones. Hence, provided that, for example, the herpes simplex virus-derived thymidine kinase (HSV-tk) gene, which confers gancyclovir susceptibility, has been joined outside of a region homologous to the target sequence of the targeting vector, the cells having the vector inserted randomly thereto are incapable of growing in a gancyclovir containing medium because they have the HSV-tk gene, whereas the cells that have been targeted to the endogenous SREBP-1c gene locus by homologous recombination become resistant to gancyclovir and are selected because they do not have the HSV-tk gene. Alternatively, provided that the diphtheria toxin gene, for example, is joined in place of the HSV-tk gene, the cells having the vector inserted randomly thereto die due to the toxin produced thereby, so that a homologous recombinant can also be selected in the absence of a drug.

Alternatively, the transgenic animal of the present invention having the expression of an endogenous SREBP-1c gene inactivated maybe a knock-in animal wherein an endogenous SREBP-1c gene is substituted by an SREBP-1c gene comprising the FRE of the present inventionin the promoter region thereof by gene targeting using homologous recombination. That is, the present invention also provides an FRE-Tg animal wherein an endogenous SREBP-1c gene not having the FRE of the present invention in a promoter region (for example, comprising the mutated FRE of the present invention in the promoter region) is substituted by the SREBP-1c gene under the control of a promoter comprising the FRE of the present invention.

As examples of non-human host animals having an endogenous SREBP-1c gene not comprising the FRE of the present invention in the promoter region thereof, a non-human animal having the mutated FRE of the present invention in the promoter region thereof can be mentioned; specifically, a non-human animal strain that does not increase the expression of the SREBP-1c gene in response to sugar loading (especially fructose loading), or does not have a tendency for a metabolic disorder such as increased serum lipid (for example, C57BL and DBA strain mice, in the case of mice) can be mentioned.

A knock-in animal can be prepared according to basically the same technique as that for a knock-out animal. A targeting vector comprising an SREBP-1c gene under the control of a promoter comprising the FRE of the present invention is transferred to an ES cell derived from a subject non-human mammal according to the above-described method, and an ES cell clone having the SREBP-1c gene under the control of the promoter comprising the FRE of the present invention incorporated by homologous recombination in the animal's endogenous SREBP-1c gene locus is selected. Clone selection can be conducted using the PCR method or the Southern hybridization method; for example, provided that a marker gene for positive selection such as the neomycin resistance gene is inserted into a 3′ non-translational region of the SREBP-1c gene in the targeting vector and the like, and that a marker gene for negative selection such as the HSV-tk gene or the diphtheria toxin gene is inserted outside a region homologous to the target sequence, a homologous recombinant can be selected with drug resistance as an index.

Since there are some cases wherein the expression of SREBP-1c incorporating a marker gene for positive selection is prevented, it is preferable that a marker gene for positive selection be cleaved out by reacting a Cre or Flp recombinase or an expression vector for the recombinase (e.g., adenovirus vector and the like) with a targeting vector wherein the loxP sequence or frt sequence is placed at both ends of the marker gene for positive selection, at an appropriate time following homologous recombinant selection. Alternatively, in place of using the Cre-loxP system or the Flp-frt system, a sequence homologous to the target sequence may be repeatedly placed in the same direction at both ends of the marker gene for positive selection, and the marker gene for positive selection may be cleaved out by making use of recombination in the gene among the sequences.

The thus-obtained transgenic animal of the present invention having an SREBP-1c gene with a substituted promoter (hereinafter referred to as “the FRE-KI animal of the present invention”) has the fructose responsive element of the present invention in the SREBP-1c promoter region. As shown in Examples below, in the hepatocytes of DBA/2 mouse, the expression of the transcriptional regulatory factor of the present invention capable of binding to the FRE of the present invention was suppressed whether during fasting or after meals. Therefore, by examining the expression of the transcriptional regulatory factor of the present invention in the FRE-KI animal of the present invention, differences in the expression of the transcriptional regulatory factor among various animal strains may be elucidated, and in turn the mechanism of control of the expression of genes associated with sugar or lipid metabolism, including the transcriptional regulatory factor and the SREBP-1c gene, may be clarified in full.

For the same purpose as above, the present invention provides a non-human transgenic animal incorporating a gene under the control of a promoter comprising the mutated FRE of the present invention, or a transgenic animal wherein an endogenous SREBP-1c gene comprising the FRE of the present invention in the promoter region thereof is substituted by an SREBP-1c gene not having the FRE of the present invention in the promoter region thereof (for example, the SREBP-1c gene under the control of a promoter comprising the mutated FRE of the present invention).

A non-human transgenic animal incorporating a gene under the control of a promoter comprising the mutated FRE of the present invention can be prepared in the same manner as the above-described FRE-Tg animal of the present invention, except that the mutated FRE of the present invention is used in place of the FRE of the present invention.

As examples of the host non-human animal having an endogenous SREBP-1c gene comprising the FRE of the present invention in the promoter region thereof, a non-human animal strain showing a tendency for increased expression of SREBP-1c gene or a metabolic disorder such as increased serum lipid in response to sugar loading (especially fructose loading) (for example, CBA and C3H strain mice, in the case of mice) can be mentioned.

A knock-in animal can be prepared in the same manner as the above-described FRE-KI animal of the present invention.

The present invention also provides a non-human transgenic animal incorporating a DNA that encodes the transcriptional regulatory factor of the present invention, and a non-human animal (knock-out animal) having the DNA that encodes the transcriptional regulatory factor inactivated.

Such transgenic animals and knock-out animals can be prepared by the same method as the above-described FRE-Tg animal of the present invention and the SREBP-1c gene knock-out animal.

A non-human transgenic animal transferred with the DNA (normal DNA) encoding the transcriptional regulatory factor of the present invention (hereinafter, referred to as a “TF-Tg animal of the present invention”) has the transcriptional regulatory factor of the present invention expressed at a high level therein, possibly finally develops hyperfunction of the transcriptional regulatory factor by promoting the function of endogenous normal DNA, and can be utilized as a pathologic model animal thereof. For example, using the normal DNA transferred animal of the present invention, it is possible to elucidate the pathologic mechanism of the hyperfunction of the transcriptional regulatory factor of the present invention or disease associated with the transcriptional regulatory factor, and to investigate a therapeutic method for these diseases.

Also, because the TF-Tg animal of the present invention has a symptom in which the transcriptional regulatory factor of the present invention is increased, it can also be utilized for a screening test for a prophylactic or therapeutic substance for a disease associated with increased function of the transcriptional regulatory factor, for example, metabolic disorder, especially glucose or lipid metabolic disorder (e.g., hypertriglyceridemia, hyper-LDL-cholesteremia, hypo-HDL-cholesterolemia, obesity, abnormality of glucose tolerance, fasting blood glucose disorder, hyperinsulinemia, hypertension, albuminuria, and the like) and the like.

On the other hand, a non-human mammal having DNA (abnormal DNA) that encodes abnormal transcriptional regulatory factor of the present invention (i e., mutant protein of the transcriptional regulatory factor of the present invention, which does not show transcriptional function) (hereinafter, referred to as an “abnormal TF-Tg animal of the present invention”) has the abnormal DNA of the present invention expressed at a high level therein, possibly finally develops function inactivation type unresponsiveness of the transcriptional regulatoryfactor of the present invention by inhibiting the function of endogenous normal DNA (for example, transcription promoting activity on the SREBP-1c gene and the like), and can be utilized as' a pathologic model animal thereof. For example, using the abnormal TF-Tg animal of the present invention, it is possible to elucidate the pathologic mechanism of function inactivation type unresponsiveness of the transcriptional regulatory factor, and to investigate a therapeutic method for this disease.

Additionally, as the specific availability, the abnormal TF-Tg animal of the present invention can serve as a model for elucidating the inhibition of the function of normal transcriptional regulatory factor by abnormal transcriptional regulatory factor in function inactivation type unresponsiveness of the transcriptional regulatory factor of the present invention (dominant negative action).

Also, because the abnormal TF-Tg animal of the present invention has a symptom in which the function of the transcriptional regulatory factor of the present invention is inhibited, it can also be utilized for a screening test for a therapeutic drug for function inactivation type unresponsiveness of the transcriptional regulatory factor.

Furthermore, using the TF-Tg animal of the present invention, it is possible to provide an effective and quick screening method for a prophylactic or therapeutic agent for a disease associated with the transcriptional regulatory factor of the present invention, including function inactivation type unresponsiveness of the transcriptional regulatory factor, to develop the prophylactic or therapeutic agent using the above-described test method, quantitation method and the like. It is also possible to investigate and develop a gene therapy method for a disease associated with the transcriptional regulatory factor of the present invention, using the TF-Tg animal of the present invention or the DNA expression vector that encodes the transcriptional regulatory factor of the present invention.

The non-human mammal whose DNA encoding the transcriptional regulatory factor of the present invention is inactivated: (hereinafter, abbreviated as a “TF-KO animal of the present invention”) can be used to screen for a compound having a therapeutic or prophylactic effect on a disease caused by deficiency, damage or the like of the DNA encoding the transcriptional regulatory factor of the present invention.

Accordingly, the present invention provides a screening method for a compound having a therapeutic or prophylactic effect on a disease caused by deficiency, damage and the like of the DNA that encodes the transcriptional regulatory factor of the present invention, or a salt thereof, which comprises administering a test compound to the TF-KO animal of the present invention, and examining and measuring the changes in the animal.

As examples of the test compound, a peptide, a protein, a non-peptide compound, a synthetic compound, a fermentation product, a cell extract, a plant extract, an animal tissue extract, plasma and the like can be mentioned, and these compounds may be novel compounds or publicly known compound.

Specifically, the therapeutic or prophylactic effect of a test compound can be tested by administering the test compound to the TF-KO animal of the present invention, and comparing the changes in various organs, tissues, disease symptoms and the like in the animal with control animals not administered with the test compound.

As examples of the method of administering the test compound to the TF-KO animal, oral administration, intravenous injection and the like can be used, and the method can be selected as appropriate for the TF-KO animal's symptoms, test compound nature and the like. Also, the dosage of the test compound can be appropriately selected according to method of administration, nature of the test compound, and the like.

In the screening method, when the TF-KO animal is administered with a test compound, and, for example, if the TF-KO animal's symptoms have improved by about 10% or more, preferably about 30% or more, more preferably about 50% or more, the test compound can be selected as a compound that has a therapeutic or prophylactic effect on the above-described disease.

The compound obtained using the screening method is a compound selected from among the above-described test compounds, and can be used as a pharmaceutical such as a therapeutic or prophylactic agent and the like that is safe and of low toxicity for the disease caused by deficiency, damage and the like of the transcriptional regulatory factor of the present invention. Furthermore, a compound derived from a compound obtained by the above-described screening can also be used in the same manner.

The compound obtained by the screening method may have formed a salt; as the salt of the compound, physiologically acceptable salts with acid (for example, inorganic acids, organic acids and the like) or base (for example, alkaline metals and the like) can be used, and physiologically acceptable acid addition salts are particularly preferred. Useful salts include, for example, salts with inorganic acids (for example, hydrochloric acid, phosphoric acid, hydrobromic acid, sulfuric acid and the like) or salts with organic acids (for example, acetic acid, formic acid, propionic acid, fumaric acid, maleic acid, succinic acid, tartaric acid, citric acid, malic acid, oxalic acid, benzoic acid, methanesulfonic acid, benzenesulfonic acid and the like) and the like.

A pharmaceutical containing the compound obtained by the screening method or a salt thereof can be formulated into a preparation and administered to a mammal in the same manner as the aforementioned inhibitor of the transcriptional regulatory factor of the present invention.

The present invention provides a screening method for a compound that promotes or inhibits the promoter activity of the gene of the transcriptional regulatory factor of the present invention, or a salt thereof, which comprises administering a test compound to the TF-KO animal of the present invention, and detecting the expression of a reporter gene.

In the above-described screening method, as the TF-KO animal of the present invention, one having the gene of the transcriptional regulatory factor of the present invention inactivated by tranfer of a reporter gene, which reporter gene is expressible under the control of a promoter for the gene of the transcriptional regulatory factor of the present invention, can be used.

As the test compound, the same as those mentioned above can be mentioned.

As the reporter gene, for example, the β-galactosidase gene (lacZ), the soluble alkaline phosphatase gene or the luciferase gene and the like are preferred.

In the TF-KO animal of the present invention wherein the gene of the transcriptional regulatory factor of the present invention is substituted by a reporter gene, the activity of the promoter can be detected by tracing the expression of the substance encoded by the reporter gene because the reporter gene is present under the control of the promoter of the gene of the transcriptional regulatory factor of the present invention.

For example, when a portion of the DNA region that encodes the transcriptional regulatory factor of the present invention has been replaced by the Escherichia coli-derived β-galactosidase gene (lacZ), β-galactosidase is expressed, in place of the transcriptional regulatory factor of the present invention, in tissues where the transcriptional regulatory factor of the present invention is expressed originally. Therefore, the expression state of the transcriptional regulatory factor of the present invention within the animal body can be conveniently observed by, for example, staining with a reagent that can serve as a substrate for β-galactosidase, like 5-bromo-4-chloro-3-indolyl-β-galactopyranoside (X-gal). Specifically, the expression state can be observed by fixing a mouse lacking the transcriptional regulatory factor of the present invention or a tissue section thereof with glutaraldehyde and the like, washing with phosphate-buffered saline (PBS), carrying out the reaction with a staining solution containing X-gal at room temperature or at nearly 37° C. for about 30 minutes to 1 hour, washing the tissue specimen with 1 mM EDTA/PBS solution to stop the β-galactosidase, reaction, and examining the color developed. Also, the mRNA that encodes lacZ may be detected according to a conventional method.

A compound obtained using the above-described screening method or a salt thereof is a compound selected from among the above-described test compounds, that promotes or inhibits the promoter activity for the transcriptional regulatory factor of the present invention.

The compound obtained by the screening method may have formed a salt, and as the salt of the compound, physiologically acceptable salts with acid (for example, inorganic acids and the like) or base (for example, organic acids and the like) and the like can be used, and physiologically acceptable acid addition salts are particularly preferred. Useful salts include, for example, salts with inorganic acids (for example, hydrochloric acid, phosphoric acid, hydrobromic acid, sulfuric acid and the like) or salts with organic acids (for example, acetic acid, formic acid, propionic acid, fumaric acid, maleic acid, succinic acid, tartaric acid, citric acid, malic acid, oxalic acid, benzoic acid, methanesulfonic acid, benzenesulfonic acid and the like) and the like.

Because a compound that promotes the promoter activity for the gene of the transcriptional regulatory factor of the present invention or a salt thereof is capable of promoting the expression of the transcriptional regulatory factor of the present invention and of promoting the function of the transcriptional regulatory factor of the present invention, it can be used as a pharmaceutical such as a prophylactic or therapeutic agent for, for example, a disease associated with functional impairment of the transcriptional regulatory factor of the present invention and the like.

On the other hand, because a compound that inhibits the promoter activity for the gene of the transcriptional regulatory factor of the present invention or a salt thereof is capable of inhibiting the expression of the transcriptional regulatory factor of the present invention and of inhibiting the function of the transcriptional regulatory factor of the present invention, it is useful as a pharmaceutical such as a prophylactic or therapeutic agent for, for example, a disease associated with overexpression of the transcriptional regulatory factor of the present invention and the like. Specifically, the compound can be used as a safe pharmaceutical of low toxicity such as a prophylactic or therapeutic agent for a disease, for example, metabolic disorder, especially glucose or lipid metabolic disorder (e.g., hypertriglyceridemia, hyper-LDL-cholesteremia, hypb-HDL-cholesterolemia, obesity, abnormality of glucose tolerance, fasting blood glucose disorder; hyperinsulinemia, hypertension, albuminuria, and the like), and the like.

Furthermore, a compound derived from a compound obtained by the above-described screening can also be used in the same manner.

A pharmaceutical, containing the compound obtained by the screening method or a salt thereof can be formulated into a preparation and administered to a mammal in the same manner as the aforementioned inhibitor of the transcriptional regulatory factor of the present invention.

As described above, the TF-KO animal of the present invention is very useful in screening for a compound that promotes or inhibits thepromoter activity for the gene of the transcriptional regulatory factor of the present invention or a salt thereof, and can significantly contribute to the elucidation the causes of various diseases due to expression deficiency of the DNA that encodes the transcriptional regulatory factor of the present invention, or the development of prophylactic or therapeutic agent for the same.

Additionally, provided that, using a DNA comprising the promoter region of the gene of thetranscriptional regulatory factor of the present invention, genes that encode various proteins are joined downstream of the promoter region, and the DNA is injected to an animal ovum to prepare what is called a transgenic animal (gene transferred animal), it is possible to allow the animal to tissue- and/or time-specifically synthesize the protein, and investigate its action in the body. Furthermore, provided that the above-described promoter portion is bound with an appropriate reporter gene, and a cell line that allows its expression is established, the cell line can be used as a screening system for a low-molecular compound that acts to specifically promote or suppress the producibility of the transcriptional regulatory factor of the present invention itself in the body.

Abbreviations for bases, amino acids and the like used in the present specification and drawings are based on abbreviations specified by the IUPAC-IUB Commission on Biochemical Nomenclature or abbreviations in common use in relevant fields. Some examples are given below. When an enantiomer may be present in amino acid, it is of the L-configuration, unless otherwise stated.
DNA: Deoxyribonucleic acid
cDNA: Complementary deoxyribonucleic acid
A: Adenine
T: Thymine
G: Guanine
C: Cytosine
RNA: Ribonucleic acid
mRNA: Messenger ribonucleic acid
dATP: Deoxyadenosine triphosphate
dTTP: Deoxythymidine triphosphate
dGTP: Deoxyguanosine triphosphate
dCTP: Deoxycytidine triphosphate
ATP: Adenosine triphosphate
EDTA: Ethylenediaminetetraacetic acid
SDS: Sodium dodecyl sulfate
Gly: Glycine
Ala: Alanine
Val: Valine
Leu: Leucine
Ile: Isoleucine
Ser: Serine
Thr: Threonine
Cys: Cysteine
Met: Methionine
Glu: Glutamic acid
Asp: Aspartic acid
Lys: Lysine
Arg: Arginine
His: Histidine
Phe: Phenylalanine
Tyr: Tyrosine
Trp: Tryptophan
Pro: Proline
Asn: Asparagine
Gln: Glutamine
pGlu: Pyroglutamic acid
*: Corresponds to stop codon.
Me: Methyl group
Et: Ethyl group
Bu: Butyl group
Ph: Phenyl group
TC: Thiazolidine-4(R)-carboxamide group

Substituents, protecting groups and reagents frequently mentioned herein are represented by the symbols shown below.
Tos: p-Toluenesulfonyl
CHO: Formyl
Bzl: Benzyl
Cl₂Bzl: 2, 6-Dichlorobenzyl
Bom: Benzyloxymethyl
Z: Benzyloxycarbonyl
Cl-Z: 2-Chlorobenzyloxycarbonyl
Br-Z: 2-Bromobenzyloxycarbonyl
Boc: t-Butoxycarbonyl
DNP: Dinitrophenol
Trt: Trityl
Bum: t-Butoxymethyl
Fmoc: N-9-Fluorenylmethoxycarbonyl
HOBt: 1-Hydroxybenztriazole
HOOBt: 3,4-Dihydro-3-hydroxy-4-oxo-1,2,3-benzotriazine
HONB: 1-Hydroxy-5-norbornane-2,3-dicarboximide
DCC: N, N′-Dicyclohexylcarbodiimide

The sequence identification numbers in the sequence listing herein show the following sequences. [SEQ ID NO:1]

shows the base sequence of the SREBP-1c promoter region (from immediately before the deduced transcriptional starting site to the upstream of 574^{th} base) derived from CBA strain mouse. [SEQ ID NO:2]

shows the base sequence encoding the NBP derived from mouse. [SEQ ID NO:3]

shows the amino acid sequence of the NBP derived from mouse. [SEQ ID NO:4]

shows the base sequence encoding the RBMX analogous protein derived from mouse. [SEQ ID NO:5]

shows the amino acid sequence of the RBMX analogous protein derived from mouse. [SEQ ID NO:6]

shows the base sequence of the SREBP-1c promoter region (from TATA-like sequence to the upstream of about 1.2 kb) derived from CBA mouse. [SEQ ID NO:7]

shows the base sequence of an oligonucleotide designed as a primer for amplifying the SREBP-1c promoter region (from TATA-like sequence to the upstream of about 1.2 kb). [SEQ ID NO:8]

shows the base sequence of an oligonucleotide designed as a primer for amplifying the SREBP-1c promoter region (from TATA-like sequence to the upstream of about 1.2 kb). [SEQ ID NO:9]

shows the base sequence of an oligonucleotide corresponding to the fructose responsive element in the SREBP-1c promoter from CBA/JN Crj mouse. [SEQ ID NO:10]

shows the base sequence of an oligonucleotide corresponding to the mutated fructose responsive element in the SREBP-1c promoter-from DBA/2 JN Crj mouse. [SEQ ID NO:11]

shows the base sequence of an oligonucleotide corresponding to a sense strand of the fructose responsive element in the SREBP-1c promoter from CBA/JN Crj mouse. [SEQ ID NO:12]

shows the base sequence of an oligonucleotide corresponding to an antisense strand of the fructose responsive element in the SREBP-1c promoter from CBA/JN Crj mouse. [SEQ ID NO:13]

shows the base sequence of the SREBP-1c promoter region [corresponding to complementary strand sequence of the base sequence represented by base number 16566061-16566560in the base sequence of human chromosome 17 (accession number: NT_{—}010718) registered in GenBank] derived from human.

The present invention is hereinafter described in more detail by means of the following Examples, which examples, however, are not to be construed as limiting the scope of the present invention.

All materials used were of reagent grade, and were purchased from Nacalai Tesque or Sigma Chemical unless otherwise specified. Numerical data are shown as mean±standard deviation unless otherwise specified. Significant differences among the groups were determined using the Tukey-Welsh tapering multiple comparison. P<0.05 was considered to indicate statistical significance.

### EXAMPLE 1

Comparison of Metabolic Responses of Various Mouse Strains to Feeding

Five different inbred strains [BALB/c Cr Slc (purchased from Japan SLC, Inc.); C3H/HeJ (purchased from Charles River Japan); C57BL/6J Jcl, DBA/2N Crj and CBA/JN Crj (all purchased from Clea Japan, Inc.)] of mice (5-weeks old, male) were placed in a animal room with a 12-hour light/dark cycle and allowed to have free access to a laboratory diet and water.

The animals were divided into two groups (an ordinary diet group and a high-fructose diet group) and concurrently fed for 8 weeks. The ordinary diet (Oriental Yeast Co., Ltd.) consisted of 58% carbohydrate (fructose not contained), 12% lipid and 30% protein, and the high-fructose diet (Oriental Yeast Co., Ltd.) consisted of 67% carbohydrate (98% accounted for by fructose), 13% lipid and 20% protein (% values are shown as % calorie).

The day before the experiment, all animals were deprived food at 8:00 p.m. Subsequently, the animals were divided into four groups (4-8 animals per group): 1) animals fed the ordinary diet without refeeding (control-fasted; CF), 2) animals fed the ordinary diet with refeeding (control-refeeding; CR), 3) animals fed the high-fructose diet without refeeding (fructose-fasted; FF), and 4) animals fed the high-fructose diet with refeeding (fructose-refeeding; FR). The mice in the CR group and the FR group were re-fed in the dark between 6:00 a.m. and 8:00 a.m. The CF group and the FF group continued to be fasted. After the body weight (BW) of each mouse was measured, the mouse was anesthetized and laparotomized at 10:00 a.m., the liver and epididymis fat were resected, and the weight of epididymis fat (FW) was measured. Also, various blood tests for blood sugar (BS), triglycerides (TG), total cholesterol (CHO), and insulin (INS) were performed according to conventional methods. The results from the CR group are shown in Table 1, and the results from the FR group are shown in Table 2.

TABLE 1

| Mouse | BW | FW | BS | TG | CHO | INS |
|---|---|---|---|---|---|---|
| strain | (g) | (g) | (mg/dl) | (mg/dl) | (mg/dl) | (ng/ml) |
| C3H/HeJ | 23 ± 2 | 0.37 ± 0.11 | 288 ± 31 | 154 ± 12 | 90 ± 7 | 0.3 ± 0.01 |
| C57BL/6J | 23 ± 0 | 0.3 ± 0 | 302 ± 1 | 103 ± 4 | 63 ± 1 | 0.1 ± 0 |
| BALB/c Cr SLC | 21 ± 2 | 0.53 ± 0.01 | 269 ± 7 | 106 ± 15 | 240 ± 7 | 1.3 ± 0.3 |
| CBA/JN Crj | 28 ± 2 | 0.75 ± 0.07 | 252 ± 8 | 262 ± 35 | 77 ± 2 | 1.2 ± 0.4 |
| DBA/2JN Crj | 23 ± 1 | 0.31 ± 0.06 | 223 ± 22 | 118 ± 9 | 91 ± 3 | 0.1 ± 0 |

TABLE 2

| Mouse | BW | FW | BS | TG | CHO | INS |
|---|---|---|---|---|---|---|
| strain | (g) | (g) | (mg/dl) | (mg/dl) | (mg/dl) | (ng/ml) |
| C3H/HeJ | 33 ± 1^{a} | 1.06 ± 0.12^{b} | 328 ± 30 | 133 ± 16 | 153 ± 16 | 2.3 ± 0^{b} |
| C57BL/6J | 23 ± 0 | 0.5 ± 0^{b} | 301 ± 30 | 101 ± 1 | 113 ± 3^{a} | 0.1 ± 0 |
| BALB/c Cr SLC | 27 ± 1 | 0.77 ± 0.3 | 317 ± 7^{a} | 153 ± 28 | 316 ± 7^{a} | 2.4 ± 0^{b} |
| CBA/JN Crj | 31 ± 1 | 1.41 ± 0.12^{a} | 331 ± 19^{a} | 392 ± 43^{a} | 113 ± 7^{b} | 6.5 ± 0.2^{b} |
| DBA/2JN Crj | 25 ± 1 | 0.5 ± 0.13 | 242 ± 15 | 160 ± 27 | 110 ± 4 | 0.2 ± 0 |

^{a}p < 0.05 vs CR;
^{b}p < 0.01 vs CR

In the CBA/JN Crj mice, epididymis fat weight, blood sugar level, TG, CHO, and INS significantly increased with the high-fructose diet compared to the ordinary diet; whereas in DBA/2JN Crj mice, no significant difference was observed between the ordinary diet and the high-fructose diet.

### EXAMPLE 2

Correlation between Metabolic Responses of Various Mouse Strains to Feeding and SREBP-1c Promoter Sequence

After five different inbred strains [C3H/He Slc (purchased from Japan SLC, Inc.); C57BL/6N Jcl, DBA/1JN Crj, DBA/2N Crj and CBA/JN Crj (all purchased from Clea Japan, Inc.)] of mice (5-weeks old, male) were raised using the same feeding method as Example 1 (4-6 animals per group), they were anesthetized at 10:00 a.m. and the livers were resected, immediately frozen in liquiid nitrogen and stored at −80° C. Also, serum triglyceride (TG) concentrations were measured according to a conventional method.

Separately, genomic DNA was isolated from each frozen liver using a DNeasy kit (QIEGEN). Primers (sense: 5′-GCTGGACAGAACGGTGTCAT-3′ (SEQ ID NO:7); antisense: 5′-TAAGAGCTCGGTACCTCCCCTAGGGC-3′ (SEQ ID NO:8)) were synthesized on the basis of the publicly known mouse SREBP-1c promnoter sequence, and PCR was conducted with each genomic DNA as a template to amplify an about 1.2 kb SREBP-1c promoter fragment. The amplified fragment was subcloned into the TA-Cloning vector (Invitrogen). The base sequence of each insert was determined using the DSQ 1000 fully automated DNA sequencer (Shimadzu Corporation). As a result, it was found that the SREBP-1c promoter of C3H/He Slc and CBA/JN Crj mice had the base sequence shown by SEQ ID NO:6, and that in DBA/2N Crj, DBA/1JN Crj and C57BL/6N Jcl mice, the guanine shown by base number 749 (base number 112 in the base sequence shown by SEQ ID NO:1) (G₁₁₂) has been substituted by adenine in the base sequence shown by SEQ ID NO:6. The relationship between metabolic responses of mice to feeding and the base substitution was examined; in C3H/He Slc and CBA/JN Crj mice, which have a SREBP-1c promoter comprising G₁₁₂, the serum TG concentration rose remarkably after meals (FR group) compared to during fasting (FF group), whereas in DBA/2N Crj, DBA/1JN Crj and C57BL/6N Jcl mice, which have an SREBP-1c promoter with G₁₁₂ substituted by adenine, no significant difference in blood TG concentration was observed between during fasting (FF group) and after meals (FR group) (Table 3).

TABLE 3

| | | Serum TG concentration (mg/dl) | |
|---|---|---|---|
| | 112-position base | | After meals |
| Mouse strain | (SEQ ID NO: 1) | During fasting (FF) | (FR) |
| C3H/He Slc | G | 80.3 ± 22.5 | 181 ± 19.9* |
| CBA/JN Crj | G | 142 ± 22.7 | 419 ± 17.8* |
| DBA/2N Crj | A | 108 ± 63.2 | 126 ± 56.2 |
| DBA/1JN Crj | A | 67.5 ± 20.6 | 92.3 ± 26.3 |
| C57BL/6N Jcl | A | 54 ± 13.9 | 56.5 ± 10.9 |

*p < 0.001 vs FF

### EXAMPLE 3

Differences in Lipid Metabolism Associated Gene Expression Responses to Feeding among Various Mouse Strains

After DBA/2N Crj and CBA/JN Crj mice (5-weeks old, male; purchased from Clea Japan, Inc.) were raised using the same feeding method as Example 1 (4-6 animals per group), they were anesthetized at 10:00 a.m. and the livers were resected, immediately frozen in liquid nitrogen and stored at −80° C. Also, serum triglyceride (TG) concentrations were measured according to a conventional method.

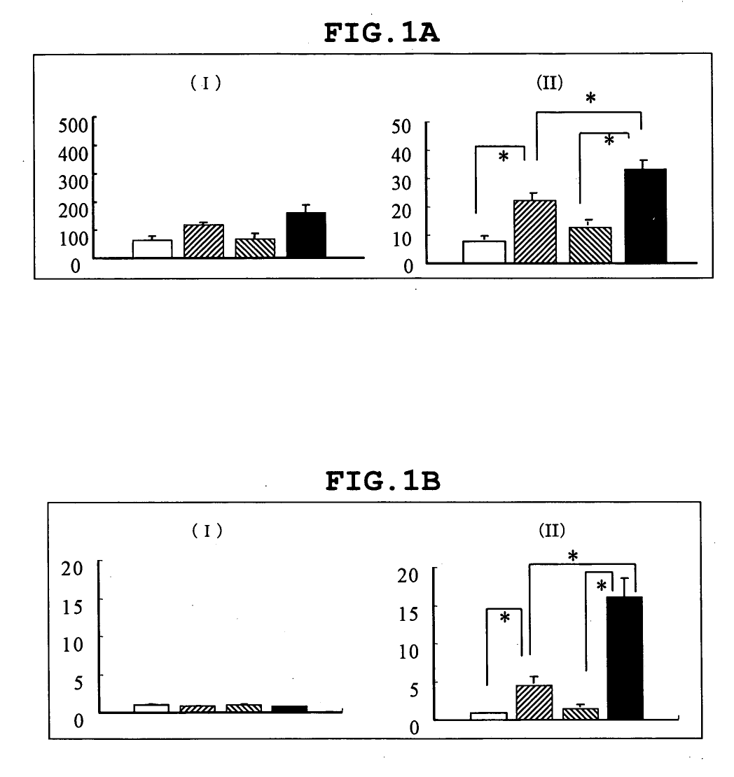

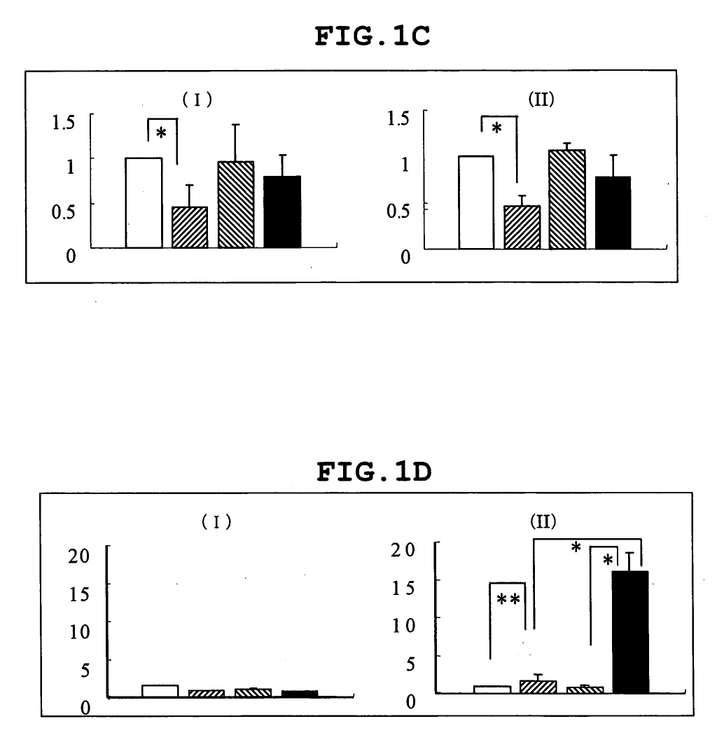

Total RNA was isolated from each frozen liver using a TRIzol reagent (GIBCO-BRL Life Technologies, Inc.), The RNA was electrophoresed in a formamide-containing 1% agarose gel and transferred onto a Hybond-N membrane (Amersham Pharmacia Biotech) . Probes for SREBP-1, PPAR-α and fatty acid synthase (FAS) mRNA were prepared according to the method described in Am J Physiol Endocrinol Metab 282: E1180-E1190, 2002. The probes were labeled with [α-³²P]dCTP (New England Nuclear Research Products) using a labeling kit (Takara). The membrane was hybridized to each radiolabeled probe in the Perfecthyb Buffer (Toyobo), and washed in 1×SSC, 0.1% SDS at 68° C. for 1 hour. The blot was exposed to a Kodak Biomax MR (Eastman Kodak) film at −80° C. The resulting signals were quantified using a densitometer, and loading differences were standardized against a signal obtained using a probe for 18S ribosome RNA. The results are shown in FIG. 1.

In DBA/2 Crj mice, no significant difference in serum TG concentration was observed among the four groups (CF, CR, FF, FR); whereas in CBA/JN Crj mice, the serum TG concentration rose significantly after meals (CR, FR) compared to during fasting (CF, FF) for both the ordinary diet and the high-fructose diet, with greater rises in TG concentration produced by the high-fructose diet (FR) than by the ordinary diet (CR). In starvation, no significant difference was observed between the ordinary diet (CF) and the high-fructose diet (FF) (FIG. 1A). The expression amount of SREBP-1c mRNA correlated well with the serum TG concentration (FIG. 1B). FAS, which is known to undergo expression control by SREBP-1c, exhibited the same expression pattern as SREBP-1c (FIG. 1D). On the other hand, there was no difference in the expression of PPARα mRNA to control the expression of fat decomposing enzymes between DBA/2 Crj and CBA/JN Crj, with decreased expression observed after meals compared during fasting in both strains of mice (FIG. 1C).

### EXAMPLE 4

Differences in Lipid Metabolism Associated Gene Expression Responses of Primary Hepatocytes to Sugar Stimulation among Various Mouse Strains

Liver cells were isolated from mice (DBA/2 Crj and CBA/JN Crj (purchased from Clea Japan, Inc.)) in the CF group and the FR group raised in the same manner as Example 1, using the collagenase method with a partial modification. The animals were anesthetized and each liver was perfused with Krebs-Ringer buffer solution (KRB) through the portal vein in situ. Subsequently, the liver was perfused with 100 ml of KRB containing collagenase (Sigma-Aldrich). After the dissociated cells were dispersed with shaking, the resulting dispersion was filtered through a gauge at 4° C. in an equal volume of ice cooled DMEM (GIBCO-BRL Life Technology) containing 10% (v/v) fetal calf serum (FCS), 100 μg/ml streptomycin and 100 U/ml penicillin.

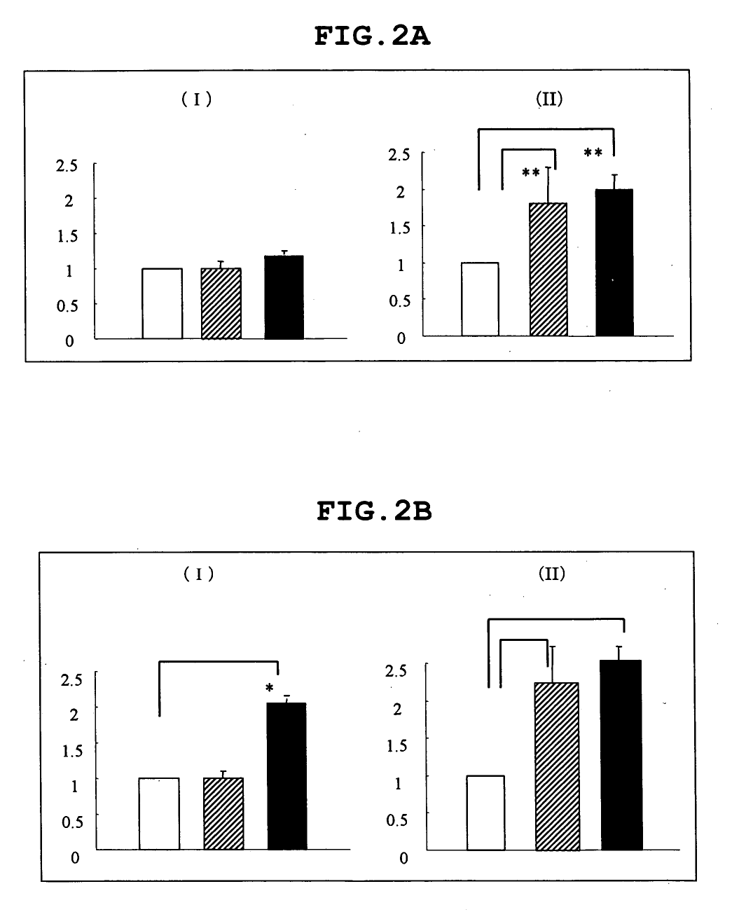

The cells were precipitated and twice washed with the same medium at 4° C. An aliquot of 8×10⁶ cells in William's E medium (Sigma-Aldrich) supplemented with 10% (v/v) FCS, 1 nM insulin, 100 nM triiodothyronine, 100 nM dexamethasone, 100 U/ml penicillin and 100 μg/ml streptomycin was seeded onto a 6-well plate coated with rat collagen. After incubation in 9% CO₂ at 37° C. for 3 hours, the cells were twice washed with PBS, and incubated using William's E-medium supplemented with 1 nM insulin, 100 nM triiodothyronine, 100 nM dexamethasone, 100 U/ml penicillin and 100 μg/ml streptomycin. After incubation for 16 hours, the cells were transferred to William's E medium supplemented-with 5 mM glucose, 5 mM fructose or 5 mM glucose +100 nM insulin. After incubation for a given time, the cells were recovered, and extraction of total RNA and Northern hybridization using the SREBP-1c or FAS cDNA probe were performed in the same manner as Example 3. The results are shown in FIG. 2.

An in vitro experiment using primary hepatocytes * revealed that in CAB/JN Crj mice, stimulation with fructose alone enhanced the expression of SREBP-1c (FIG. 2A) and FAS (FIG. 2B) mRNA to the same level as with insulin stimulation, but in DBA/2 JN Crj mice, no significant difference in the expression of SREBP-1c mRNA was observed among the different stimulations; no difference was observed in the expression of FAS mRNA as well between glucose stimulation and fructose stimulation. Because the expression of FAS mRNA increases with insulin stimulation also in DBA/2 JN Crj mice, it is suggested that a regulatory factor other than SREBP-1c may be involved in the control of FAS expression responses to insulin.

### EXAMPLE 5

Identification of a Transcriptional Regulatory Factor that Binds to a Fructose Responsive Element in the SREBP-1c Promoter

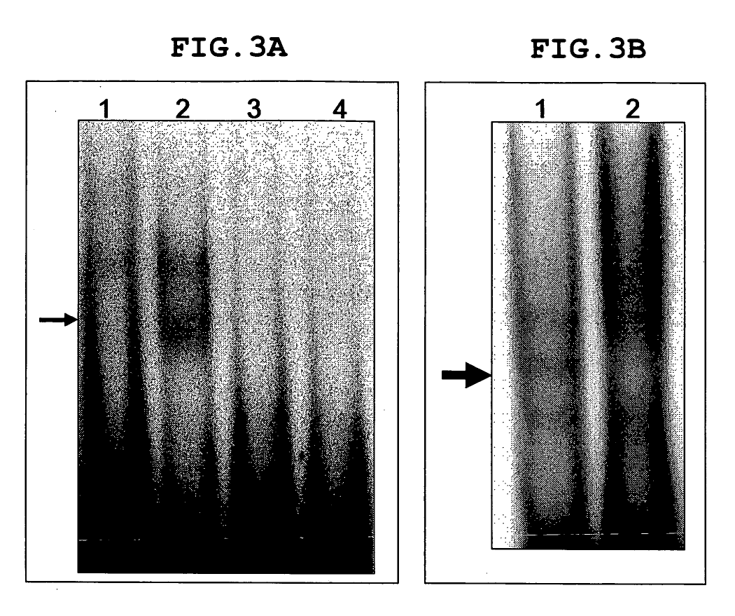

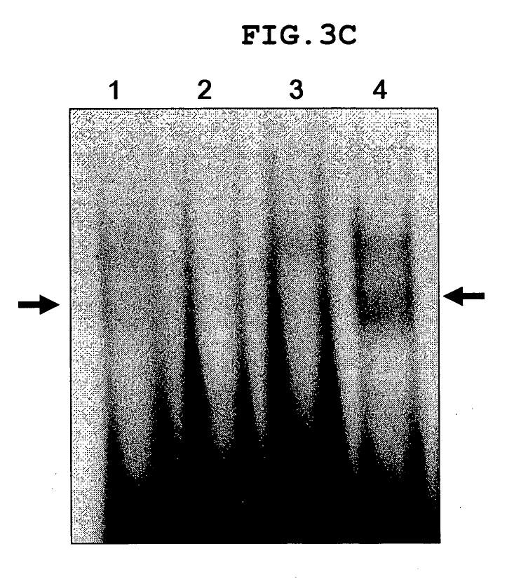

The livers were resected from mice (DBA/2 Crj and CBA/JN Crj (purchased from Clea Japan, Inc.)) in the CF group and the FR group raised in the same manner as Example 1, and a nuclear protein extract from hepatocytes was isolated in accordance with the method of Gorski et al. (Cell 47: 767-776, 1986). The nuclear extract was suspended in 20 mM HEPES (pH 7.9), 330 mM NaCl, 1.5 mM MgCl₂, 0.2 mM EDTA, 25% glycerol, 0.5 mM dithiothreitol and 0.2 mM PMSF, and an aliquot of the resulting suspension was frozen in liquid nitrogen and stored at −80° C. Separately, two kinds of radiolabeled double-stranded oligonucleotides comprising the base sequence of G₁₁₂ in the SREBP-1c promoter of CBA/JN Crj mice and in the vicinity thereof (5′-CTAAAGGCAGCTATTGGCCT-3′; SEQ ID NO:9) and the base sequence of the corresponding region in the SREBP-1c promoter of DBA/2 JN Crj mice (5′-CTAAAGGCAACTATTGGCCT-3′; SEQ ID NO:10), respectively (called CBA probe and DBA probe, respectively) were synthesized. An electrophoretic migration shift assay was conducted using these oligonucleotides. 10 μg of nuclear extract, 1 μg of poly (dI-dC), 10 mM HEPES (pH 7.9), 60 mM KCL, 1 mM EDTA, 7% glycerol, and 100,000 cpm labeled probe were mixed and incubated at room temperature for 20 minutes to cause a protein-DNA binding reaction. After incubation, the sample was loaded to 6% polyacrylamide gel in 0.25×Tris-borate-EDTA (TBE) buffer, and electrophoresed at a voltage of 150 V. After electrophoresis, the gel was dried and exposed to a film. A competitive assay using a non-labeled oligonucleotide (cold probe) was also performed, and the band that disappeared in the presence of the cold probe was identified as a band of the probe-specifically bound transcriptional regulatory factor. The results are shown in FIG. 3.

The band observed when CBA/JN Crj mouse-derived nuclear extract in the FR group was reacted with the CBA probe (FIG. 3A; arrow) was not observed when the same extract was reacted with the DBA probe. Thus, the presence of a transcriptional regulatory factor that specifically binds to G₁₁₂ in the CBA/JN Crj mouse SREBP-1c promoter and a base sequence in the vicinity thereof was confirmed.

When the DBA/2 JN Crj mouse-derived nuclear extract was reacted with the CBA probe, the band observed when the CBA/JN Crj mouse-derived nuclear extract was reacted with the CBA probe (FIG. 3B; arrow) showed weak signal intensity; it is suggested that the reason why the expression of SREBP-1c does not increase in response to high-fructose diet loading because not only a promoter mutation but also an expression insufficiency of the binding protein may have an effect in DBA/2 JN Crj mice.

Furthermore, when the CBA/JN Crj mouse-derived nuclear extract from the CF group was reacted with the CBA probe, the band observed when the CBA/JN Crj mouse-derived nuclear extract in the FR group is, reacted with the CBA probe (FIG. 3C; arrow) exhibited very weak signal, showing a good correlation with the expression of SREBP-1c mRNA in each group. Thus, it was suggested that the SREBP-1c expression response to feed in CBA/JN Crj mice might be controlled by the expression of a transcriptional regulatory factor that binds to the fructose responsive element in the SREBP-1c promoter.

### EXAMPLE 6

Amino Acid Sequencing of a Transcriptional Regulatory Factor that Binds to the Fructose Responsive Element in the SREBP-1c Promoter

Livers were excised from CBA/JN Crj mice during fasting and at 2 hours after ingestion of a fructose diet, and nuclear protein was extracted in the same manner as Example 5. Two synthetic DNAs (5′-AATTCTAAAGGCAGCTATTGGCCT-3′: SEQ ID NO:11; 5′-AATTGGCCAATAGCTGCCTTTAG-3′: SEQ ID NO:12) were hybridized to yield a double-stranded DNA comprising G₁₁₂ in the SREBP-1c promoter of CBA/JN Crj mice and a base sequence in the vicinity thereof, and linked to EasyAnchor EcoRI-N (Nippon Gene Co., Ltd., Tokyo) using a TaKaRa ligation kit. 50 μl of EasyAnchor and 200 μg of nuclear protein extract were allowed to stand in the binding buffer used in gel shift analysis at room temperature for 30 minutes. After centrifugation (15,000 rpm, 1 min, 4° C.), the precipitate was washed with a washing buffer (100 mM KCl, 15 mM HEPES-KOH (pH 7.9), 25 mM EDTA, 1 mM DTT, 0.1 mM PMSF, 10%. (w/v) glycerol) at 4° C. five times, and thereafter the protein was eluted at room temperature with 100 μl of an elution buffer (1.5M KCl, 15 mM HEPES-KOH (pH 7.9), 25 mM EDTA, 1 mM DTT:, 0.1 mM PMSF, 10% (w/v) glycerol). After centrifugation (15,000 rpm, 1 min, 4° C.), the salts in the supernatant were removed by a conventional method, and an electrophoregram was developed by SDS-PAGE. After electrophresis, the gel was stained with silver staining to visualize protein bands; two bands near 41 to 45 kDa that showed a major difference between the sample taken during fasting and the sample taken after ingestion of the fructose diet were cleaved from the gel, and subjected to MALDI-TOF-MS analysis (outsourced to Shimadzu Corporation at Tsukuba) to analyze the primary structures of the proteins. As a result, these proteins proved to be identical to the publicly known Nonamer Binding Protein (GenBank accession number: AAA81558 (SEQ ID NO:3); cDNA was M88489. (SEQ ID NO:2), and to a protein similar to the RNA binding motif protein, X chromosome retrogene (GenBank accession number: AAH11441 (SEQ ID NO:5); cDNA was BC011441 (SEQ ID NO:4).

### EXAMPLE 7

Search for a Human SREBP-1c Promoter Sequence Homologous to the Fructose Responsive Element in the Mouse SREBP-1c Promoter

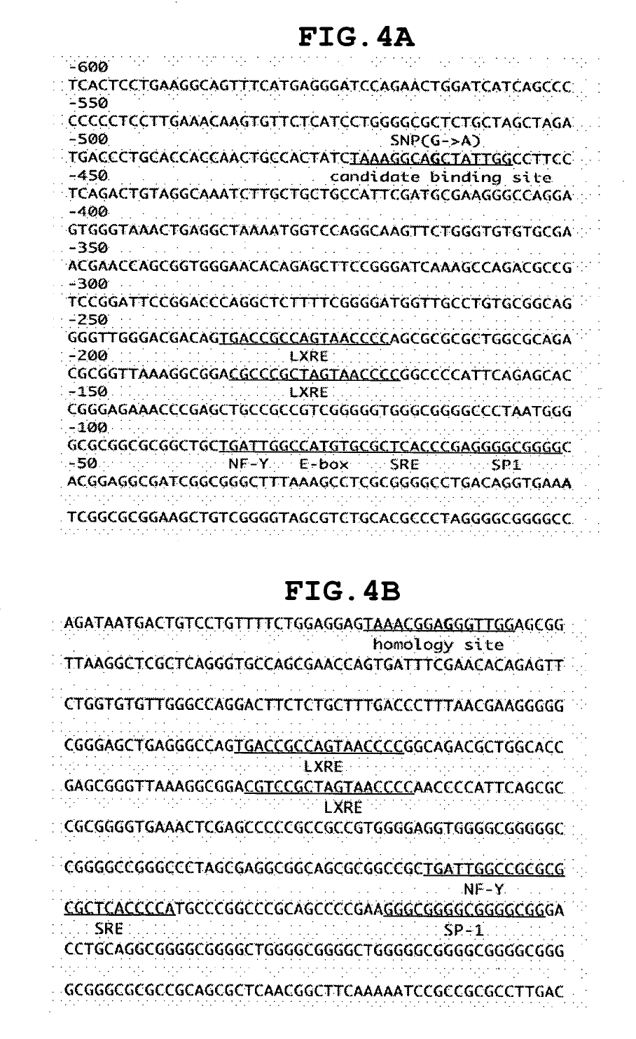

The SREBP-1c promoter sequence of CBA/JN Crj mice and a complementary chain sequence of the base sequence shown by base numbers 16566061-16566560 (SEQ ID NO:13) in the base sequences of human chromosome number 17 (accession number: NT_{—}010718) registered in GenBank, corresponding to the promoter region of the human SREBP-1c gene, were compared using a DNASIS-homology search program. The results are shown in FIG. 4. A sequence homologous to the mouse fructose responsive element (“homology site” in FIG. 4B) was also found in the human promoter.

### INDUSTRIAL APPLICABILITY

The fructose responsive element of the present invention, a transcriptional regulatory factor that interacts therewith, and a non-human animal having them transferred or inactivated are not only useful in elucidating the mechanism of induction of a metabolic disorder, but also useful in diagnosing genetic susceptibility to a metabolic disorder, screening for a prophylactic or therapeutic agent for a metabolic disorder and the like.

1. A nucleic acid consisting of the same or substantially the same base sequence as a portion of the base sequence shown by SEQ ID NO:1, and comprising the guanine shown by base number 112 in the base sequence.

2. A nucleic acid characterized by (1) and (2) below: (1) comprises a base sequence having one or more bases substituted, deleted, inserted or added in the same or substantially the same base sequence as a portion of the base sequence shown by SEQ ID NO:1, and comprising the base sequence or the guanine shown by base number 112 (2) a transcriptional regulatory factor capable of binding to a base sequence consisting of the guanine shown by base number 112 and a base adjoining thereto in the base sequence shown by SEQ ID NO:1 cannot bind to the nucleic acid.

3. The nucleic acid of claim 2, wherein the guanine shown by base number 112 in the base sequence shown by SEQ ID NO:1 is substituted by another base.

4. The nucleic acid of claim 3, wherein the another base is adenine.

5. A diagnostic method for genetic susceptibility to a metabolic disorder in a test animal, which comprises detecting a portion of the base sequence shown by SEQ ID NO:1 comprising the guanine shown by base number 112 in the base sequence, or a corresponding base sequence, in the SREBP-1c promoter.

6. The method of claim 5, wherein the metabolic disorder is a sugar or lipid metabolic disorder.

7. A screening method for a prophylactic or therapeutic substance for a metabolic disorder, which comprises using both (a) below and (b) and/or (c) below,
(a) a DNA having the same or substantially the same base sequence as a portion of the base sequence shown by SEQ ID NO:1, comprising the base sequence or the guanine shown by base number 112,
(b) a protein comprising the same or substantially the same amino acid sequence as the amino acid sequence shown by SEQ ID NO:3 or a partial peptide thereof or a salt thereof
(c) a protein comprising the same or substantially the same amino acid sequence as the amino acid sequence shown by SEQ ID NO:5 or a partial peptide thereof or a salt thereof.

8. The method of claim 7, wherein the metabolic disorder is a sugar or lipid metabolic disorder.

9. The method of claim 7, which comprises detecting the inhibition of the binding of said (a) and said (b) and/or (c) in the presence of a test substance.

10. The method of claim 7, which comprises loading a sugar on an animal cell having a gene under the control of a promoter comprising said (a), and comparing the expression of the gene between in the presence and in the absence of a test substance.

11. The method of claim 10, wherein the animal cell is capable of producing said (b) and/or (c).

12. The method of claim 10, wherein the animal cell is a hepatocyte.

13. The method of claim 10, wherein the sugar is fructose.

14. The method of claim 10, which comprises loading a sugar on an animal having a gene under the control of a promoter comprising a DNA having the same or substantially the same base sequence as a portion of the base sequence shown by SEQ ID NO:1, and comprising the guanine shown by base number 112 in the base sequence, and comparing the expression of the gene in the liver between with and without administration of a test substance.

15. The method of claim 14, wherein the sugar is fructose.

16. A prophylactic or therapeutic method for a metabolic disorder, which comprises administering an effective amount of a substance that suppresses the production or activity of (a) and/or (b) below to a mammal,
(a) a protein having the same or substantially the same amino acid sequence as the amino acid sequence shown by SEQ ID NO:3 or a partial peptide thereof or a salt thereof;
(b) a protein having the same or substantially the same amino acid sequence as the amino acid sequence shown by SEQ ID NO:5 or a partial peptide thereof or a salt thereof.

17. The method of claim 16, wherein the metabolic disorder is a sugar or lipid metabolic disorder.

18. The method of claim 16, wherein the substance that suppresses the activity is an antibody against said (a) and/or an antibody against said (b).

19. The method of claim 16, wherein the substance that suppresses the activity is a DNA having the same or substantially the same base sequence as a portion of the base sequence shown by SEQ ID NO:1, and comprising the guanine shown by base number 112 in the base sequence.

20. The method of claim 16, wherein the substance that suppresses the production is (c) and/or (d) below,
(c) a nucleic acid comprising a base sequence complementary to the base sequence that encodes said (a), or a portion thereof
(d) a nucleic acid comprising a base sequence complementary to the base sequence that encodes said (b), or a portion thereof.

21. (canceled)

22. (canceled)

23. A protein or peptide characterized by (1) and (2) below or a salt thereof:
(1) comprises an amino acid sequence having one or more amino acids substituted, deleted, inserted, or added, in a protein comprising the same or substantially the same amino acid sequence as the amino acid sequence shown by SEQ ID NO:3 or a partial peptide thereof or a salt thereof,
(2) binds to the same or substantially the same base sequence as a portion of the base sequence shown by SEQ ID NO:1, comprising the guanine shown by base number 112 in the base sequence, but does not activate promoters comprising the base sequence.

24. A protein or peptide characterized by (1) and (2) below or a salt thereof:
(1) comprises an amino acid sequence having one or more amino acids substituted, deleted, inserted, or added, in a protein comprising the same or substantially the same amino acid sequence as the amino acid sequence shown by SEQ ID NO:5 or a partial peptide thereof or a salt thereof,
(2) binds to the same or substantially the same base sequence as a portion of the base sequence shown by SEQ ID NO:1, comprising the guanine shown by base number 112 in the base sequence, but does not activate promoters comprising the base sequence.

25. A prophylactic or therapeutic method for a metabolic disorder, which comprises administering an effective amount of the protein or peptide of claim 23 or a salt thereof, and/or the protein or peptide of claim 24 or a salt thereof to a mammal.

26. A diagnostic method for a metabolic disorder, which comprises using (a) and/or (b) below:
(a) an antibody against a protein comprising the same or substantially the same amino acid sequence as the amino acid sequence shown by SEQ ID NO:3 or a partial peptide thereof or a salt thereof
(b) an antibody against a protein comprising the same or substantially the same amino acid sequence as the amino acid sequence shown by SEQ ID NO:5 or a partial peptide thereof or a salt thereof.

27. A diagnostic method for a metabolic disorder, which comprises using (a) and/or (b) below:
(a) a nucleic acid comprising the base sequence that encodes a protein having the same or substantially the same amino acid sequence as the amino acid sequence shown by SEQ ID NO:3 or a portion thereof
(b) a nucleic acid comprising the base sequence that encodes a protein having the same or substantially the same amino acid sequence as the amino acid sequence shown by SEQ ID NO:5 or a portion thereof.

28. A non-human transgenic animal incorporating a gene under the control of a promoter comprising a DNA having the same or substantially the same base sequence as a portion of the base sequence shown by SEQ ID NO:1, and comprising the guanine shown by base number 112 in the base sequence.

29. The non-human transgenic animal of claim 28, wherein an endogenous SREBP-1c gene characterized by (1) below:
(1) comprises a promoter to which (a) and/or (b) below cannot bind:
(a) a protein comprising the same or substantially the same amino acid sequence as the amino acid sequence shown by SEQ ID NO:3 or a partial peptide thereof or a salt thereof
(b) a protein comprising the same or substantially the same amino acid sequence as the amino acid sequence shown by SEQ ID NO:5 or a partial peptide thereof or a salt thereof is substituted by an SREBP-1c gene characterized by (2) below:
(2) is under the control of a promoter comprising a DNA having the same or substantially the same base sequence as a portion of the base sequence shown by SEQ ID NO:1, and comprising the guanine shown by base number 112 in the base sequence.

30. A non-human transgenic animal incorporating a gene under the control of a promoter characterized by (1) and (2) below:
(1) comprises a DNA having one or more bases substituted, deleted, inserted, or added, in the same or substantially the same base sequence as a portion of the base sequence shown by SEQ ID NO:1, and comprising the guanine shown by base number 112 in the base sequence,
(2) (a) and/or (b) below cannot bind:
(a) a protein comprising the same or substantially the same amino acid sequence as the amino acid sequence shown by SEQ ID NO:3 or a partial peptide thereof or a salt thereof
(b) a protein comprising the same or substantially the same amino acid sequence as the amino acid sequence shown by SEQ ID NO:5 or a partial peptide thereof or a salt thereof.

31. The non-human transgenic animal of claim 30, wherein the endogenous SREBP-1c gene comprising a promoter having the same or substantially the same base sequence as the base sequence shown by SEQ ID NO:1 is substituted by an SREBP-1c gene under the control of a promoter characterized by said (1) and (2).

32. A non-human transgenic animal incorporating (a) and/or (b) below:
(a) a DNA that encodes a protein comprising the same or substantially the same amino acid sequence as the amino acid sequence shown by SEQ ID NO:3 or a partial peptide thereof
(b) a DNA that encodes a protein comprising the same or substantially the same amino acid sequence as the amino acid sequence shown by SEQ ID NO:5 or a partial peptide thereof.

33. A non-human animal having (a) and/or (b) below inactivated:
(a) a DNA that encodes a protein comprising the same or substantially the same amino acid sequence as the amino acid sequence shown by SEQ ID NO:3
(b) a DNA that encodes a protein comprising the same or substantially the same amino acid sequence as the amino acid sequence shown by SEQ ID NO:5.


SEQUENCE LISTING (13 sequences)

SEQ ID NO: 1 - DNA, 574 bp, CBA mouse
  promoter (1)..(574)
  TATA_signal (545)..(550)
ggatccagaactggatcatcagcccccccctccttgaaacaagtgttctcatcctggggcgctctgctagctagatgaccctgcaccaccaactgccactatctaaaggcagctattggccttcctcagactgtaggcaaatcttgctgctgccattcgatgcgaagggccaggagtgggtaaactgaggctaaaatggtccaggcaagttctgggtgtgtgcgaacgaaccagcggtgggaacacagagcttccgggatcaaagccagacgccgtccggattccggacccaggctcttttcggggatggttgcctgtgcggcaggggttgggacgacagtgaccgccagtaaccccagcgcgcgctggcgcagacgcggttaaaggcggacgcccgctagtaaccccggccccattcagagcaccgggagaaacccgagctgccgccgtcgggggtgggcggggccctaatggggcgcggcgcggctgctgattggccatgtgcgctcacccgaggggcggggcacggaggcgatcggcgggctttaaagcctcgcggggcctgacaggtgaa

SEQ ID NO: 2 - DNA, 1284 bp, Mus musculus
  CDS (1)..(1284)
atggtggagaacaggatcatctatgactcagactcagagtcagaggagacagtacaggtaaaaaatgctaaaaagaaatcagaaaaattgtcactgtcttataaacctggtaaagtctctcagaaggatcctgtcacctacgtctctgagacagatgaagatgatgactttgtatgtaaaaaggcagcctccaaatcaaaagagaatggagtatctacaaatagttaccttggaacatcaaacgtgaaaaaaaatgaagaaaacgttaagactaagaacaagccgttatctccaataaaactcacaccaacgtcagtgctcgactattttggaactgaaagtgtccagagatctgggaagaagatggtgacaagcaaaaggaaagaatcttctcaaaacacagaggattccagattaaatgatgaggccatcgccaagcagctgcagctggatgaagatgcagagctggagaggcagttgcatgaagatgaagaatttgcaagaacactggccttattggatgaagaacccaagatcaaaaaggcccgaaaggattctgaagagggagaagaatcattttcatctgtccaagatgatttaagcaaagcagaaaagcagaaaagccctaataaagccgagcttttctcaactgcaagaaagacctacagtcctgctaagcatggcaaggggagggcttcagaagatgctaagcagccttgcaaatcagctcaccggaaggaagcctgttcctcacccaaggccagtgccaaactggcgcttatgaaagcaaaagaagaaagttcttacaacgaaacagagctgctggctgcaagaagaaaagaaagcgccactgaacccaaaggagagaaaacaactcctaagaaaacgaaagtttctccaactaagagagagtctgtaagcccagaagactctgaaaagaagcgcactaattatcaagcttatcgaagctacttgaaccgagaaggtcccaaagctctgggctccaaagaaatacccaagggagctgaaaattgcttggaaggcctgacgtttgtgatcacaggagtgctggagtccattgaacgagacgaagccaagtctctaattgaacgttatggggggaaagtaacaggaaacgtgagcaagaaaaccaactacctcgtcatgggccgcgacagtgggcagtccaagagtgacaaggcagcagctctgggaacaaaaatcctcgatgaagacggcctgttggatctgattcgaactatgccaggcaagagatccaagtac

SEQ ID NO: 3 - protein, 428 aa, Mus musculus
MVENRIIYDSDSESEETVQVKNAKKKSEKLSLSYKPGKVSQKDPVTYVSETDEDDDFVCKKAASKSKENGVSTNSYLGTSNVKKNEENVKTKNKPLSPIKLTPTSVLDYFGTESVQRSGKKMVTSKRKESSQNTEDSRLNDEAIAKQLQLDEDAELERQLHEDEEFARTLALLDEEPKIKKARKDSEEGEESFSSVQDDLSKAEKQKSPNKAELFSTARKTYSPAKHGKGRASEDAKQPCKSAHRKEACSSPKASAKLALMKAKEESSYNETELLAARRKESATEPKGEKTTPKKTKVSPTKRESVSPEDSEKKRTNYQAYRSYLNREGPKALGSKEIPKGAENCLEGLTFVITGVLESIERDEAKSLIERYGGKVTGNVSKKTNYLVMGRDSGQSKSDKAAALGTKILDEDGLLDLIRTMPGKRSKY

SEQ ID NO: 4 - DNA, 1164 bp, Mus musculus
  CDS (1)..(1164)
atggttgaagcagatcgcccaggaaagctcttcattggtgggcttaatacagaaacaaatgagaaagcccttgaagcagtatttggcaagtatggacgaatagtggaaatacttttgatgaaagaccgggaaaccaacaaatcaagaggatttgcttttgttaccttcgaaagcccagcagatgctaaagatgcagctagagatatgaatggaaagtccttggatggaaaagccatcaaggtggagcaagctaccaaaccatcttttgaaagtggcaggcgtggaccacctccacctccaagaagcaggggccctcccagaggtcttcgaggaggaagtggaggaactaggggacccccttcacgtggaggatacatggatgatggtggttattccatgaactttaacatgagttcttccaggggaccacttccagtaaagagaggaccaccaccacgaagcgggggtccccctcctaaaagatcaacaccttccggaccagttcgaagcagcagtggaatgggcggaagaacaccagtgtcccgtggaagagatagctacgggggtccaccacgaagggaacccctgccatctcgcagagatgtttatttgtccccaagagatgatggatattctacaaaagatagctattcaagcagagattacccaagctcccgagacaccagagattatgcaccgccaccaagagattatacttaccgtgattacagtcattccagttcccgtgatgactatccatcaagaggctacggtgatagagatggatatggtcgggatcgtgactattcagatcatccaagtggaggttcctacagagattcatatgagagttatggaaactcacgcagtgcaccccctacacgagggccaccaccatcttatggaggaagcagtcgctatgatgattacagcagctcacgcgatggctacggtggaagtcgagacagttactcaagcagcagaagtgatctctactcaagtggccgtgatcgagttggcagacaagaacgagggcttcccccttctatggaaagggggtaccctcctccacgtgattcctacagcagttcaagccgaggagcgccaagaggaggtggccgtggagggagccgatctgatagagggggaggcagaagcagatat

SEQ ID NO: 5 - protein, 388 aa, Mus musculus
MVEADRPGKLFIGGLNTETNEKALEAVFGKYGRIVEILLMKDRETNKSRGFAFVTFESPADAKDAARDMNGKSLDGKAIKVEQATKPSFESGRRGPPPPPRSRGPPRGLRGGSGGTRGPPSRGGYMDDGGYSMNFNMSSSRGPLPVKRGPPPRSGGPPPKRSTPSGPVRSSSGMGGRTPVSRGRDSYGGPPRREPLPSRRDVYLSPRDDGYSTKDSYSSRDYPSSRDTRDYAPPPRDYTYRDYSHSSSRDDYPSRGYGDRDGYGRDRDYSDHPSGGSYRDSYESYGNSRSAPPTRGPPPSYGGSSRYDDYSSSRDGYGGSRDSYSSSRSDLYSSGRDRVGRQERGLPPSMERGYPPPRDSYSSSSRGAPRGGGRGGSRSDRGGGRSRY

SEQ ID NO: 6 - DNA, 1187 bp, CBA mouse
  promoter (1)..(1187)
  TATA_signal (1182)..(1187)
agaaaggggatcagggcaggctggacagaacggtgtcataaaaagatgttttctttggaatgaacctcctatgaggatgtgaaaagacctagaaaggggatcaggggaatgtcagacacacgtgtctgtttcccagacaagactctgaaaagagagatgggccacaagtccctgacacacataaggtgactacttggtcgctggacccctcacagactgtgtgagtccctggtctgccaactaggctgccagaccttgctgggccactgccacagaagctaggttgctggccatcactgtgtggtgatggtaatggcgggagtatgtgtgtgcacatgcttgtgtgtgcacaggtatgaaagctttcaatttgccagcaagggacagggacagatttggcatacctttaatatccactgcctttcccttctgtcccagagactggttcctgtgcaggcctttgcagagtgctataagagaatcgagtaaggcttcacttgttgactgctgggggctgtgatacctggagggaagacactgacccagcctaggggcatcagagctgagagcaggatatcctggacgcgtgatttgaggaaggatttccctagctcactcctgaaggcagtttcatgagggatccagaactggatcatcagcccccccctccttgaaacaagtgttctcatcctggggcgctctgctagctagatgaccctgcaccaccaactgccactatctaaaggcagctattggccttcctcagactgtaggcaaatcttgctgctgccattcgatgcgaagggccaggagtgggtaaactgaggctaaaatggtccaggcaagttctgggtgtgtgcgaacgaaccagcggtgggaacacagagcttccgggatcaaagccagacgccgtccggattccggacccaggctcttttcggggatggttgcctgtgcggcaggggttgggacgacagtgaccgccagtaaccccagcgcgcgctggcgcagacgcggttaaaggcggacgcccgctagtaaccccggccccattcagagcaccgggagaaacccgagctgccgccgtcgggggtgggcggggccctaatggggcgcggcgcggctgctgattggccatgtgcgctcacccgaggggcggggcacggaggcgatcggcgggctttaaa

SEQ ID NO: 7 - DNA, 20 bp, Artificial Sequence
  Oligonucleotide designed to act as primer for amplifying promoter region (ca. 1.2 kb upstream from TATA-like sequence) of SREBP-1c gene.
gctggacagaacggtgtcat

SEQ ID NO: 8 - DNA, 26 bp, Artificial Sequence
  Oligonucleotide designed to act as primer for amplifying promoter region (ca. 1.2 kb upstream from TATA-like sequence) of SREBP-1c gene.
  misc_feature: Oligonucleotide designed to act as primer for amplifying promoter region (ca. 1.2 kb upstream from TATA-like sequence) of SREBP-1c gene.
taagagctcggtacctcccctagggc

SEQ ID NO: 9 - DNA, 20 bp, Artificial Sequence
  Oligonucleotide corresponding to fructose responsive element in SREBP-1c promoter of CBA/JN Crj mouse.
ctaaaggcagctattggcct

SEQ ID NO: 10 - DNA, 20 bp, Artificial Sequence
  Oligonucleotide corresponding to mutated fructose responsive element in SREBP-1c promoter of DBA/2 JN Crj mouse.
ctaaaggcaactattggcct

SEQ ID NO: 11 - DNA, 24 bp, Artificial Sequence
  Oligonucleotide corresponding to sense strand of fructose responsive element in SREBP-1c promoter of CBA/JN Crj mouse.
aattctaaaggcagctattggcct

SEQ ID NO: 12 - DNA, 23 bp, Artificial Sequence
  Oligonucleotide corresponding to antisense strand of fructose responsive element in SREBP-1c promoter of CBA/JN Crj mouse.
aattggccaatagctgcctttag

SEQ ID NO: 13 - DNA, 500 bp, Homo sapiens
  promoter (1)..(500)
agataatgactgtcctgttttctggaggagtaaacggagggttggagcggttaaggctcgctcagggtgccagcgaaccagtgatttcgaacacagagttctggtgtgttgggccaggacttctctgctttgaccctttaacgaagggggcgggagctgagggccagtgaccgccagtaaccccggcagacgctggcaccgagcgggttaaaggcggacgtccgctagtaaccccaaccccattcagcgccgcggggtgaaactcgagcccccgccgccgtggggaggtggggcgggggccggggccgggccctagcgaggcggcagcgcggccgctgattggccgcgcgcgctcaccccatgcccggcccgcagccccgaagggcggggcggggcgggacctgcaggcggggcggggctggggcggggctgggggcggggcggggcggggcgggcgcgccgcagcgctcaacggcttcaaaaatccgccgcgccttgac